(19) United States
(12) Patent Application Publication
(10) Pub. No.: US 2026/0049978 A1
(43) Pub. Date: Feb. 19, 2026

(54) METHODS FOR TREATING CAG REPEAT EXPANSION DISORDERS USING SMALL MOLECULES THAT SELECTIVELY REDUCE EXPANDED CAG TRANSCRIPT LEVELS

(71) Applicant: The Research Foundation for the State University of New York, Albany, NY (US)

(72) Inventors: Hannah K Shorrock, Albany, NY (US); John Douglas Berglund, Albany, NY (US); John Cleary, Albany, NY (US); Ashif Bhuiyan, Albany, NY (US)

(21) Appl. No.: 19/196,499

(22) Filed: May 1, 2025

Related U.S. Application Data
(60) Provisional application No. 63/640,924, filed on May 1, 2024.

(51) Int. Cl. G01N 33/50

(52) U.S. Cl. CPC G01N 33/5023; G01N 33/5044

(57) ABSTRACT

A cell-based screening system and method for identifying compounds that selectively modulate the expression of CAG repeat-containing RNA associated with spinocerebellar ataxias and related disorders. The system comprises a human HEK293T cell line engineered to co-express two reporter constructs: a CAG repeat-expanded polyglutamine-nanoluciferase fusion protein with at least 60 CAG repeats, and a control firefly luciferase with no CAG repeats. Each construct contains a unique probe-binding sequence downstream of the repeat region, enabling independent quantification via multiplex RT-qPCR with fluorescent probes, as well as dual luciferase assays. The cell line is optimized for high-throughput screening to identify therapeutic compounds that reduce pathogenic CAG repeat RNA levels while sparing control transcripts. The invention further encompasses methods for screening, validating, and identifying candidate therapeutics for CAG expansion disorders, including spinocerebellar ataxias and Huntington's disease.

### CROSS-REFERENCE TO RELATED APPLICATIONS

This application claims the benefit of the filing date of U.S. Provisional Application No. 63/640,924, filed May 1, 2024. The content of this earlier filed application is hereby incorporated by reference herein in its entirety.

### STATEMENT REGARDING FEDERALLY SPONSORED RESEARCH OR DEVELOPMENT

This invention was made with government support under grant number R01 NS135254 awarded by National Institutes of Health. The government has certain rights in the invention.

### BACKGROUND

Microsatellite repeat expansions cause over 50 neurodegenerative, neurological and neuromuscular diseases, including Huntington's disease (HD), Spinal and bulbar muscular atrophy (SBMA), C9orf72 amyotrophic lateral sclerosis/frontotemporal dementia (C9 ALSIFTD) and myotonic dystrophy types 1 and 2 (DM1, DM2). One of the most frequent class of disease-causing microsatellite expansions are CAG repeat expansions which are the genetic cause of HD, S8MA Dentatorubral pallidoluysian atrophy (DRPLA), and multiple subtypes of spinocerebellar ataxia (SCA) including types 1, 2, 3, 6, 7, 12 and 17. The SCAs are a genetically heterogenous group of rare neurodegenerative disorders that share the clinical feature of progressive ataxia. This loss of balance and coordination is accompanied by slurred speech, a loss of control of eye movements and reflects dysfunction and degeneration of the cerebellum and interconnected regions of the nervous system including the brainstem. For some SCAs, this dysfunction and degeneration is particularly focused on the Purkinje neurons in the cerebellum, however, for other SCAs, these are less affected.

While the precise molecular mechanism(s) responsible for disease pathogenesis remain unclear for CAG SCAs, they share the production of CAG expansion RNAs and the expression of toxic polyglutamine (polyQ) proteins. This is the case for SCAs 1, 2, 3, 6, 7 and 17 where the CAG repeat expansion mutation is located within the coding region of ATXN1, ATXN2, ATXN3, CACAN1A, ATXN7, and TBP respectively. The expanded polyglutamine tracts produced in these diseases alter protein conformation leading to disruption of normal function and protein aggregation. However, in the case of SCA12, the CAG repeat expansion is in the 5′UTR of PPP2R2B and polyQ proteins have not been reported in this disease.

As a consequence of the expression of these CAG expansion transcripts and subsequent polyQ proteins, various cellular processes have been implicated in CAG SCAs, including ion channel dysfunction, transcriptional dysregulation, and DNA damage; however, the precise molecular drivers of dysfunction for these pathways remains unclear. Across the SCAs, and indeed CAG expansion diseases, current therapeutic approaches focus primarily on treating symptoms, leaving a large unmet need for therapies that address underlying disease mechanisms in SCAs.

To target the underlying disease mechanism and the direct consequences of the CAG expansion mutations, several levels of the pathogenic cascade could be targeted: the CAG expansion containing gene, expansion transcripts, or expansion proteins. Alternatively, downstream consequences of this pathogenic cascade can also be targeted. While targeting each level of this pathogenic cascade comes with its own strengths and weaknesses, the further up this cascade that can be successfully targeted, the more likely it is that multiple downstream consequences of disease will be rescued.

While therapeutic strategies targeting each of these levels of the pathogenic cascade are currently in preclinical development for SCAs, one of the more advanced strategies, with clinical trials currently ongoing, is targeting disease specific mRNA transcripts by using antisense oligonucleotides. For example, ASOs (Antisense Oligonucleotides) targeting gene specific sequences reduce levels of the CAG expansion RNAs and the encoded polyglutamine expansion proteins as well as rescuing disease specific phenotypes in SCA1, 2, 3 and 7 mouse models. Indeed, a non-allele specific ASO currently in clinical trials for SCA3 (NCTOS160558) highlights the promise of this approach. Additionally, short hairpin RNAs and microRNAs that target degradation of expansion mRNAs have also shown therapeutic promise in preclinical studies, thereby demonstrating the effectiveness of targeting CAG expansion RNAs in these SCAs. The majority of these therapeutic approaches have been gene specific, offering hope for one subtype of SCA but not across the larger family of ataxias. Other ASO approaches targeting the CAG repeats directly highlight the potential of shared therapeutics across CAG SCAs. A CAG repeat-targeting ASO has been shown to reduce levels of CAG expansion RNAs and polyglutamine expansion proteins in mouse models of both SCA1 and SCA313 and is currently entering clinical trials for SCA1, 3 and HD (NCT05822908).

Despite this, the suspension of multiple clinical trials for ASOs in HD2, highlights the challenge of translating ASOs aimed at reducing levels of disease associated RNAs, from preclinical development to approved treatment. Therefore, it is critical to develop alternative therapeutic approaches, including those that could work independently or in combination with ASOs. Other therapeutic strategies aimed at targeting CAG expansion transcripts, or further upstream in the pathogenic cascade, in a cross-disease manner include RNA cleaving DNAzymes, RNA binding peptides, and DNA binding peptides and small molecules. RNA cleaving DNAzymes that lead to degradation of expanded CAG RNA have been shown to reduce polyQ protein levels across models of HD, SCA1, SCA3, SCA7, SCA17 and SSMA. Likewise, the CAG RNA binding peptide BIND, has been shown to reduce levels of polyQ proteins and CAG RNA foci in transfection models of SCA2, as well as rescuing a variety of downstream disease hallmarks in CAG expansion expressing Drosophila and transfection models of SCA2, SCA3 and HD. With these approaches, as with the CAG targeting ASO, expansion alleles are preferentially, but not exclusively targeted due to there being more binding sites for the therapeutic on expansion RNAs than on the equivalent non-expanded RNAs.

Furthermore, designer peptides that preferentially bind expanded CAG DNA reduce levels of HTT RNA and polyQ aggregates, as well as rescue behaviour phenotypes in HD models by interfering with transcription elongation.

Finally, in both HD and DRPLA mouse models, a CAG expansion DNA binding small molecule, naphthyridine-azaquinolone (NA), has been shown to reduce polyQ aggregates in the striatum by causing CAG repeat contractions. Interestingly, more recently, a dimer of NA (DoNA) has been shown to bind to CAG repeat RNA, however, the biological effects of DoNA are currently unknown. Together these studies demonstrate that multiple therapeutic strategies based on a variety of mechanisms can be used to realize therapeutic benefit and rescue multiple levels of the pathogenic cascade across multiple CAG expansion diseases.

Despite this, the potential of shared small molecule therapeutics that target the pathogenic source, the CAG repeat expansion, across CAG expansion SCAs remains under-studied with no small molecules currently published to reduce expression of CAG expansion transcripts in SCAs. To address this therapeutic development gap for this group of rare devastating diseases, we have designed a small molecule screening system to identify compounds that selectively regulate abundance of CAG RNAs in a disease-independent approach.

As such, there is an unmet need for SCA therapies that address underlying disease mechanisms. Here, we utilized the shared CAG repeat expansion mutation causative for a large subgroup of SCAs, to develop a novel disease-gene independent and mechanism agnostic small molecule screening approach to identify compounds with therapeutic potential across multiple SCA.

### BRIEF SUMMARY OF INVENTION

Disclosed herein are embodiments directed to a method for treating a subject having a CAG repeat expansion disorder, the method comprising: administering to the subject a therapeutically effective amount of a compound that selectively reduces the level of transcripts containing expanded CAG repeats, wherein the compound is a small molecule and is not an antisense oligonucleotide, short hairpin RNA, or microRNA. Embodiments disclosed herein are directed to a method for treating a subject having a CAG repeat expansion disorder, the method comprising: administering to the subject a therapeutically effective amount of the compound 4-[(4-methylphenyl)diazenyl]-1-phenylpyrazole-3,5-diamine or the compound colchicine or a derivative thereof, wherein the compound 4[(4-methylphenyl)diazenyl]-1-phenylpyrazole-3,5-diamine and derivatives bind to CAG repeat RNA. Further embodiments disclosed herein are directed to a method for treating a subject having a CAG repeat expansion disorder, the method comprising: administering to the subject a therapeutically effective amount of a compound selected from the group consisting of:

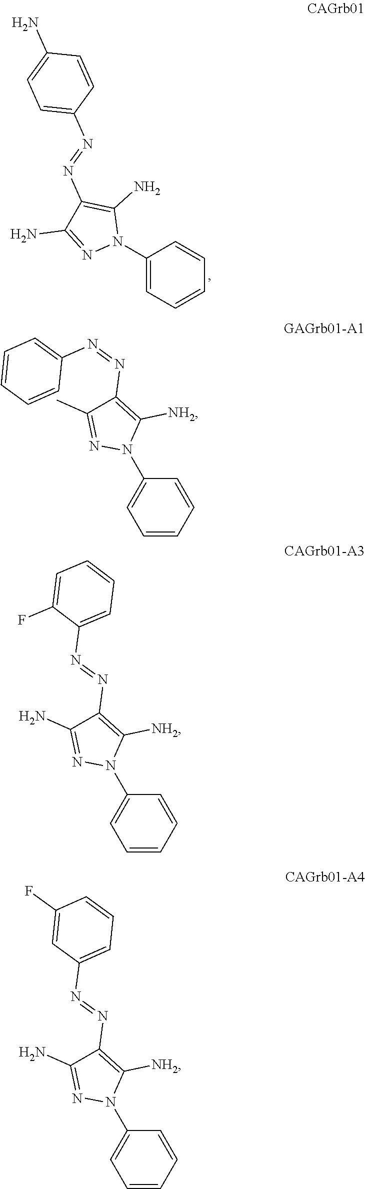 [SMILES: Nc1ccc(/N=N/c2c(N)nn(-c3ccccc3)c2N)cc1 ; Cc1nn(-c2ccccc2)c(N)c1/N=N\c1ccccc1 ; Nc1nn(-c2ccccc2)c(N)c1/N=N/c1ccccc1F ; Nc1nn(-c2ccccc2)c(N)c1/N=N/c1cccc(F)c1]

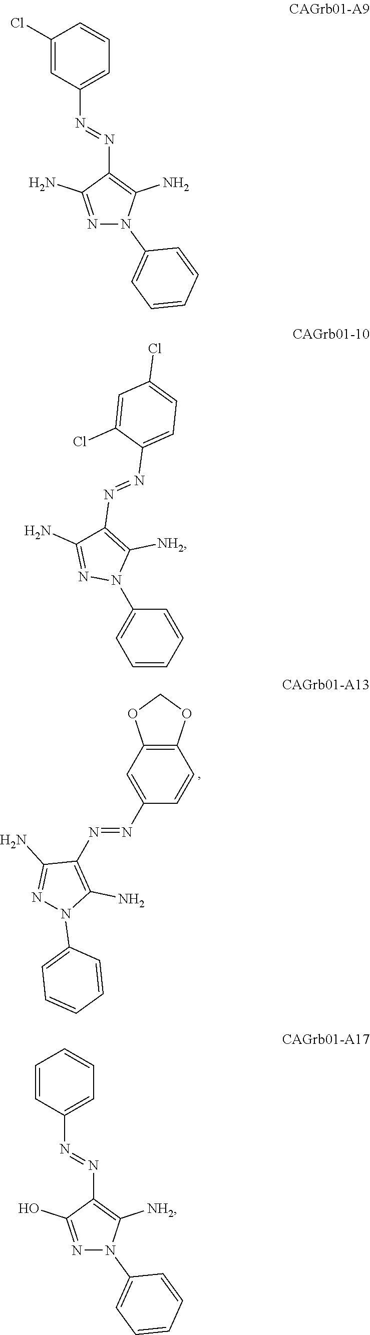

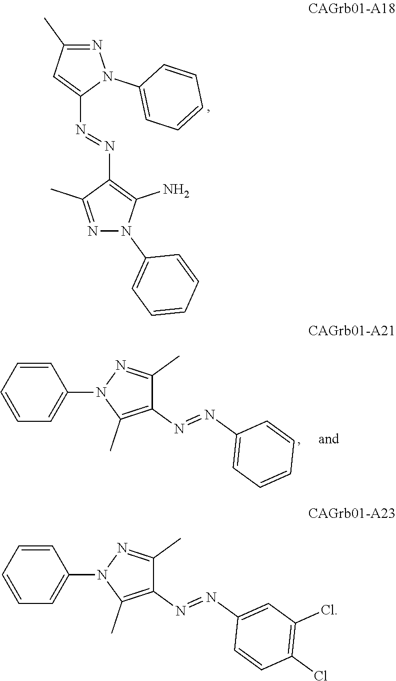

Additional embodiments disclosed herein are directed to the method for treating a subject having a CAG repeat expansion disorder, wherein the CAG repeat expansion disorder is selected from the group consisting of: spinocerebellar ataxia type 1 (SCA1), type 2 (SCA2), type 3 (SCA3), type 6 (SCA6), type 7 (SCA7), type 8 (SCA8), type 17 (SCA17), Huntington's disease, dentatorubral-pallidoluysian atrophy (DRPLA), and spinal and bulbar muscular atrophy (SBMA). Further embodiments are directed to the method for treating a subject having a CAG repeat expansion disorder, further comprising the step of assessing alternative splicing correction in the subject, wherein the compound rescues dysregulated splicing caused by CAG repeat expansion. (00153 Embodiments disclosed herein are directed to a method for identifying a therapeutic compound for treating a CAG repeat expansion disorder, wherein the method comprises: (a) providing a cell expressing a transcript comprising an expanded CAG repeat; (b) contacting the cell with a test compound; (c) measuring the level of the transcript comprising the expanded CAG repeat in the cell; and (d) identifying the test compound as a candidate therapeutic if the level of the transcript is selectively reduced compared to a control, wherein the test compound is not an antisense oligonucleotide. Embodiments herein are directed to the method for identifying a therapeutic compound for treating a CAG repeat expansion disorder, wherein the compound is

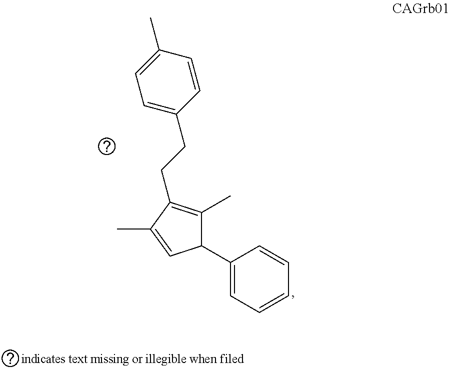 [SMILES: CC1=CC(c2ccccc2)C(C)=C1CCc1ccc(C)cc1 ; ","]

or wherein the compound is selected from the group consisting of:

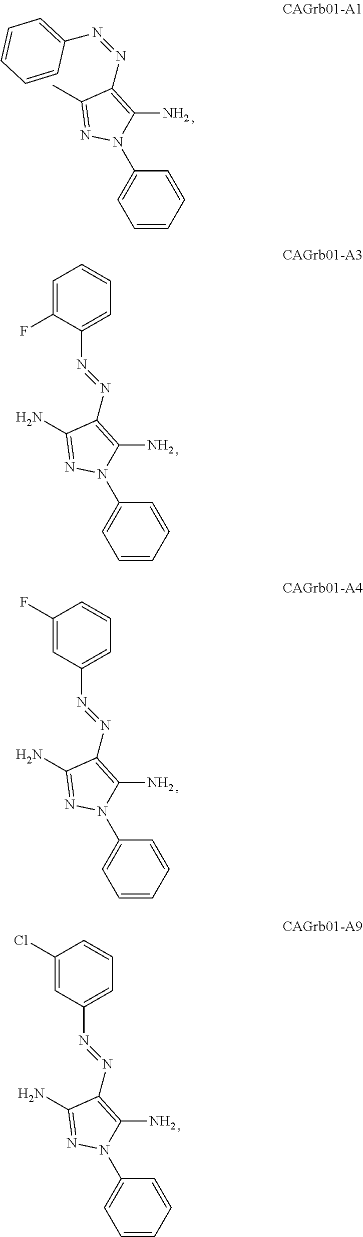 [SMILES: Cc1nn(-c2ccccc2)c(N)c1/N=N\c1ccccc1 ; Nc1nn(-c2ccccc2)c(N)c1/N=N/c1ccccc1F ; Nc1nn(-c2ccccc2)c(N)c1/N=N/c1cccc(F)c1 ; Nc1nn(-c2ccccc2)c(N)c1/N=N/c1cccc(Cl)c1]

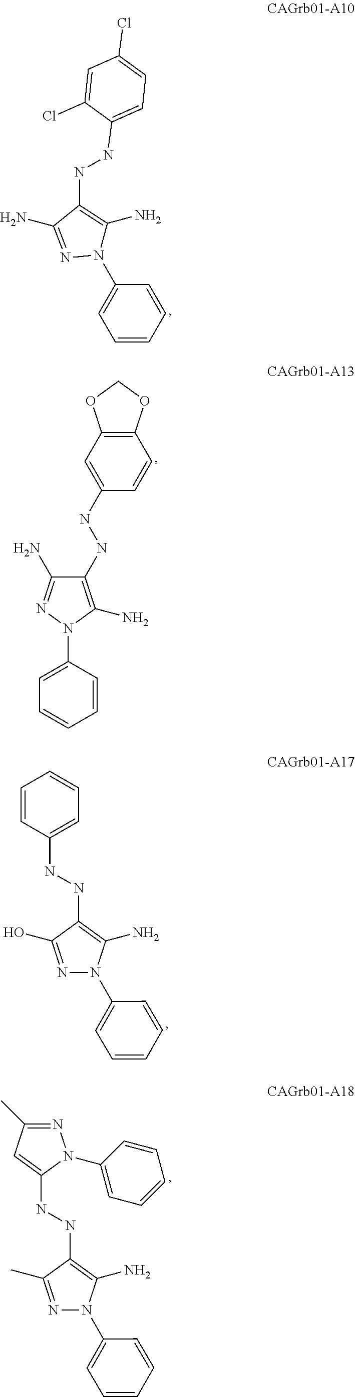

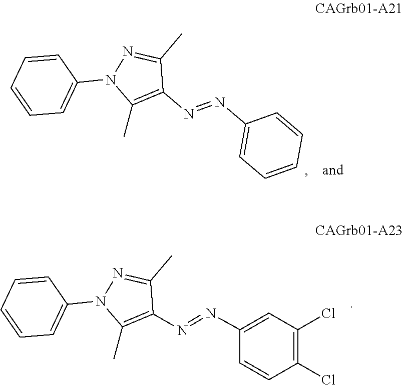 [SMILES: Cc1nn(-c2ccccc2)c(C)c1/N=N/c1ccccc1 ; Cc1nn(-c2ccccc2)c(C)c1/N=N/c1ccc(Cl)c(Cl)c1]

An embodiment disclosed herein is directed to a pharmaceutical composition comprising, a small molecule that binds to CAG repeat RNA and selectively reduces expanded CAG transcript levels; and a pharmaceutically acceptable carrier, wherein the small molecule is identified using methods disclosed herein.

Further embodiments are directed to a method for identifying compounds that selectively reduce the expression of CAG repeat-containing RNA and associated polyglutamine proteins, wherein the method comprises culturing a mammalian cell line co-expressing: a first reporter construct comprising at least 60 CAG trinucleotide repeats operably linked to a reporter gene encoding a nanoluciferase-polyglutamine fusion protein; and a second reporter construct lacking CAG repeats and encoding a firefly luciferase control protein; contacting the cell line with a candidate compound; measuring expression levels of: (i) the CAG repeat-containing RNA using RT-qPCR, and (ii) the polyglutamine-nanoluciferase fusion protein using a luciferase assay; identifying the compound as a selective modulator of CAG expansion RNA and polyglutamine protein expression if it reduces the expression of the CAG repeat-containing RNA by at least about 15%, and the expression of the polyglutarnine fusion protein by at least about 40%, compared to untreated or vehicle-treated controls. Some embodiments are directed to candidate compounds that are selected from the National Cancer Institute Diversity Set VI. Other embodiments are directed to compounds that are pyrazole-based compounds. Some embodiments are directed to the method's cell line, which is a HEK293T clone expressing integrated constructs via a PiggyBac transposon system. Embodiments are directed to the method for identifying compounds that selectively reduce the expression of CAG repeat-containing RNA and associated polyglutamine proteins, wherein the compound is 4[4-methylphenyl)diazenyl]-1-phenylpyrazole-3,5-diamine, or the compound is selected from the group consisting of:

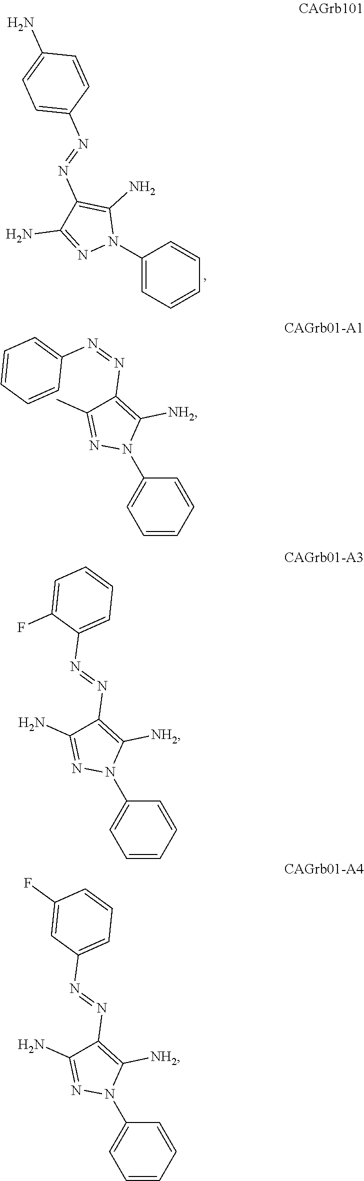 [SMILES: Nc1ccc(/N=N/c2c(N)nn(-c3ccccc3)c2N)cc1 ; Cc1nn(-c2ccccc2)c(N)c1/N=N\c1ccccc1 ; Nc1nn(-c2ccccc2)c(N)c1/N=N/c1ccccc1F ; Nc1nn(-c2ccccc2)c(N)c1/N=N/c1cccc(F)c1]

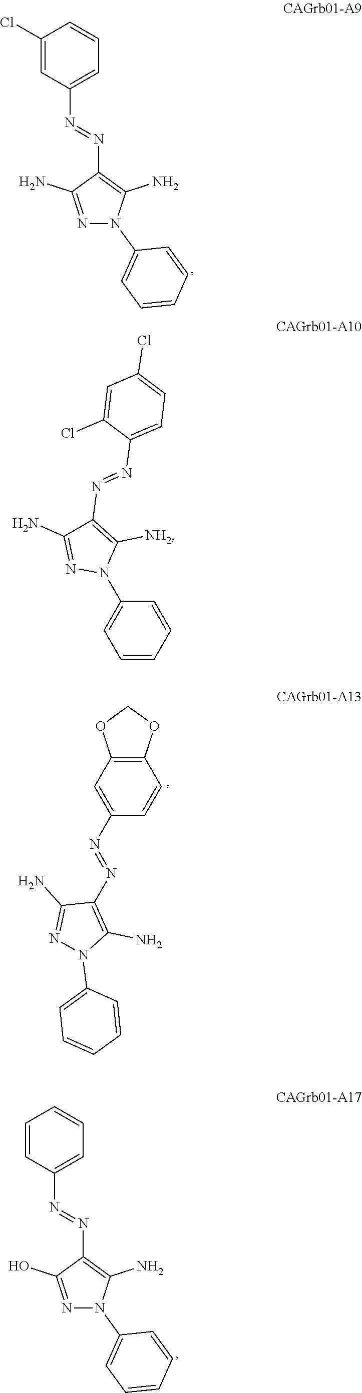

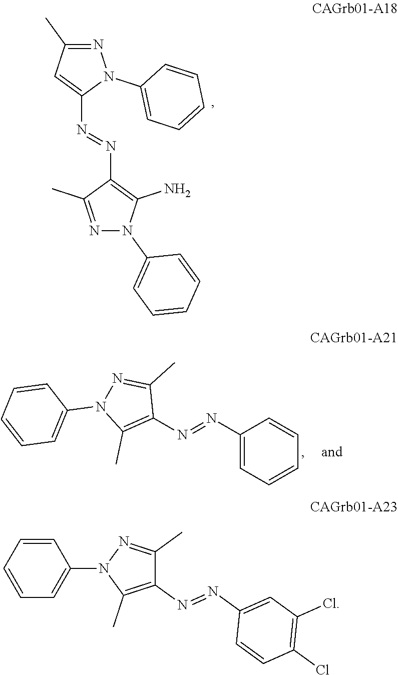 [SMILES: Cc1cc(/N=N/c2c(C)nn(-c3ccccc3)c2N)n(-c2ccccc2)n1 ; Cc1nn(-c2ccccc2)c(C)c1/N=N/c1ccccc1 ; Cc1nn(-c2ccccc2)c(C)c1/N=N/c1ccc(Cl)c(Cl)c1]

Further embodiments are directed a cell-based screening system for identifying compounds that modulate expression of CAG repeat-containing RNA, comprising: eukaryotic host cel line co-expressing: a first reporter construct comprising an open reading frame encoding a polyglutamine-nanoluciferase fusion protein, wherein the open reading frame includes at least 60GAG repeats; and a second reporter construct comprising an open reading frame encoding firefly luciferase, wherein the open reading frame includes 0 CAG repeats; wherein each reporter construct comprises a unique probe-binding sequence located downstream of the CAG repeat region; wherein the expression levels of the first and second reporter constructs are independently quantifiable via both: i) a dual luciferase assay, and ii) a multiplex RT-qPCR assay using fluorescent probes directed to the unique probe-binding sequences; and wherein the cell line is adapted for high-throughput screening of candidate therapeutic compounds targeting CAG repeat expansion disorders. Further embodiments are directed to a cell-based screening method for identifying CAG RNA-modulating compounds, comprising: (a) culturing the aforementioned cell line; (b) administering test compounds at concentrations≤10 μM; (c) quantifying (CAG)₆₀, and (CAG)₀ RNA levels via multiplex qPCR; and (d) selecting compounds showing≥20% reduction in (CAG)₆₀/(CAG)₀ RNA ratio with Z′ score≥0.51.

Other embodiments disclosed herein are directed to an engineered cell line for identifying compounds that modulate CAG repeat-containing RNA expression, comprising: (a) a first integrated nucleic acid construct encoding (CAG)₆₀-Myc-NLuc under an EF1 promoter; (b) a second integrated nucleic acid construct encoding (CAG)₀-Myc-FLuc under the EF1 promoter; (c) a puromycin resistance gene (Puro-R) and bidirectional stop cassettes (6xStop) flanking each construct to prevent readthrough transcription, and wherein the constructs are integrated into HEK293T cells to generate a plurality of clones that exhibit: (a) (CAG)₆₀ RNA expression and (CAG)₀ RNA, as measured by a multiplex qPCR comprising FAM- and HEX-labeled probes; (b) Z′ scores≥20.5 in RNA reduction assays; and (c) differential expression of SCA-associated genes consistent with CAG expansion disease models.

In certain embodiments, the invention provides a method for treating a subject having a CAG repeat expansion disorder by administering a therapeutically effective amount of a small molecule compound that selectively reduces the level of transcripts containing expanded CAG repeats. Notably, the compounds used in the disclosed methods are not antisense oligonucleotides, short hairpin RNAs, microRNAs or peptides. This approach offers a novel and targeted therapeutic strategy for modulating pathogenic transcript levels in CAG repeat-associated diseases, potentially improving clinical outcomes while avoiding the limitations associated with nucleic acid-based therapeutics.

### BRIEF DESCRIPTION OF THE OF THE DRAWING

FIG. 1A presents a schematic of constructs integrated into HEK293T genome to generate ATG-(CAG)60-Myc-NLuc/ATG-(CAG)O-Myc-Fluc cell line; Puro-R: puromycin resistance gene; EF1: human EF1 promoter; 6xStop: 2 stop cassettes in each reading frame. FIG. 1B presents the expression of (CAG)60 and (CAG)0 in parental HEK293T cells and clones 10, 15 and 37detected measured using FAM and HEX labelled probes respectively, in a multiplex qPCR; C(t):cycle threshold. FIG. 1C presents a dual luciferase assay for protein expression for polyQ-NLuc and FLuc in parental HEK293T cells and clones 10, 15 and 37. FIG. 1D presents protein blotting for polyQ-Myc-NLuc and Myc-FLuc in parental HEK293T cells and clones 10, 15 and 37; Arrow heads indicate FLuc, black bars indicate polyQ-NLuc,*-TATA binding protein. FIG. 1E presents a cell number fold change for parental HEK293T cells and clones, 10, 15 and 37; n≥4 per cell line per timepoint, mean*SEM, ** P<0.01, one-way ANOVA with Tukey's multiple comparisons test for day 4. FIG. 1F presents relative cell toxicity normalized to cell viability for parental HEK293T cells and clones 10, 15 and 37; n=6, mean t SEM; P<0.01, ** P<0.001, “ ” P<0.0001, one-way ANOVA with Tukey's multiple comparisons test. FIG. 1G presents a summary gene ontology terms identified using Metascape. FIG. 1H and FIG. 1I present RNA-Seq log 2fold change (log 2FC) (FIG. 1H) and qPCR validation (FIG. 1I) for seven genes significantly differentially expressed in a consistent direction in clone 15 and 37 compared to parental cell lines, and in CAG SCA mouse models. FIG. H Log2FC>0.5 (indicated by dashed line), Padj<0.05, n=3. I n=6, mean±SEM, one-way ANOVA with Tukey's multiple comparisons test, ns—not significant, * P<0.01, **** P<0.0001. FIG. 1J and FIG. 1K present CAG targeting siRNA [si(CAG)7]selectively reduces levels of CAG expansion transcripts (FIG. 1J) and polyQ proteins (FIG. 1K) after 48 hours. FIG. 1J clone 15 n≥5, clone 37 n=3, mean±SEM; K n=3, meant±SD. “ ” p<0.0001, two-tailed unpaired t-test: Z′ scores between 0.5 and 1 indicate that the assay is suitable for small molecule screening.

FIG. 2A and FIG. 2B Clone 37 (FIG. 2A) and clone 15 (FIG. 2B) show a reduction in (CAG)60 levels relative to (CAG)0 following 0.1 nM colchicine treatment; n=3, mean±SEM, two-tailed unpaired t-test, * P<0.05, “ ” P<0.0001; Z′ scores between 0.5 and 1 indicate that the assay is suitable for small molecule screening32. FIG. 2C presents relative cell viability of parental, clone 15 and clone 37 cells following 24 hours colchicine treatment compared to DMSO; dotted lines indicate 100% and 80% cell viability, n≥3, mean±SD. FIG. 2D through FIG. 2G present colchicine reduction of expression of ATXN1 transcripts in SCA1 patient-derived fibroblasts (FIG. 2D), ATXN7 in SCA7 fibroblasts (FIG. 2E), and ATXN3 in two SCA3 fibroblast lines (FIG. 2F, FIG. 2G); n=3, mean±SEM; unpaired two-tailed t-test (FIG. 2D), one-way ANOVA with Tukey's multiple comparisons test (FIG. 2E-FIG. 2G), *P<0.05, P<0.01. FIG. 2H through FIG. 2J colchicine does not affect expression of ATXN1 or ATXN3 in three control fibroblast lines and increases expression of ATXN7 in two out of three control fibroblast lines at doses which reduce expression of equivalent expansion alleles, see FIG. 2D through FIG. 2G; n=3, mean±SEM, unpaired two-tailed t-test, ns—not significant, * P<0.05, ** P<0.001. FIG. 2K presents relative cell viability of three control, one SCA1, two SCA3 and one SCA7 fibroblast lines following 48 hours treatment with colchicine; dotted lines indicate 100% and 80% relative viability; n=4, mean±SD, two-way ANOVA with Tukey's multiple comparisons test; Control 2 shows a significant reduction in viability at 10 nM compared to DMSO: ** P<0.01; all other cell lines show no significant difference between DMSO and 10 nM.

FIG. 3A is a 24 hour treatment of clone 37 with 0.1 nM colchicine selectively reduces (CAG)60 expression (normalized to (CAG)0); n=40 (n=2 per screening plate), mean±SEM, unpaired two-tailed t-test, **** P<0.0001. FIG. 3B presents a diversity set VI screen results for each individual compound treatment (1 μM in 0.01% DMSO) on (CAG)60 normalized to (CAG)Q for clone 37. Dashed grey line indicates DMSO average and dashed red line indicates colchicine average from FIG. 3A. Compounds that passed screening threshold are shown in red. FIG. 3C and FIG. 3E present a 24 hour treatment of clone 37 (C) and clone 15 (E) with si(CAG)7 selectively reduces polyQ-NLuc expression (normalized to FLuc); n=12 (n=2 per screening plate), mean±SEM, unpaired two-tailed t-test, * P<0.01, **** P<0.0001. FIG. 3D and FIG. 3F screen results for each individual compound treatment in clone 37 (FIG. 30) and clone 15 (FIG. 3F) on polyQ-NLuc normalized to FLuc, for the 72 hits identified in FIG. 3B. Dashed grey line indicates DMSO average and dashed red line indicates si(CAG)7 average from FIG. 3C or FIG. 3E for done 37 and clone 15, respectively. The three compounds that pass threshold in FIG. 3F are referred to, from left to right, as Hit 1, 2 and 3. Lead candidate Hit 2 (IUPAC name: 4-[(4-methylphenyl)diazenyl]-1-phenylpyrazole-3,5-diamine; CAS number: 5456-92-8) is shown in red; n=3, mean±SD. FIG. 3G Chemical structure and CAS number for Hit 2; IUPAC name: 4-[(4-methylphenyl)diazenyl)-1-phenylpyrazole-3,5-diamine; molecular formula: C₁₆H₁₆N₆; molecular weight: 292.34 g/mol. FIG. 3H presents the relative cell viability of parental, clone 15 and clone 37 cells following 24 hours Hit 2 treatment normalized to DMSO per cell line; dotted lines indicate 100% and 80% cell viability, clone 15 and clone 37 n=6, parental n≥3, mean±SD, one-way ANOVA with Tukey's multiple comparison test, ** P>0.001. FIG. 31 and FIG. 3K are 24 hour treatment of clone 15 (FIG. 31) and clone 37 (FIG. 3K) with Hit 2 in the tolerated dose range, reduces (CAG)60 expression (normalized to (CAG)0); clone 15 n=6, clone 37 n=3; mean±SEM, one-way ANOVA with Tukey's multiple comparisons test, ns—not significant, * P<0.05, ** P<0.01. FIG. 3J and FIG. 3L are 24 hour treatment of clone 15 (FIG. 3J) and clone 37 (FIG. 3L) with Hit 2 in the tolerated dose range, selectively reduces polyQ-NLuc expression (normalized to FLuc); n=6, mean±SD, one-way ANOVA with Tukey's multiple comparisons test, ns—not significant, *** P<0.001, **** P<0,0001.

FIG. 4A, FIG. 4B, and FIG. 4C are 24 hour treatment of ATG-(CAG)60-Myc-NLuc and ATG-(CAG)0-Myc-FLuc co-transfection in HEK293T cells with si(CAG)7 (FIG. 4A), or Hit 2 (FIG. 4C) in the tolerated dose range, reduce (CAG)60 expression (normalized to (CAG)0), but treatment with colchicine (FIG. 46) in the tolerated dose range does not affect (CAG)60 expression. A n=4, unpaired two-tailed t-test; FIG. 4B. FIG. 4C DMSO n=6, colchicine and Hit 2 n=3, one-way ANOVA with Tukey's multiple comparisons test, mean±SEM, ns—not significant, * P<0.05. *** P<0,0001. D 24 hour treatment of ATG-(CAG)60-Myc-NLuc and ATG-(CAG)0-Myc-FLuc co-transfection in HEK293T cells with Hit 2 in the tolerated dose range, selectively reduces polyQ-NLuc expression (normalized to FLuc); n≥4, mean±SD, one-way ANOVA with Tukey's multiple comparisons test, *** P<0.0001. E, F UV melting curves of r(CAG)10 at 2 μM with increasing molar ratios of Hit 2 (FIG. 4E) and calculated average melting temperatures (from FIG. 4E and FIG. 27J) for molar ratios of 0, 2.5, 4 and 5 (FIG. 4F); n=4, mean±SD, one-way ANOVA with Tukey's multiple comparisons test, ns—not significant, * P<0.05. FIG. 4G, FIG. 4H Fluorescent indicator displacement assay of 1 μM r(CAG)10 with 100 nM SYBR Safe showing displacement of SYBR Safe with increasing concentrations of Hit 2 (FIG. 4G) and (FIG. 4H) the average calculated dissociation constant (Kd; from FIG. 4G and FIG. 27K) in nM; n=3, mean±SEM. FIG. 4I, FIG. 4J, and FIG. 4K are computational modelling of a CAG repeat RNA containing a CAG helical region and a UUCG loop (from PBD ID: 7012) (FIG. 4I) and a CUG RNA duplex (from PBD 10: 1ZEV) (FIG. 4K) with Hit 2 binding to the minor groove, and modelling of CAG repeat RNA (from PBD ID: 7D12) enabling Hit 2 docking as an intercalator (FIG. 4J). Magnified site view in FIG. 4I and FIG. 4J highlight hydrogen bonding (dotted yellow lines) between Hit 2 amine groups and AA stacked mismatch and backbone (FIG. 4I) and (FIG. 4J) Hit 2 amine groups and A and C bases. L Docking scores representing binding affinity indicate Hit 2 binds more efficiently to CAG repeat RNA than CUG repeat RNA; thick line indicates median.

FIG. 5A presents relative cell viability of three control, one SCA1, two SCA3 and one SCA7 fibroblast lines following 48 hours treatment with Hit 2; dotted lines indicate 100% and 80% relative viability; n=4, mean±SD, two-way ANOVA with Dunnett's multiple comparisons test; Control 2 shows a significant reduction in viability at 25 μM compared to DMSO: ** P<0.01; all other cell lines show no significant difference between DMSO and 25 μM. B-D Hit 2 reduces expression of ATXN1 transcripts in SCA1 patient-derived fibroblasts FIG. 58, and ATXN3 in two SCA3 fibroblast lines (FIG. 5C and FIG. 5D); n=3, mean±SEM; one-way ANOVA with Tukey's multiple comparisons test, * P<0.05, ** P<0.01. FIG. 5E-FIG. 5H present protein blotting and quantification of ATXN1 (FIG. 5E and FIG. 5F) and ATXN3 (FIG. 5G and FIG. 5H) protein levels from patient derived SCA1 (FIG. 5E and FIG. 5F) and SCA3-2 (FIG. 5G and FIG. 5H) fibroblast lines following 48 hours treatment with DMSO or Hit 2 at the indicated concentration, n=3; FIG. 5E and FIG. 5G mean±SD, unpaired two-tailed t-test, * P<0.05, ** P<0.01. FIG. 5I and FIG. 5J Hit 2 does not affect expression of ATXN1 in two out of three control fibroblast lines or of ATXN3 in three control fibroblast lines at doses which reduce expression of equivalent expansion alleles (see FIGS. 5B, 5C, and 5D); n=3, mean±SEM, FIG. 5I unpaired two-tailed t-test, FIG. 5J one-way ANOVA with Tukey's multiple comparisons test, ns—not significant, * P<0.05.

FIG. 6A presents Hit 2 as well tolerated in vivo and reduces expression of CAG expansion transcripts in the Atxn1154Q/2Q SCA1 mouse model A, B Weight (g) (FIG. 6A) and clasping score (scored between 0 [no phenotype]and 3 [full clasp]) (FIG. 68) across the two week treatment regimen with baseline three days before initial injection; n=4, mean±SD, clasping score is an average of two independent investigators per mouse per timepoint. FIG. 6C hematoxylin and eosin staining of liver, kidney, spleen, cerebellum and hippocampus from DMSO and Hit 2 treated mice. Scale bars: liver and kidney 100 μm, spleen and cerebellum 200 μm, hippocampus 400 μm. FIG. 6D scoring of liver inflammation from H&E staining of DMSO and Hit 2 treated mice; inflammation score based on number of inflammatory foci per 5 fields, n=4. E GFAP immunohistochemisty from layers I-IV of frontal cortex, scale bar: 200 μm. FIG. 6F Hit 2 does not induce hepatotoxicity as assessed by a panel of 23 target genes; n=4, two-way ANOVA with Tukey's multiple comparison test, ns—not significant, * P<0.05, *** P<0.001, **** P<0.0001. FIGS. 6G, 6H, and 6I show that Hit 2 reduces expression of Atxn1 (G), specifically Abn11540 alleles (1) but not Atxn12Q (H) alleles in the Atxn1154Q/20 SCA1 mouse model following a two week IP injection regimen from 18 to 20 weeks of age; n=4, mean±SEM; FIG. 6G and FIG. 6I unpaired two-tailed t-test, FIG. 6H one-way ANOVA with Tukey's multiple comparison test, ns—not significant, * P<0.05, ** P<0.01, ***P<0.001. FIG. 6J and FIG. 6K standard curve of Hit 2 at known concentrations in 100% methanol and mouse brain lysed in 100% methanol (FIG. 6J) and calculated concentrations for samples in mouse brain lysate of known concentration based on the equation of the mouse brain lysate standard curve (FIG. 6K). FIG. 6L calculated concentrations of Hit 2 in cortex and midbrain lysate from Atxn1154Q/2Q-Hit 2 treated mice #421 and #430. Concentrations are reported as pg per mg of brain lysed. FIG. 6M Cropped HPLC trace (top) showing Hit 2 peak (retention time 5.4-5.6 min) for Atxn1154Q/2Q-Hit 2 mouse #421 which was collected following HPLC and subjected to LC/MS revealing a peak at m/z 293.1618 which corresponds to Hit 2 MW [292.346+H+]. For full HPLC trace and complete LC/MS see FIGS. 30B and C.

FIGS. 7A-7N presents lead candidate Hit 2 rescues alternative splicing dysregulation in the Atxn1154Q/2Q SCA1 knockin mouse model A, B PSI (FIG. 7A) and log2FC (FIG. 78) for WT-DMSO vs Atxn1154Q/20-DMSO (x-axis) and WT-DMSO vs Atxn11540/20-Hit 2 (y-axis) showing events significantly different between WT-DMSO vs Atxn1154Q/2Q-DMSO (□PSI>10%, FDR<0.1; log 2FC>1, padj<0.05; Phenotype—Blue) and WT-DMSO vs Atxn1154Q/2Q-Hit 2 (Off Target—Pink). Events that cluster around the x-axis show 100% rescue on Hit 2 treatment and events that cluster along the dotted line show no-rescue no Hit 2 treatment. FIG. 7C and FIG. 7D PCA plots based on phenotype events (see FIG. 6A and FIG. 6B) for differential gene expression (FIG. 7C) and alternative splicing (FIG. 7D) showing clear rescue of dysregulation of alternative splicing but not differential gene expression on Hit 2 treatment. FIG. 7E heatmap showing 20 genes differentially expressed between WT-DMSO and Atxn1154Q/2Q-DMSO (log 2FC>1, padj<0.05) and their response to Hit 2 treatment. FIG. 7F number of SE events rescued (>10% rescue and PSI>5% between SCA DMSO and Hit 2) and number of non-rescued SE events which include events showing an opposite effect (>10% change in splicing away from WT PSI with a minimum PSI of 5% between SCA DMSO and Hit 2) and events that are not-rescued (all remaining events). FIG. 7G and FIG. 7H is the percentage of exon inclusion (positive) or exclusion (negative) for events that show increased exon inclusion between WT-DMSO and Atxn11540/20-DMSO (FIG. 7G) and events that show increased axon exclusion between WT-DMSO and Atxn1154Q/2Q-DMSO (FIG. 7H) (both: FDR<0.1, PSI>10%) and the response to Hit 2 treatment, separated into rescued and non-rescued events (based on FIG. 6F), mean * SD. FIG. 7I heatmap showing SE events dysregulated between WT-DMSO and Atxn1154Q/20-DMSO (□PSI>10%, FDR<0.1) and their response to Hit 2 treatment for events significantly rescued by Hit 2 (Atxn1154Q/2Q-DMSO vs-Hit 2: □PSI>5%, FDR<0.1, >10% rescue). FIGS. 7J, 7K, 7L, and 7M present violin plots of RNA-Seq data for SE events significantly dysregulated between WT-DMSO and Atxn1154Q/2Q-DMSO and significantly rescued following Hit 2 treatment, showing that the increased exclusion of Atpl3a4 exon 24 (FIG. 7J) and Wnk1 exon 4b (FIG. 7K) and the increased inclusion of Zbtb20 axon 5 (FIG. 7L) and Cacna2d2 exon 23 (FIG. 7M) is rescued on Hit 2 treatment, line indicates median. FIG. 7N RT-PCR analysis of Cacna2d2 exon 23 showing rescue following Hit 2 treatment; n=4, one-way ANOVA with Tukey's multiple comparisons test, ns—not significant, * P<0.01, mean±SD.

FIG. 8 presents a comparison of Hit 2 with structural analog CAGrb01-A1 in CAG repeat expressing (i.e., reduction of expression of the resulting RNA and protein) screening cell line (i.e., clone All, a rederivation of clne 37 as more fully described herein below).

FIG. 9 presents a comparison of Hit 2 with structural analog CAGrb01-Alin patient derived fibroblast lines.

FIG. 10 presents analog CAGrb01-A1 binding affinity (Score in Kcal/mol) (S)=−5.419 Solubility (logS)=−3.342; and binds in the minor groove and preserves NH₂ interaction with the A section.

FIG. 11 presents analog CAGrb01-A3 binding affinity (Score in Kcal/mol) (S)=−5.802 Solubility (logS)=−3.791; and binds in the minor groove and preserves NH₂ interaction with the backbone.

FIG. 12 presents analog CAGrb01-A4 binding affinity (Score in Kcal/mol) (S)=−5.774 Solubility (logS)=−3.791; and binds in the minor groove and preserves NH₂ interactions with the base sidechain and the backbone.

FIG. 13 presents analog CAGrb01-A9 binding affinity (Score in Kcal/mol) (S)=−5.860 Solubility (logS)=−4.230; and binds in the minor groove and preserves NH₂ interaction with the A section.

FIG. 14 presents analog CAGrb01-A10 binding affinity (Score in Kcal/mol) (S)=−5.738 Solubility (logS)=−4.964; and binds in the minor groove and preserves NH₂ interaction with the backbone.

FIG. 15 presents analog CAGrb01-A13 binding affinity (Score in Kcal/mol) (S)=−5.989 Solubility (logS)=−3,451; and binds in the minor groove and preserves NH₂ interaction with the base sidechain.

FIG. 16 presents analog CAGrb01-A17 binding affinity (Score in Kcal/mol) (8)=−5.728 Solubility (logS)=−3,413; and binds in the minor groove and preserves NH₂ interaction with the backbone.

FIG. 17 presents analog CAGrb01-A18 binding affinity (Score in Kcal/mol) (S)=−6.008 Solubility (logS)=−4.556, and that it binds in the minor groove.

FIG. 18 presents analog CAGrb01-A21 binding affinity (Score in Kcal/mol) (S)=−4.673 Solubility (logS)=−4.058, and that it binds in the minor groove,

FIG. 19 presents analog Grb01-A23 Binding Affinity (Score in Kcal/mol) (S)=−4,861 Solubility (logS)=−5.527, and that it binds in the minor grove.

FIG. 20 presents analog CAGrb01-A6 binding affinity (Score in Kcal/mol) (S)=5.880 Solubility (logS)=−5.095; this analog does not include NH₂ hydrogen bond donor groups and contains carbonyl group which is predicted to act as a hydrogen bond acceptor in the interaction with CAG RNA helix.

FIG. 21 presents analog CAGrb01-A12 binding affinity (Score in Kcal/mol) (S)=−5.786 Solubility (logS)=−4.490; this analog contains carbonyl group which is predicted to act as a hydrogen bond acceptor in the interaction with CAG RNA helix.

FIG. 22 presents analog CAGrb01-A15 binding affinity (Score in Kcal/mol) (S)=−5.909 Solubility (logS)=−3.646; and this analog does not include NH₂ hydrogen bond donor groups Contains carbonyl group which is predicted to act as a hydrogen bond acceptor in the interaction with CAG RNA helix.

FIG. 23 presents analog CAGrb01-A20 binding affinity (Score in Kcal/mol) (S)=−6.041 Solubility (logS)=−6.888; and contains carbonyl group which is predicted to act as a hydrogen bond acceptor in the interaction with CAG RNA helix does not bind to the minor groove.

FIG. 24 presents analog CAGrb01-A22 binding affinity (Score in Kcal/mol) (S)=−5.007 Solubility (logS)=−5.527; and this analog does not include NH₂ hydrogen bond donor groups and does not bind to the minor groove.

FIG. 25 presents analog CAGrb01-A24 binding affinity (Score in Kcal/mol) (S)=−4.264 Solubility (logS)=−2.340; and this analog does not include NH, hydrogen bond donor groups.

FIG. 26A and FIG. 26B Clone 15 (FIG. 26A) and clone 37 (FIG. 268) show more downregulated genes that upregulated genes; log 2FC>0.5. Padj<0.05, n=3. FIG. 26C clone 15 and 37 show differential expression of genes previously reported to be differentially expressed in CAG expansion SCA mouse models, log 2FC>0.5, Padj<0.05, n=3. FIG. 26D cycle threshold [C(t)J for GABRA3, ALDHIL2 and SLC7A11 which show an almost complete ablation of gene expression following analysis using the 2′ method; GAPDH shown as housekeeping gene; n=6, mean±SEM. FIG. 26E and FIG. 26F RNA-Seq log 2foid change (log 2FC) (FIG. 26E) and qPCR validation (FIG. 26F) for METTL15, included as a negative control for qPCR validation as this gene does not show differential expression on RNA-Seq analysis. FIG. 26E dashed line indicates log 2FC=0.5, n=3. FIG. 26F n=6, mean±SEM, one-way ANOVA with Tukey's multiple comparisons test, ns—not significant.

FIG. 27A and FIG. 27B colchicine does not reduce levels of polyQ-NLuc relative to FLuc in clone 37 (FIG. 27A) or clone 15 (FIG. 27B) at doses that result in a selective reduction of CAG expansion transcript levels; n=3, mean±SEM, one-way ANOVA with Tukey's multiple comparisons test, ns—not significant. FIG. 27C, FIG. 27E 24 hour treatment of clone 37 (FIG. 27C) and clone 15 (FIG. 27E) with CAG targeting siRNA selectively reduces (CAG)60 expression (normalized to (CAG)0); n=2 per screening plate, mean±SEM, unpaired two-tailed t-test, **** P<0.0001. FIG. 27D, FIG. 27F cycle threshold [C(t)J values for multiplex qPCR of DMSO negative control samples for reverse transcriptase (RT) reaction with RT enzyme (DMSO) and without RT enzyme in the reaction (DMSO RT-); n=2 per screening plate, mean±SEM. FIG. 27G 24 hour treatment of clone 15 with 0.1 nM colchicine selectively reduces (CAG)60 expression (normalized to (CAG)0); n=12 (n=2 per screening plate), mean±SEM, unpaired two-tailed t-test, “** P<0.0001. FIG. 27H presents compounds that passed the threshold for reduction of (CAG)60 expression in clone 15. FIG. 27I presents melting curve to higher temperatures. FIG. 27J presents average melting temperatures for molar ratios. FIG. 27K presents displaced approximately of SYBR Safe and concentration. FIG. 27I UV absorbance curves at 260 nm of increasing concentrations of Hit 2 from 0 μM to 10 μM (molar ratio of 0 to 5) with and without r(CAG)10 at 2 μM. FIG. 27J UV melting curves of r(CAG)10 at 2 μM with increasing molar ratios of Hit 2 (at ratios of 0, 2.5, 4 and 5) for three independent experiments. FIG. 27K Fluorescent indicator displacement assay of 1 μM r(CAG)10 with 100 nM SYSR Safe showing displacement of SYBR Safe with increasing concentrations of Hit 2 for two independent experiments.

FIG. 28A presents ATXN7 expression across multiple doses in three control fibroblast lines, and the screen results for each individual compound treatment in clone 15 on (CAG)60 normalized to (CAG)0, for the 72 hits are identified in FIG. 288. Dashed grey line indicates DMSO average and dashed red line indicates 0.1 nM colchicine average from FIG. 28E; lead candidate Hit 2 is shown in red; n=3, mean±SEM. Hit 2 does not rescue disease hallmarks in DM1 myotubes FIG. 28A, and FIG. 28B Hit 2 does not have a consistent effect on the expression of ATXN7 in three control fibroblast lines (A; n=3) or on ATXN7 in SCA7 patient derived fibroblast lines (B, n≥4). FIG. 28C three-day treatment of 1 μM Hit 2 increases expression of DMPK in DM1 patient derived myotubes; n=3, mean±SEM, one-way ANOVA with Tukey's multiple comparisons test, *P<0.05. FIG. 28D-FIG. 28F Hit 2 treatment does not affect splicing of hallmark skipped exon events in DM1 myotubes, n=3, mean±SD.

FIG. 29A, FIG. 29B mass spectrometry of Hit 2 sourced from the NCI DTP (FIG. 29A) and in house synthesized Hit 2 (FIG. 298). Top panels show total ion chromatograms and bottom panels show molecular ion peaks at 293.1729 (FIG. 29A) and 293.1979 (FIG. 298). FIG. 29C, FIG. 290 ¹H NMR (FIG. 29C) and ¹³C NMR (FIG. 29D) of in-house synthesized Hit 2.

FIG. 30A and FIG. 30B are representative traces of Hit 2 in 100% methanol, mouse cortex and midbrain lysate in 100% methanol, Hit 2 spiked into mouse cortex and midbrain lysate in 100% methanol (FIG. 30A) and lysate from cortex and midbrain of DMSO inject wild-type and Atxn1^{154Q/2Q} (FIG. 30B) and traces for the two Hit 2 injected Atxn1^{154Q/2Q} mice where peaks for Hit 2 were observed (mice #421 and #430; B). FIG. 30C LC/MS for sample retrieved from HPLC Hit 2 peak of mouse #421 revealing peak at m/z=293,1618 corresponding to Hit 2 MW: 292,346+H+.

FIG. 31A is a percentage of significantly misspliced skipped exon (SE), retained intron (RI), mutually exclusive exons (MXE), alternative 5′ splice site (A5SS) and alternative 3′ splice site (A3SS) events as a proportion of total splicing events in DMSO injected SCA1 vs WT mice, number of each event shown on bar, FDR<0.1, PSI>10%. FIG. 31B is a percentage of exon inclusion (positive) or exclusion (negative) for significantly alternatively spliced SE events, FDR<0.1. PSI>10%, mean±SD. FIG. 31C presents an enrichment of summary gene ontology terms identified using Metascape for the 495 SE events dysregulated in DMSO injected SCA1 mice compared to DMSO injected WT mice. FIG. 31D and FIG. 31E are violin plots of RNA-Seq data showing increased inclusion of Trpc3 exon 9 (FIG. 31D) and increased exclusion of Anks1b exon 5 (FIG. 31E) in SCA1 mice; FDR<0.1, PSI>10%, line indicates median. FIG. 31F RT-PCR analysis of Cacna2d2 exon 23 in cerebellum from presymptomatic (6 week, n=S), early symptomatic (12 week, n=4) and mild symptomatic (23 week, n=3) Atxn1^{154Q/2Q} SCA1 mice compared to WT; ns—not significant, ** P<0.001, unpaired two-tailed t-test, mean±SEM.

### DESCRIPTION OF THE INVENTION

Spinocerebellar ataxias (SCAs) are a genetically heterogenous group of devastating neurodegenerative conditions for which clinical care currently focuses on managing symptoms. Across these diseases there is an unmet need for therapies that address underlying disease mechanisms. Here, we utilized the shared CAG repeat expansion mutation causative for a large subgroup of SCAs, to develop a novel disease-gene independent and mechanism agnostic small molecule screening approach to identify compounds with therapeutic potential across multiple SCAs. Using this approach, we identified the FDA approved microtubule inhibitor Colchicine and a novel CAG-repeat binding compound that reduce expression of disease associated transcripts across SCA1, 3 and 7 patient derived fibroblast lines and the Atxn1^{154Q/2Q} SCA1 mouse model in a repeat selective manner. Furthermore, our lead candidate rescues dysregulated alternative splicing in Atxn1^{154Q/2Q} mice. This work provides the first example of small molecules capable of targeting the underlying mechanism of disease across multiple CAG SCAs.

This innovative disease-gene independent, pathway-agnostic approach has the potential to identify small molecules capable of alleviating the underlying disease mechanisms in CAG expansion SCAs as well as ameliorating downstream cellular consequences. Furthermore, by taking a pathway-agnostic approach, this screening platform has the capability of identifying compounds that regulate expression of expansion RNAs through a variety of mechanisms. Together, this system may therefore identify small molecules with therapeutic potential across multiple CAG expansion SCAs, and potentially across the broader range of CAG microsatellite expansion disorders.

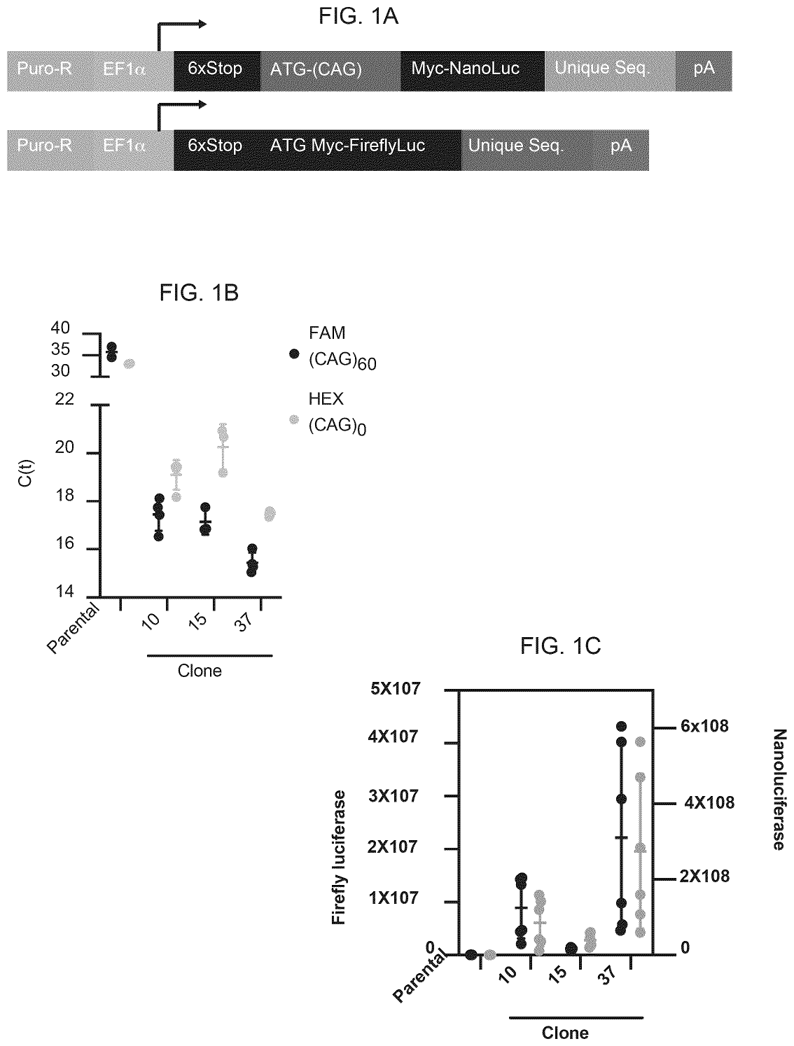

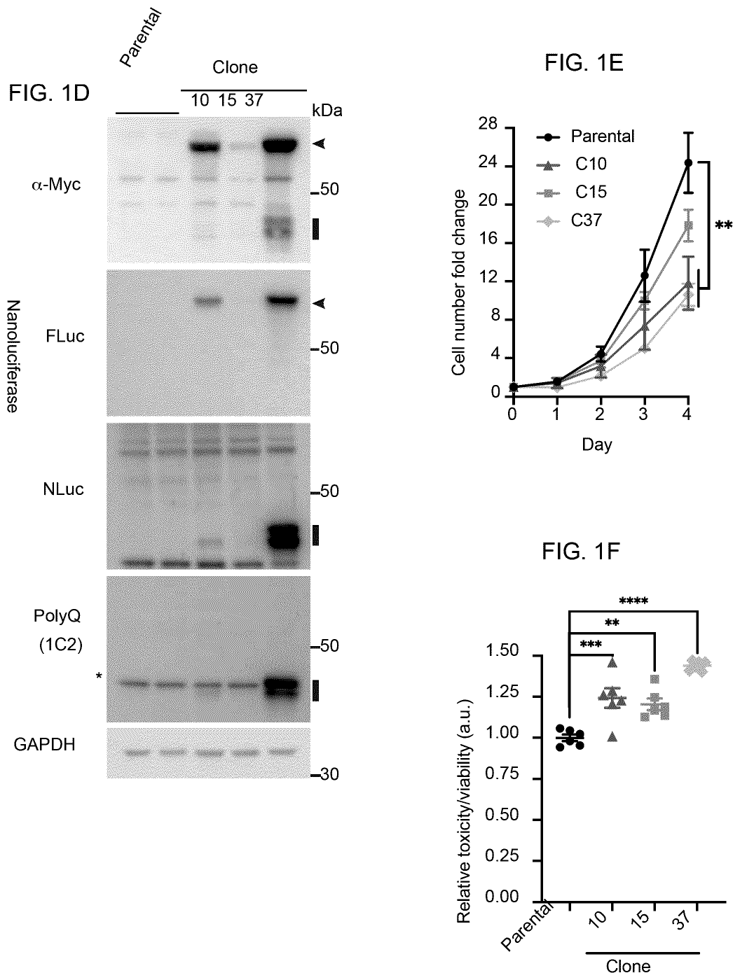

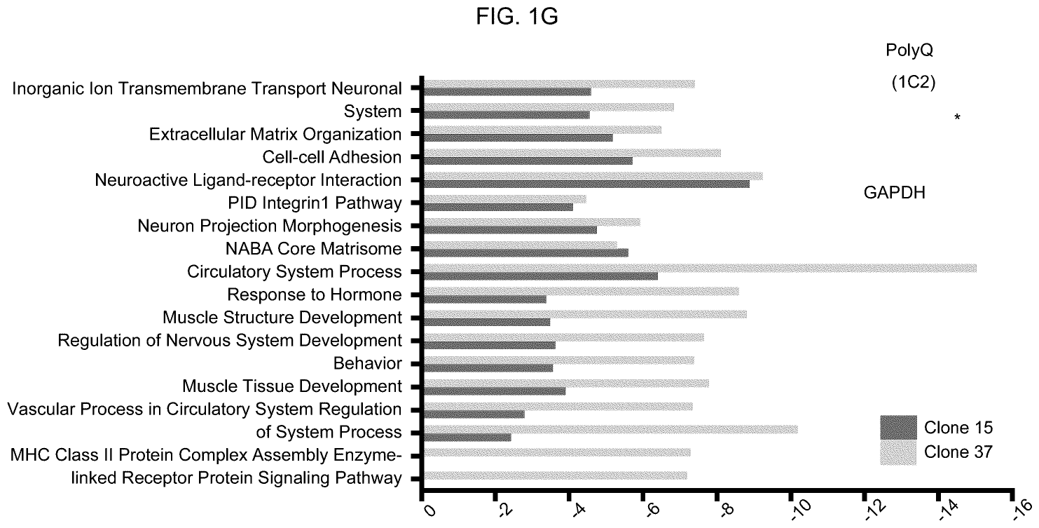

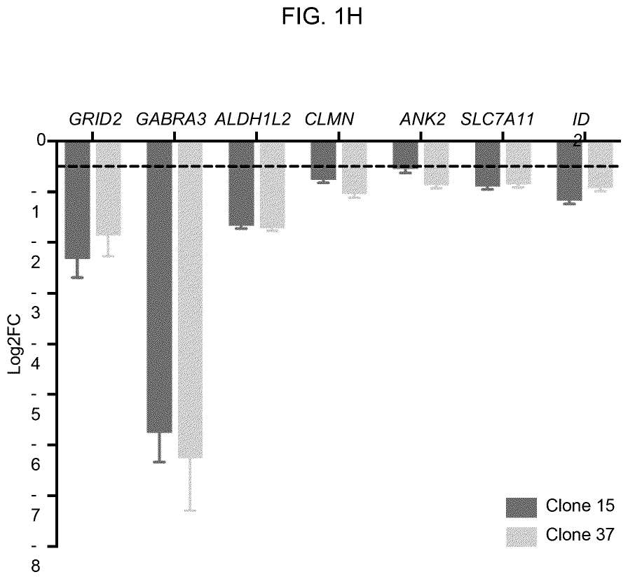

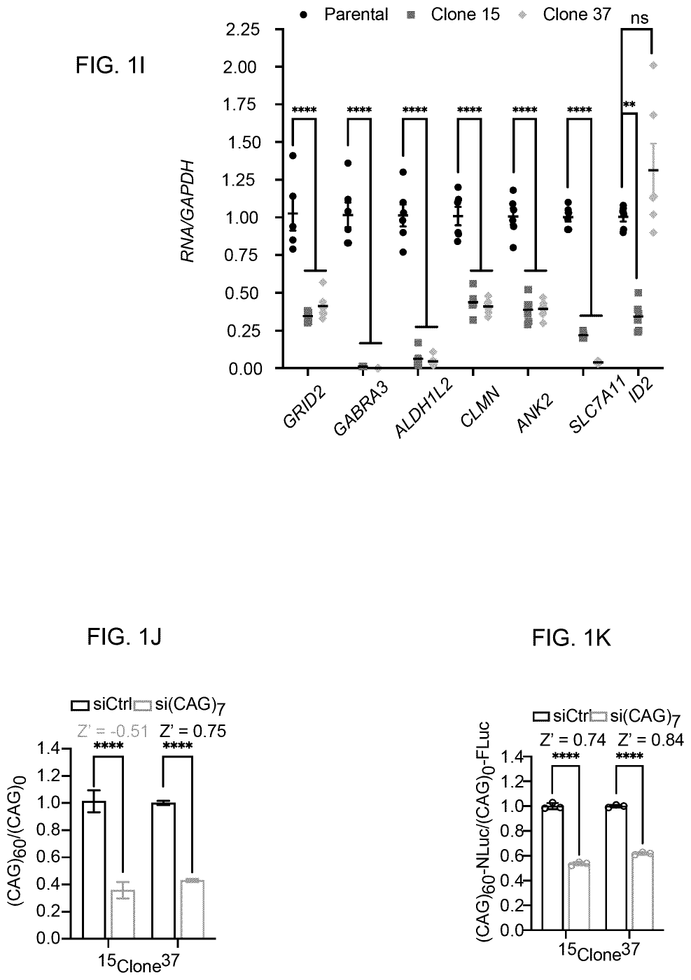

Generation and characterization of a HEK293T CAG repeat-selective screening system: Our central hypothesis is that regulating CAG expansion transcript abundance with small molecules will ameliorate downstream cellular consequences and yield therapeutic potential across multiple SCAs. To identify compounds capable of this, we established a CAG expansion-selective screening cell line, based on a similar system we previously reported for DM122. This screening system expresses ATG-(CAG)60-Myc-NLuc and ATG-(CAG)0-Myc-FLuc transcripts, each with a unique qPCR probe binding site 3′ of the repeat tract to distinguish the two transcripts. This permits the sensitive ratio-metric measurement of polyglutamine-nanoluciferase (polyQ-NLuc) relative to firefly luciferase (FLuc) using a dual luciferase assay, and measurement of r(CAG)60 relative to r(CAG)0 using a multiplex RT-qPCR with one common primer pair and fluorescent probes against the unique sequences (FIG. 1A).

These constructs do not contain genetic context from any SCA disease causing genes. The constructs were randomly integrated into the HEK293T cell genome using the PiggyBAC Transposon system, cells were selected for puromycin resistance, single cell sorted and screened for expression of both integrands. Low, medium, and high expressing clones were identified, with expression of both integrands confirmed via qPCR (FIG. 1B), dual luciferase assay (FIG. 1C) and protein blotting using antibodies against the myc epitope tag, FLuc, NLuc and polyQ (FIG. 1D).

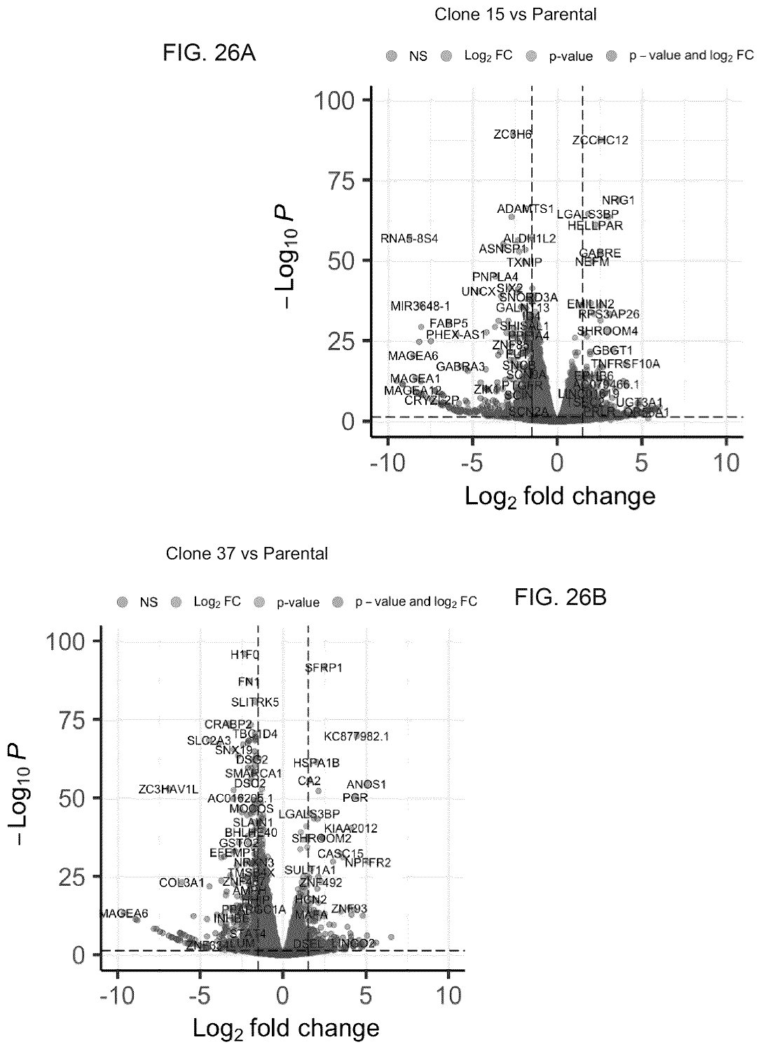

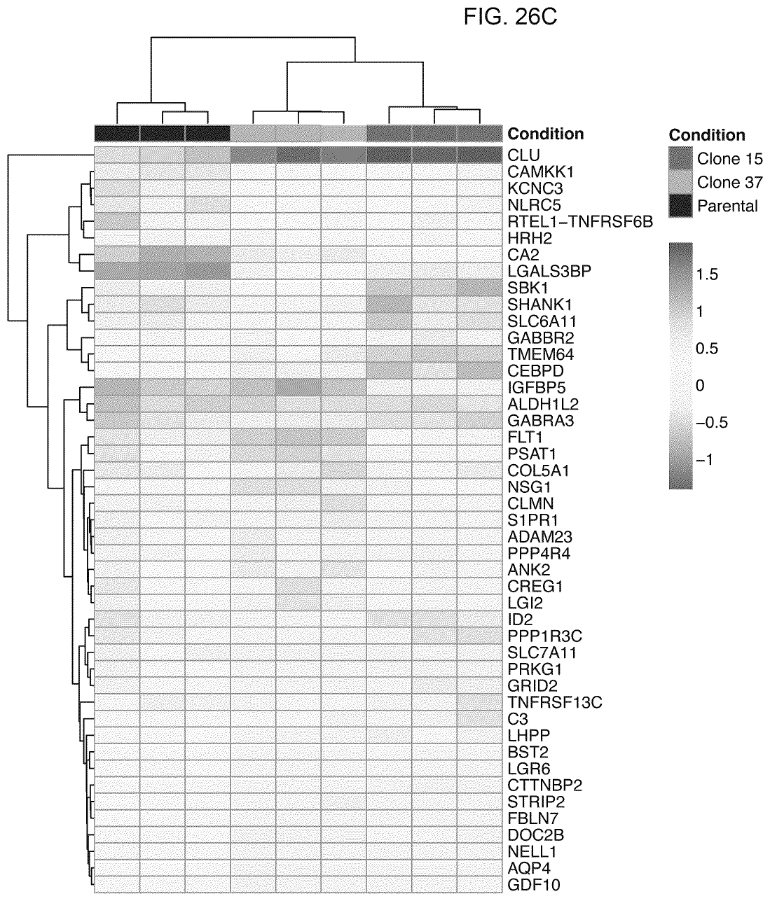

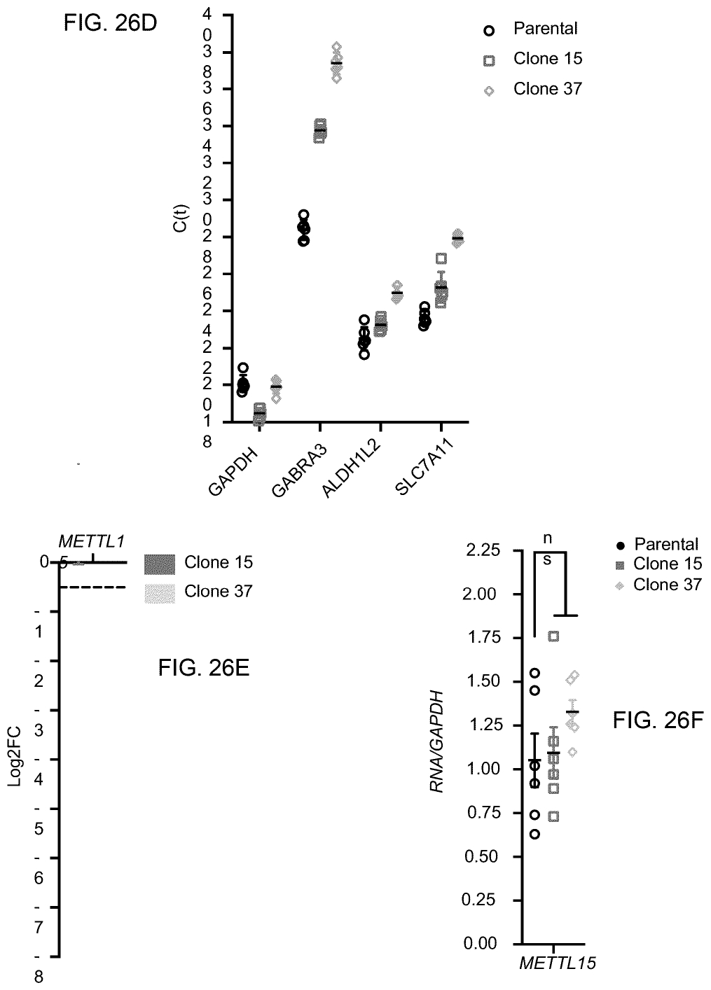

Upon characterization of the clonal cell lines, it was noted that all clonal lines grew slower than parental HEK293T cells, with clone 10 and clone 37 showing a 12.6% (P=0.0047) and 13.8% (P=0.0022) reduction in cell number fold change (growth rate) compared to parental four days after plating, respectively (FIG. 1E). This reduction in growth rate was accompanied by a 44.0% (P<0.0001) increase in relative cell toxicity for clone 37 compared to parental HEK293T cells. For clone 10 there was a 24.2% (P=0.0007) and for clne 15 a 20.4% (P=0.004) increase in cell toxicity compared to parental, thereby revealing a dose effect of increasing CAG repeat load with increasing cell toxicity. High and low expressing clones (clone 37 and clone 16) were selected for further characterization by RNA sequencing (RNA-Seq) analysis (Table S1). Compared to parental, both clonal lines demonstrated a greater number of downregulated genes compared to upregulated genes (FIGS. 26A, 26B) consistent with differential gene expression patterns seen in mouse models of CAG SCAs.

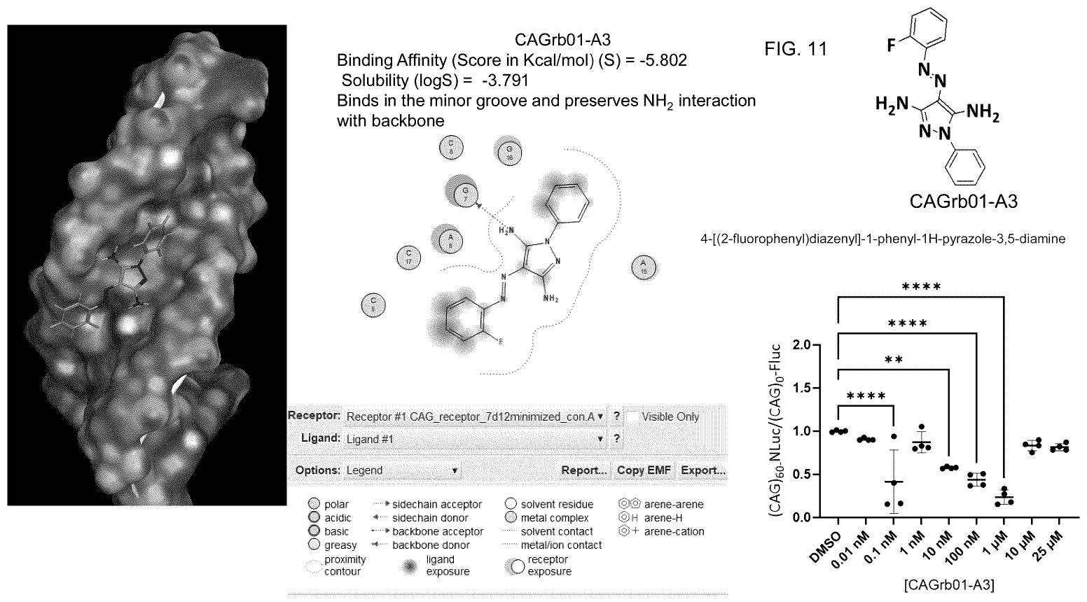

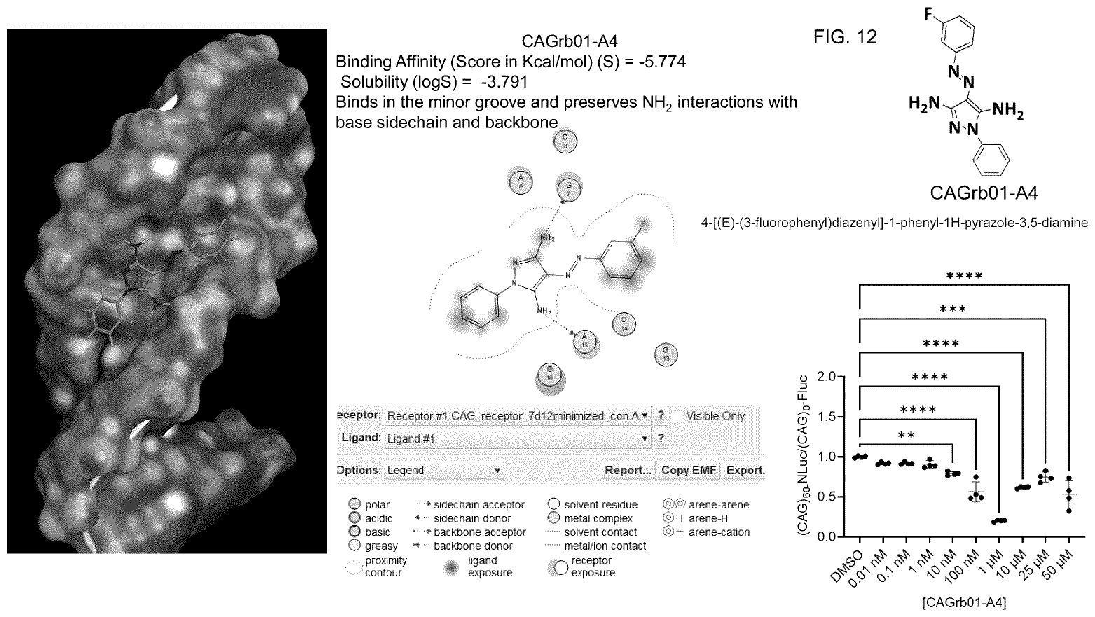

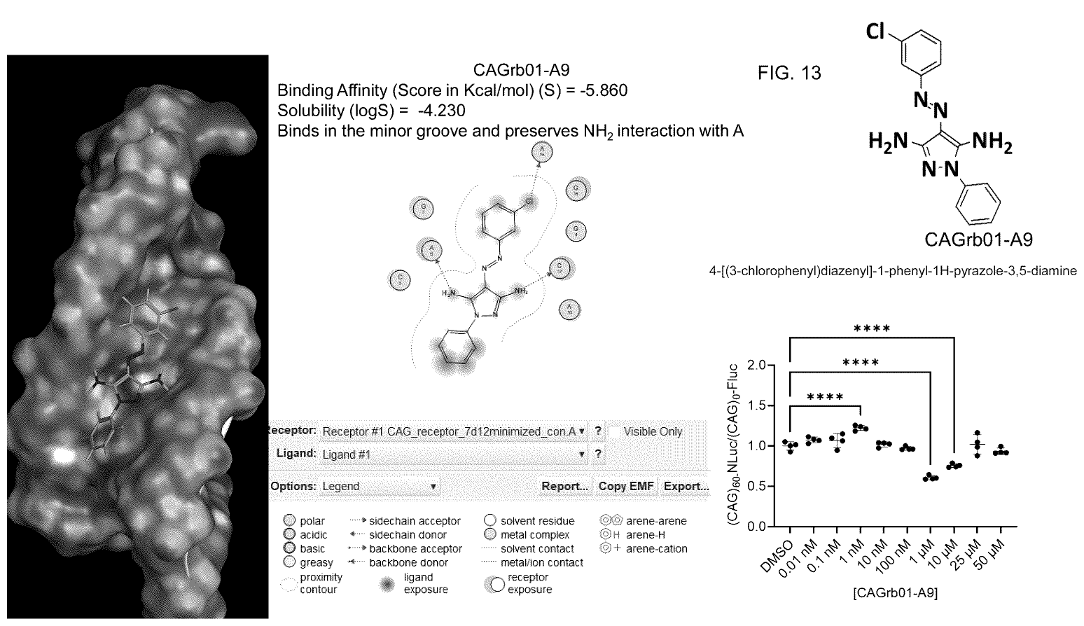

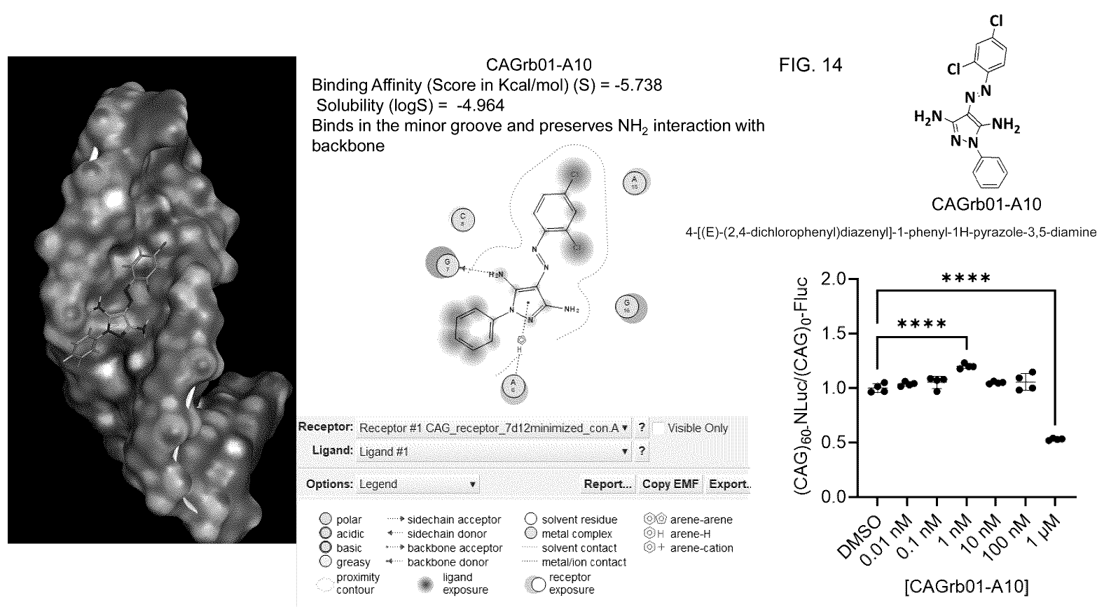

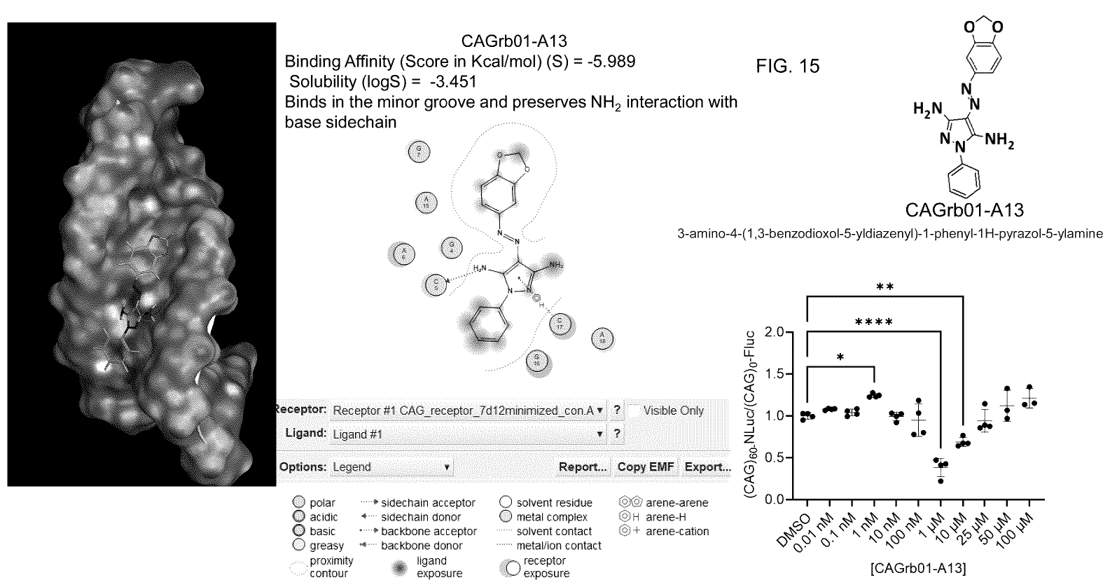

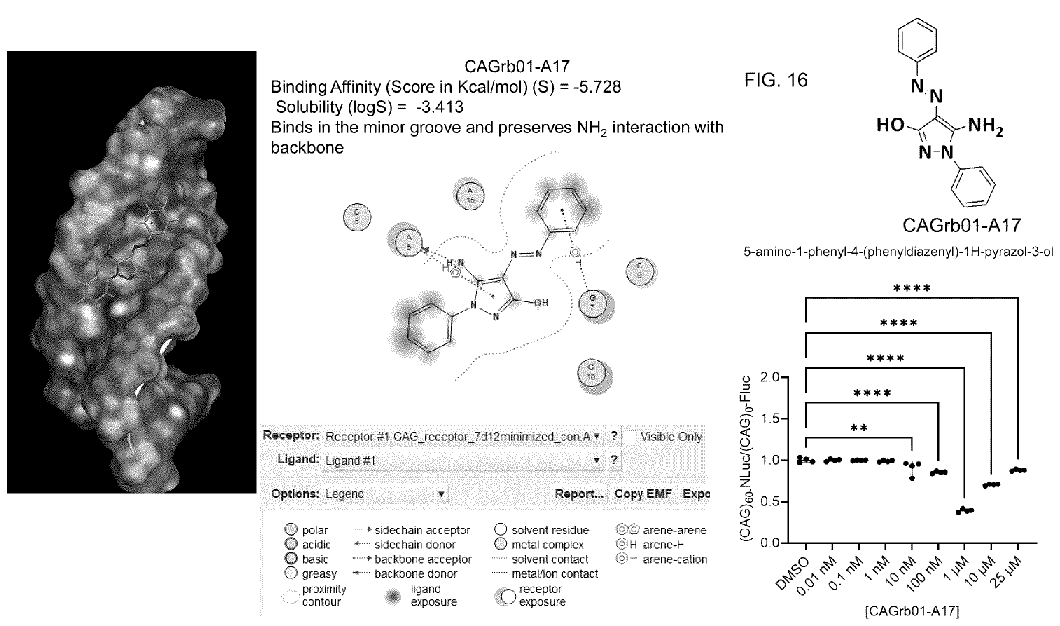

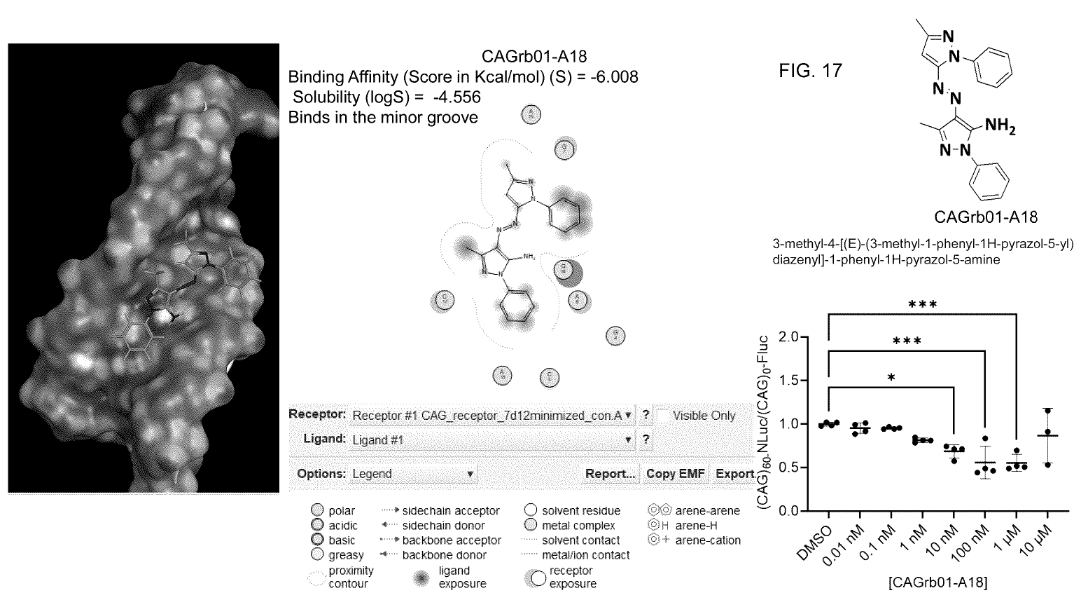

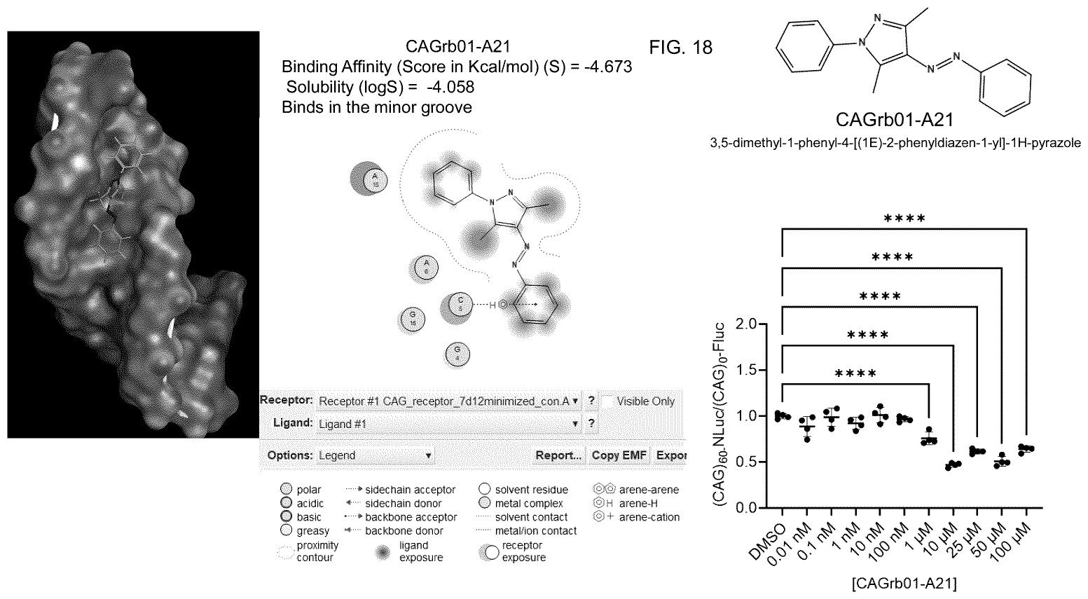

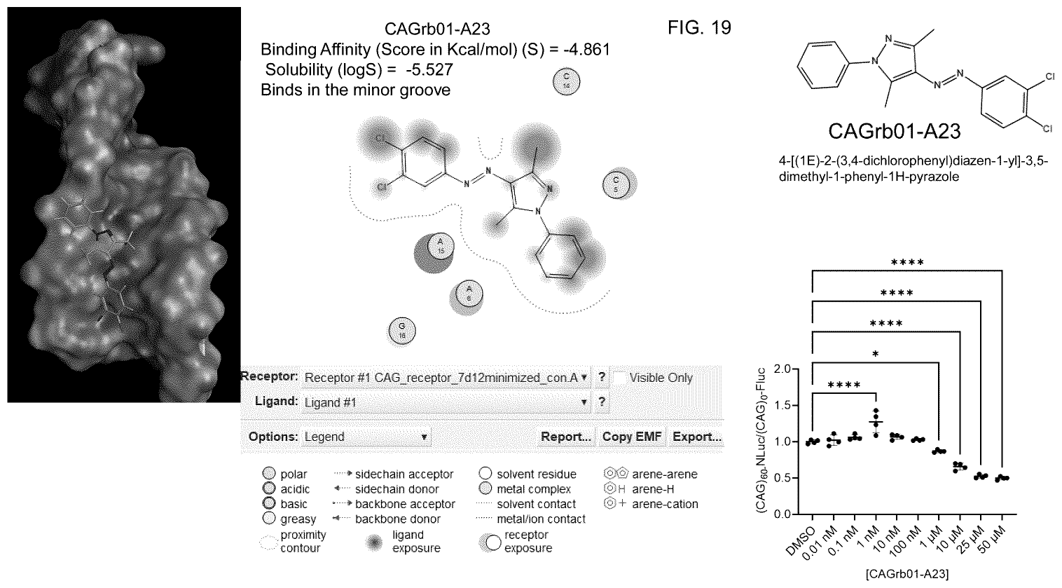

The identified differential gene expression changes occurred in pathways reported to be affected in CAG SCA mouse models such as ion transmembrane transport, neuron projection morphogenesis and neuroactive ligand-receptor interaction (Fl. 1G). Multiple genes previously reported to show differential gene expression in a variety of CAG SCA mouse model cerebellum were identified as being differentially expressed in both clone 15 and 37 (FIG. 26C). Several of these genes showed changes in transcript expression in the same direction as reported in CAG SCA mouse models and were validated via qPCR. These included GRID2 and GABRA3 which encode ion channel subunits, and CLMN and ANK2 which encode proteins that are associated with the actin cytoskeleton (FIG. 1H, FIG. 11, and FIG. 26D-26F).

Together, these data demonstrate that CAG expansion containing clonal cell lines display phenotypic and molecular hallmarks consistent with mouse models of CAG SCAs. To enable small molecule screening in this system, we sought to identify a suitable positive control from the literature. Because no compounds have been published to selectively reduce CAG expansion transcript levels, we utilized a CAG targeting siRNA (si(CAG)7], based on the CAG targeting ASO studied in the context of SCA1 and SCA313, as a positive control with a scrambled siRNA (siCti) as a negative control. Clone 15 showed a 65.4% selective reduction in (CAG)60 relative to (CAG)D and a 46.4% reduction in polyQ-NLuc relative to FLuc (both P<0.0001) on si(CAG)7 treatment compared to scrambled siRNA. For clone 37, there was a 57.0% selective reduction of (CAG)60 and a 38.1% selective reduction of polyQ-NLuc (both P<0.0001) relative to the no-repeat controls for si(CAG) treatment compared to scrambled siRNA (FIG. J and FIG. 1K). The Z′ scores between 0.5 and 1 (FIG. 1i and FIG. 1K) indicate that this assay is suitable for screening at the protein and RNA levels in clone 37 and at the protein level in clone 15.

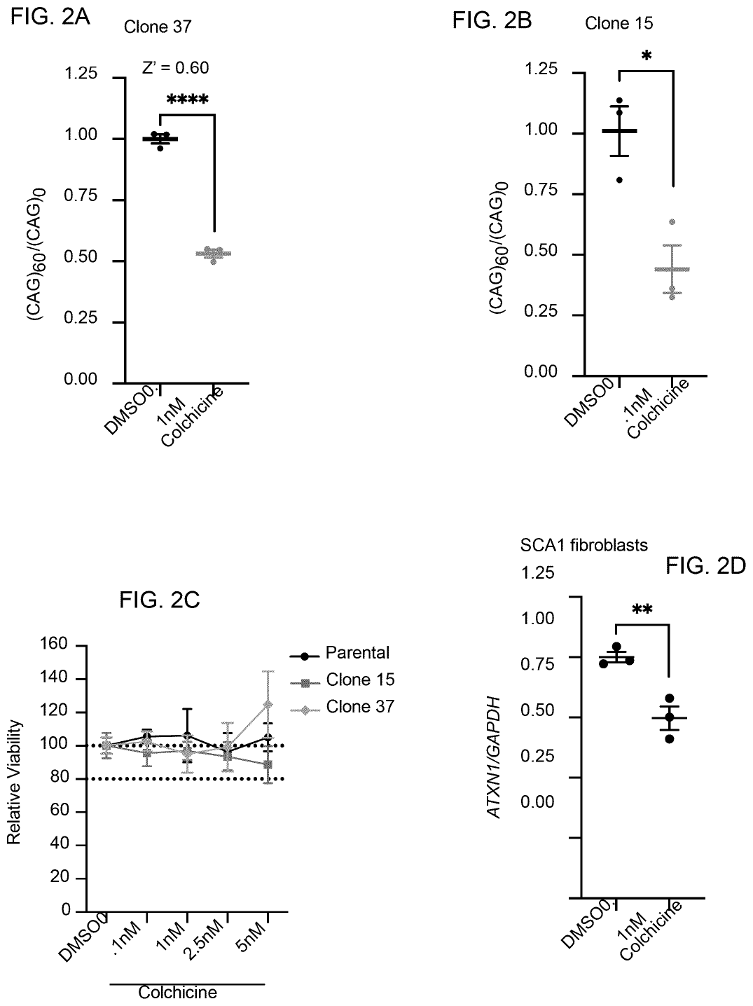

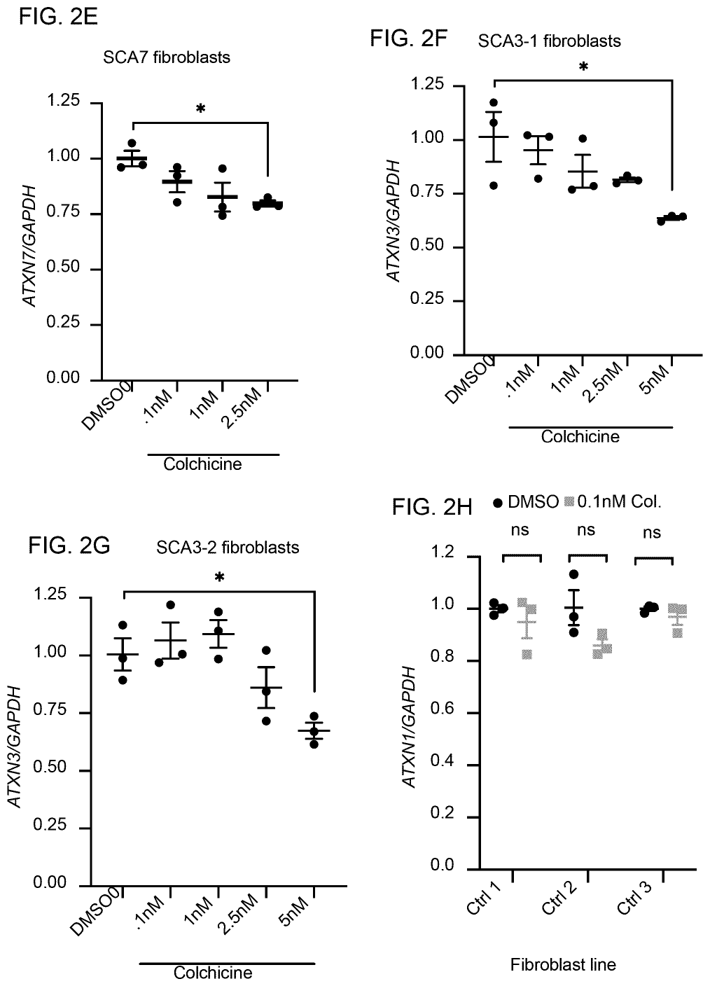

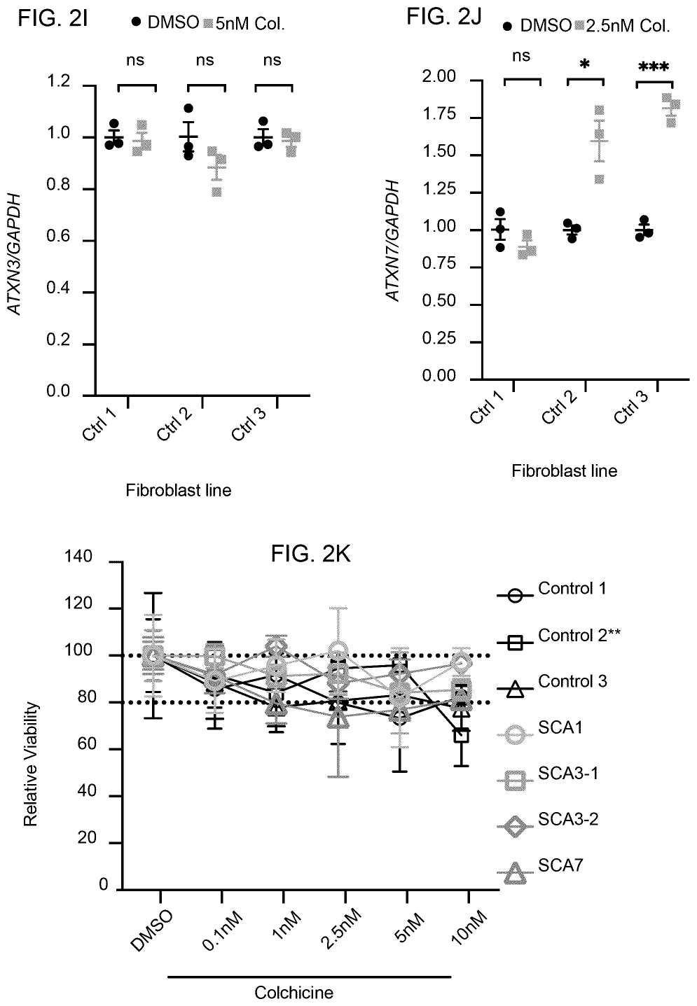

The FDA approved compound Colchicine regulates CAG expansion RNA levels: To ensure we had an appropriate comparison for screening a small molecule library prepared in DMSO, we sought a small molecule positive control that could regulate expression of CAG expansion transcripts. Based on its ability to regulate expression of CUG expansion RNAs, we tested and identified the microtubule inhibitor colchicine as a DMSO-soluble small molecule regulator of CAG expansion transcript abundance (FIG. 2). In clone 37, a 24 hour treatment of 0.1 nM colchicine resulted in a 46,9% selective reduction (P<0.0001) of (CAG)60 levels compared to DMSO treatment, with a Z′ score of 0.6 (FIG. 2A) indicating an assay suitable for small molecule screening. In clone 15, 0.1 nM colchicine treatment for 48 hours resulted in a selective reduction of CAG expansion transcript levels by 57.1% (P=0.0159; IG. 2B). In both clonal cell lines and parental HEK293T cells, colchicine did not induce a reduction of cell viability within and beyond the effective dose range (FIG. 2C).

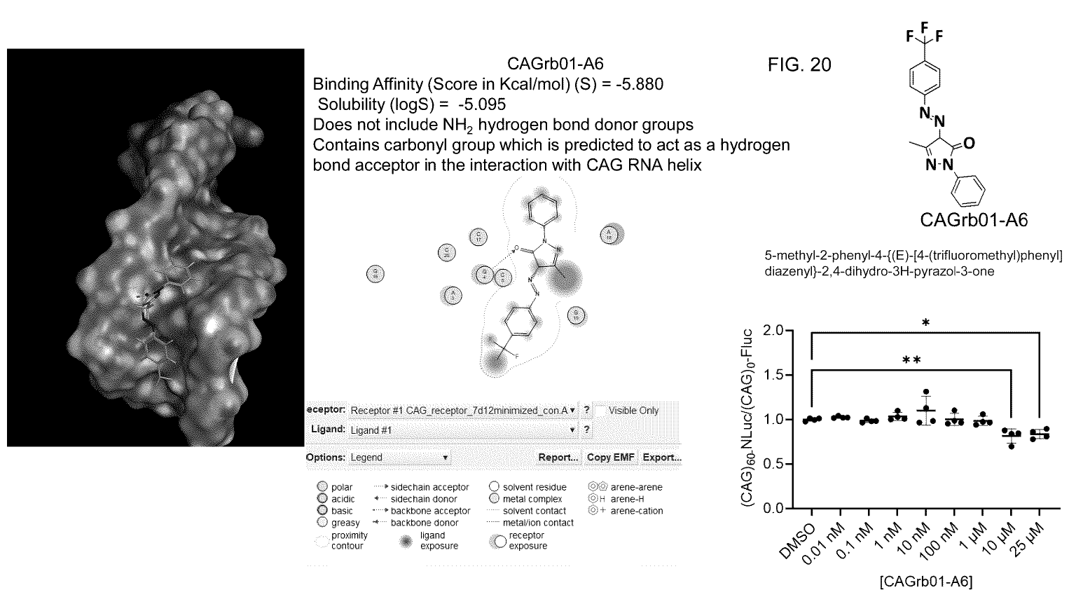

To further validate the use of colchicine as a positive control small molecule in CAG SCAs, we treated SCA1, SCA3 and SCA7 patient derived fibroblast lines with colchicine for 48 hours. In an SCA1 patient derived cell line, 0.1 nM colchicine reduced expression of ATXN1 by 25.3% (P=0.009; FIG. 20). In SCA3 and SCA7 no effect was seen at 0.1 nM for 48 hours so a wider dose range was tested (FIG. 2E-FIG. 2G). In an SCA7 patient-derived fibroblast line, 2.5 nM colchicine led to a 20.2% reduction in ATXN7 expression levels (P=0.049; FIG. 2E).

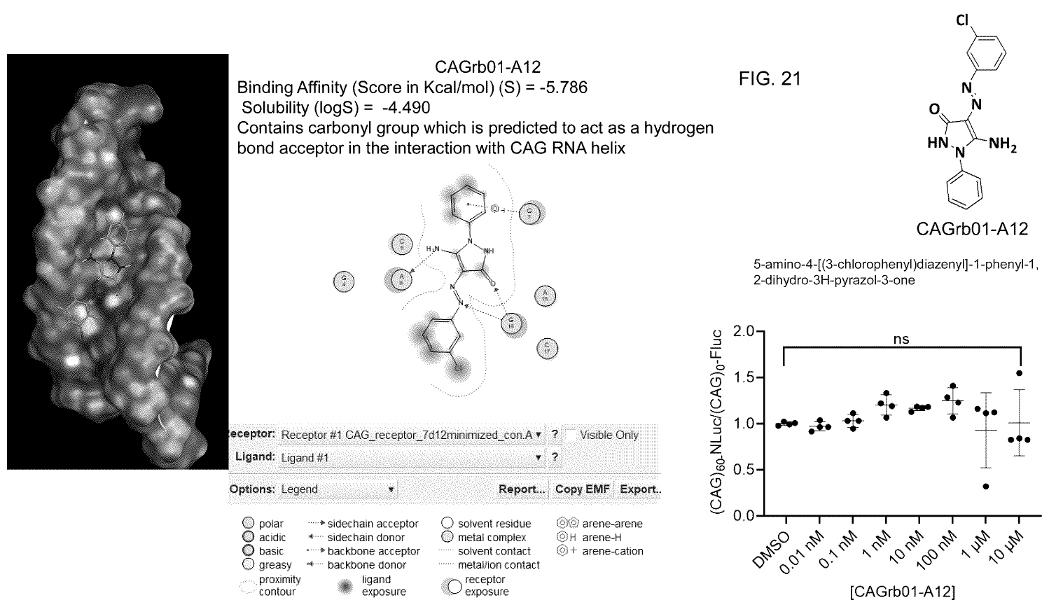

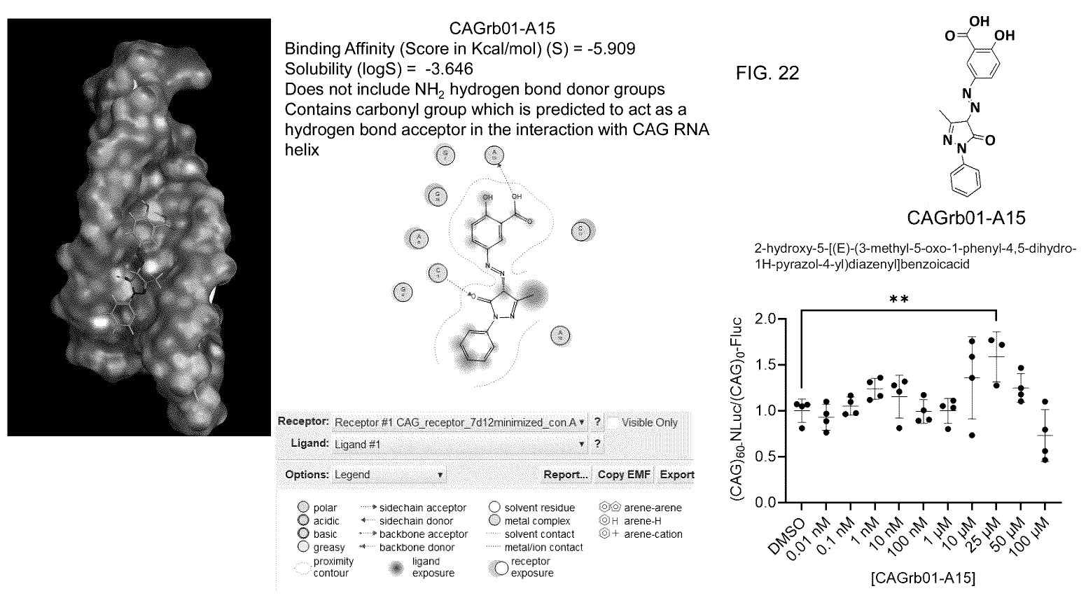

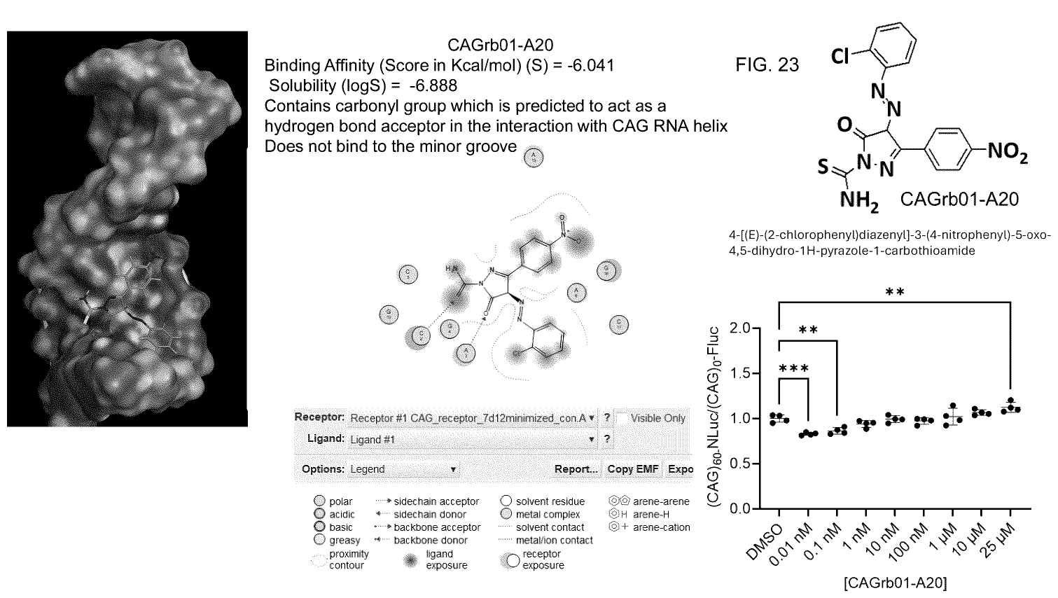

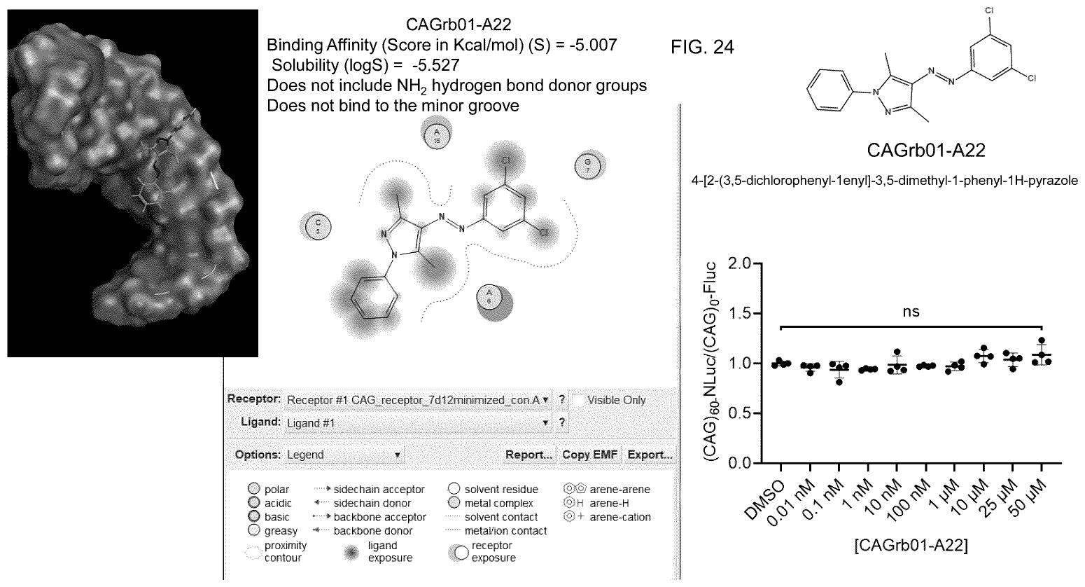

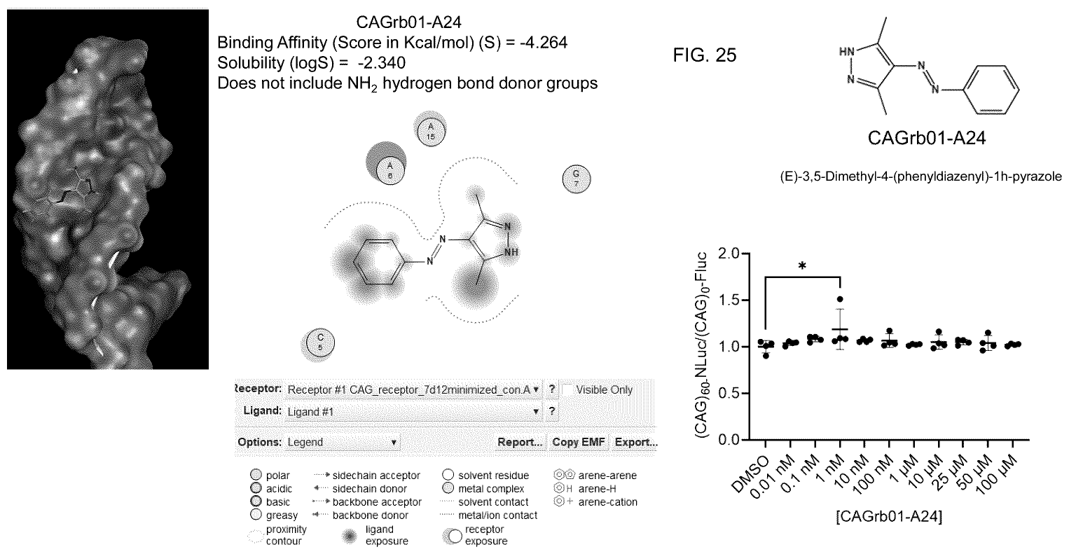

Two different SCA3 patient derived cell lines showed a reduction of ATXN3 expression following treatment with 5 nM colchicine: in the SCA3-1 line, this was a reduction by 37.7% (P=0.021; FIG. 2F), and in SCA3-2 a reduction of ATXN3 expression by 33.1% (P=0.042; FIG. 2G). At these doses where a reduction in expression was seen in patient derived fibroblast lines, there was, however, no effect in three control fibroblast lines of 0.1 nM colchicine on ATXN1 expression (FIG. 2H) or of 5 nM colchicine on ATXN3 expression (FIG. 21) and two out of three control fibroblast lines showed a significant increase in expression of ATXN7 following treatment with 2.5 nM colchicine (Control 2: 59.5% increase, P=0.013; Control 3: 81.5% increase, P=0.0002; FIG. 2J).

These data demonstrate that colchicine regulates CAG transcript levels in a repeat length dependent manner and suggests differences in the regulation of control allele transcript expression for ATXN7 versus ATXN1 and ATXN3. Although colchicine treatment was well tolerated in clone 15 and 37 (FIG. 2C), an overall trend of reduced cell viability was seen for colchicine treatment in fibroblasts but only control 2 showed a significant reduction in cell viability at the highest dose tested (10 nM, 33.9% reduction, P=0.002; FIG. 2K).

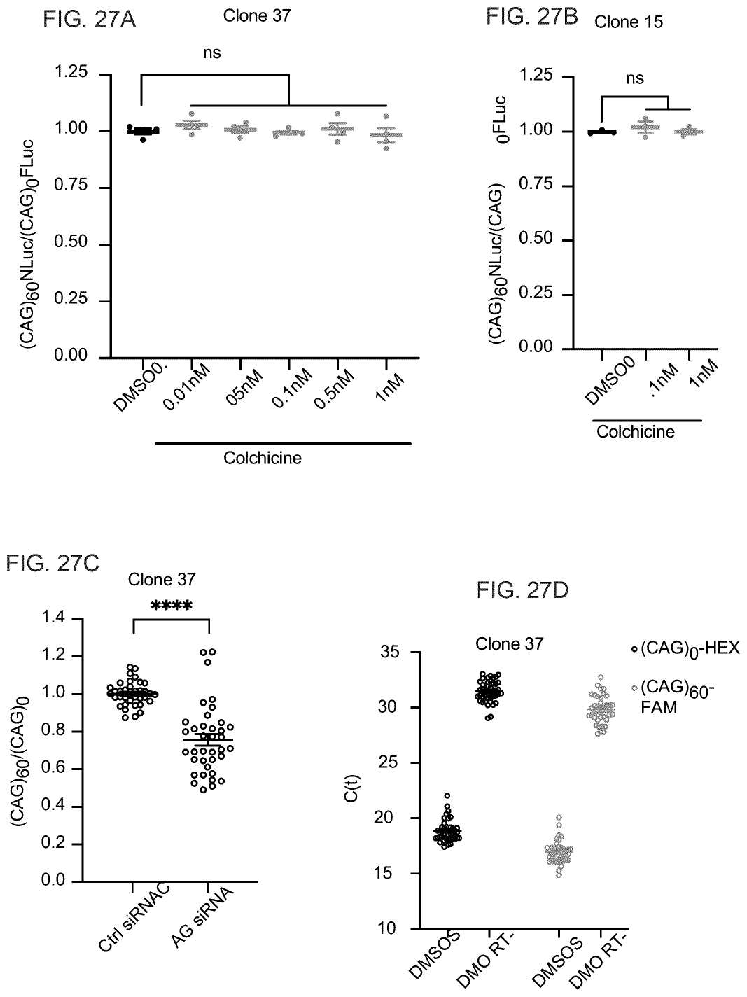

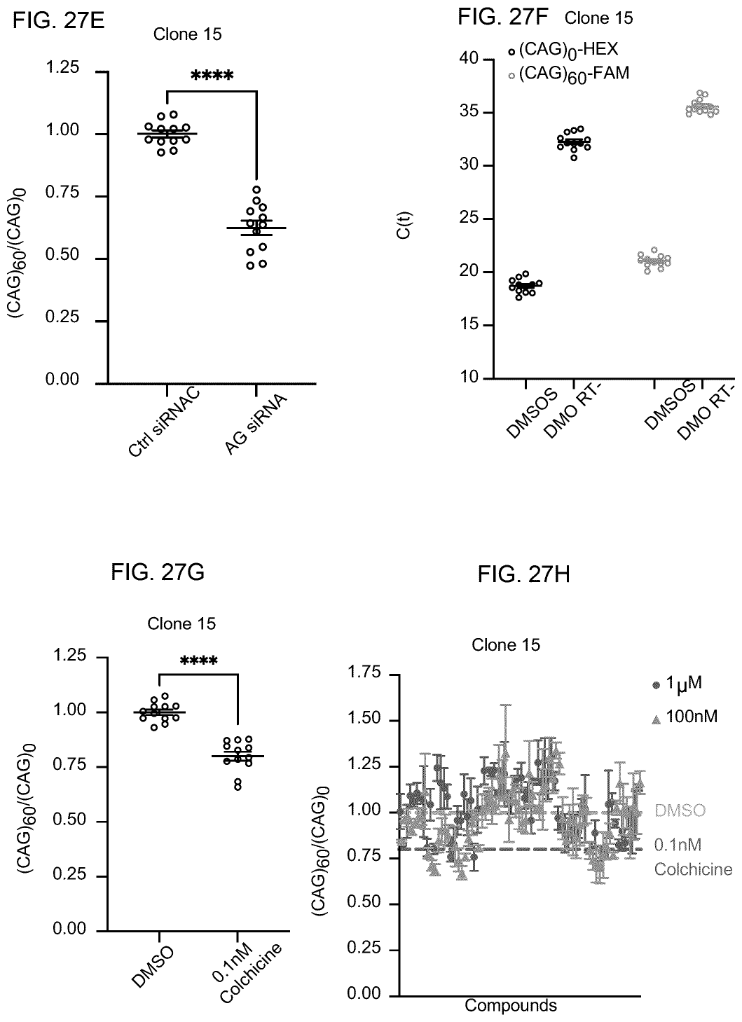

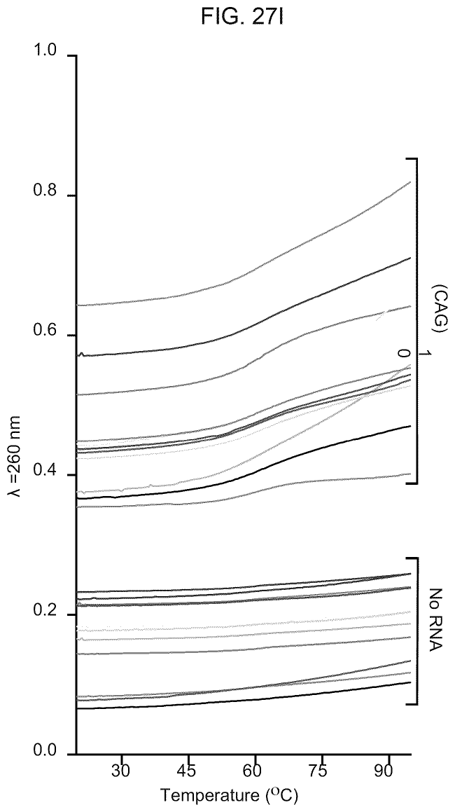

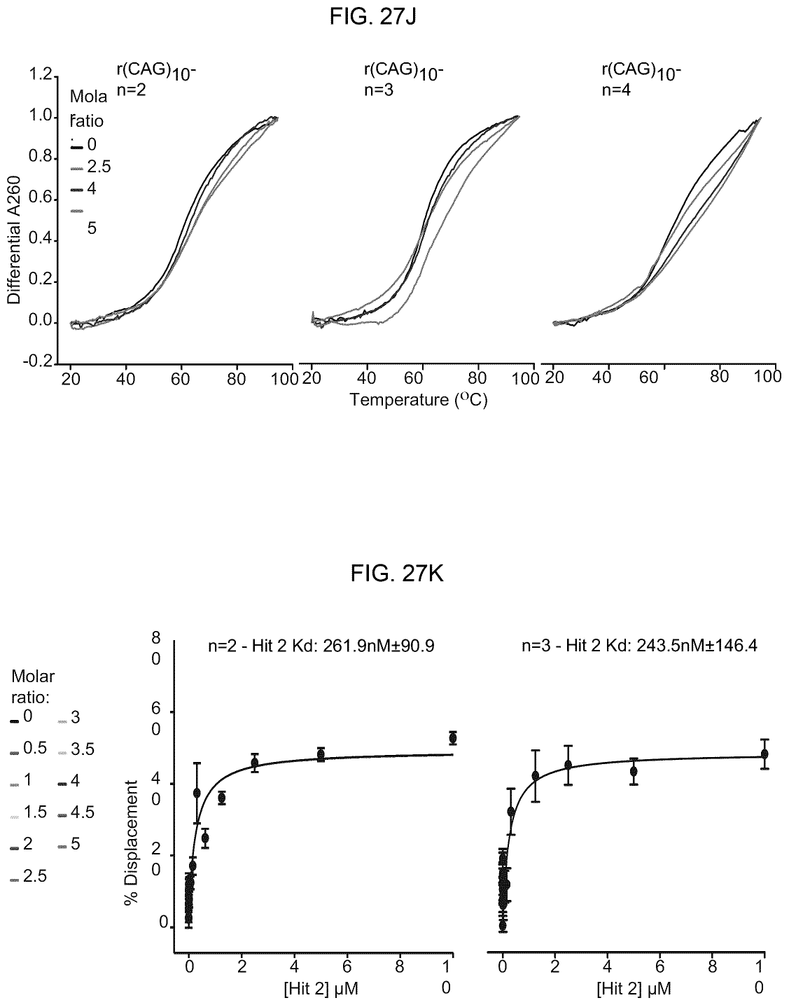

Finally, despite promising data of colchicine on RNA expression, in both clone 15 and clone 37, colchicine treatment failed to selectively reduce poly-NLuc expression levels (FIGS. 27A, 278), perhaps suggesting that sufficient expansion RNA is still being transcribed and exported to the cytoplasm to maintain consistent levels of polyQ proteins. Together, these data provide proof-of-concept that small molecules can regulate CAG expansion transcripts in a repeat length dependent manner across multiple CAG expansion diseases.

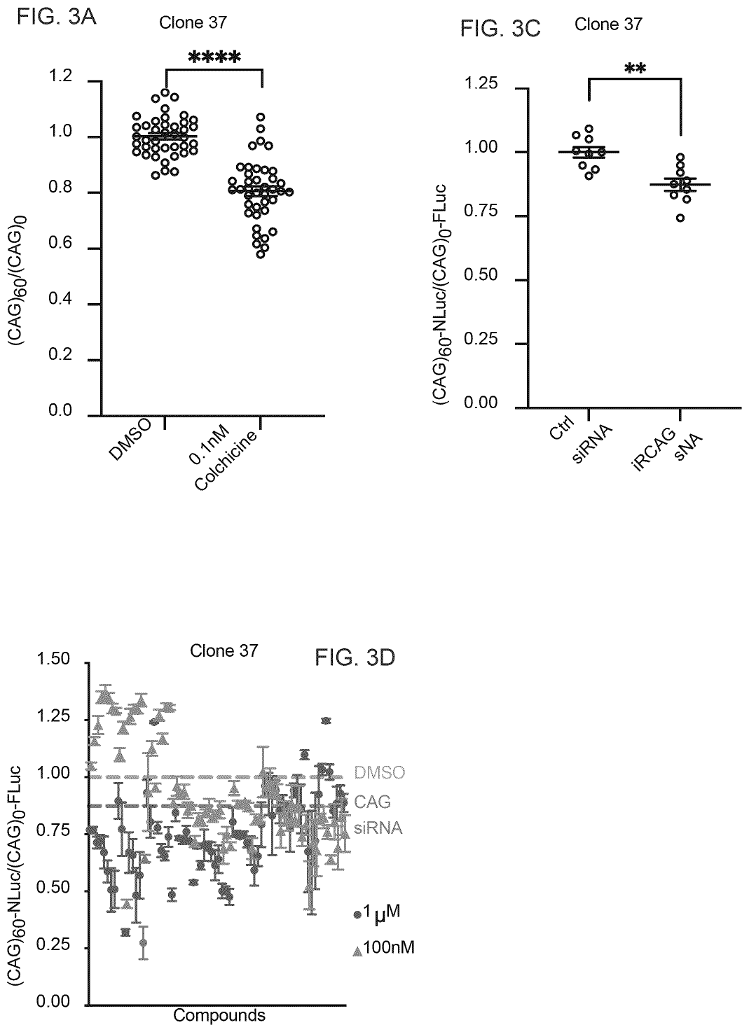

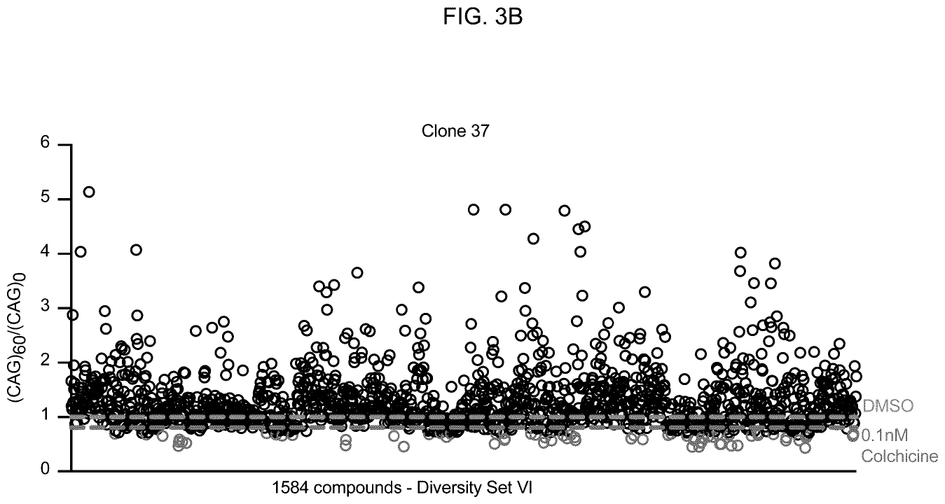

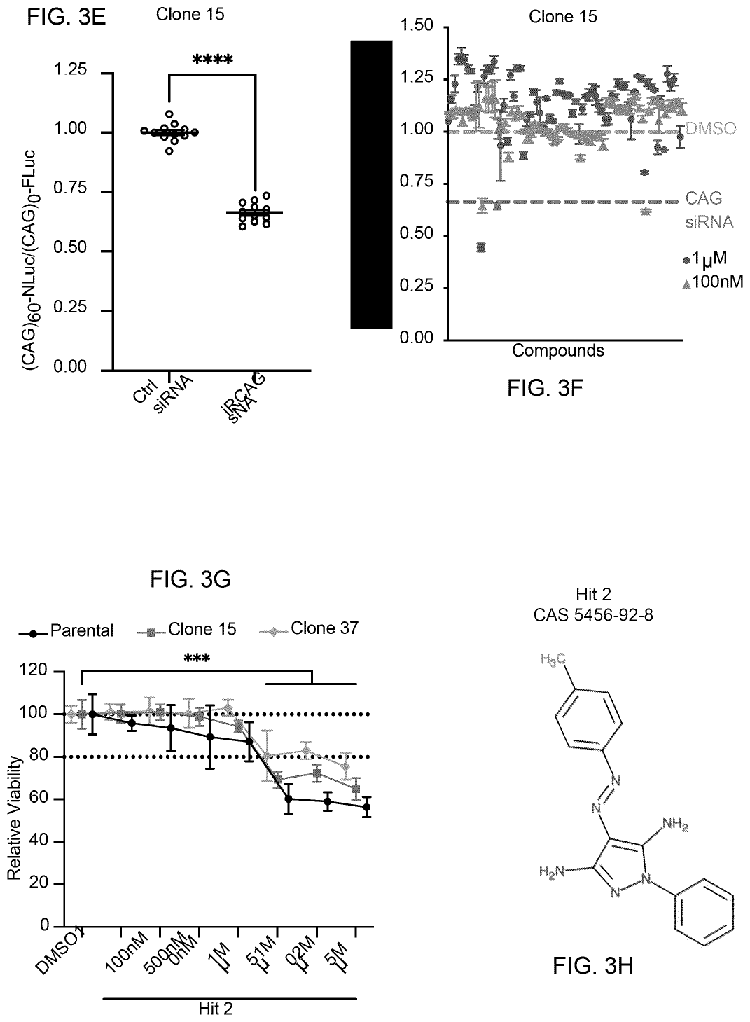

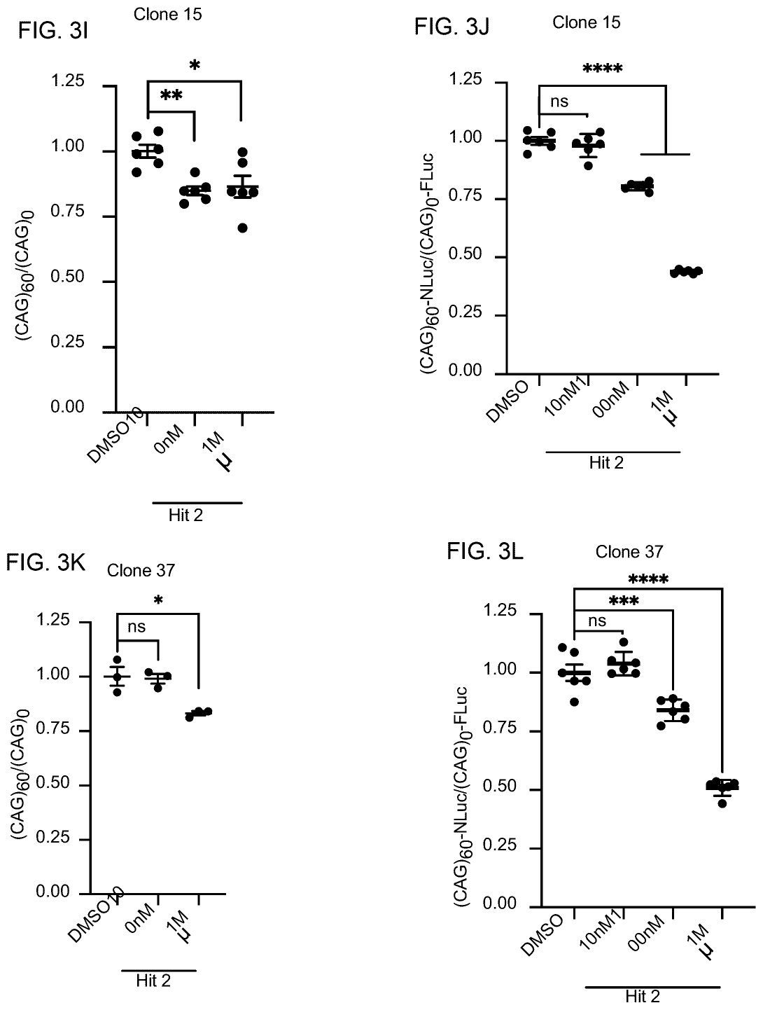

A CAG repeat selective screen identifies a pyrazole-based compound that regulates CAG expansion RNA levels. To maximize the potential of identifying different structural classes of compounds with the potential to regulate CAG transcript abundance through different mechanisms, we performed a screen of 1584 structurally diverse compounds from the National Cancer Institute Developmental Therapeutics Program (NCI DTP) Diversity Set VI. Compounds were screened at 1 μM in 0.01% DMSO in clone 37 for 24 hours, utilizing both colchicine and si(CAG)7 as positive controls and scrambled siRNA and DMSO as negative controls (FIG. 3). Across the screen, 0.1 nM colchicine showed a 19.6% reduction in (CAG)60 relative to (CAG)0 (P<0.0001; FIG. 3A) and CAG targeting siRNA selectively reduced (CAG)60 abundance by 24.5% (P<0.0001; FIG. 27C). Control reverse transcriptase reactions were used to confirm that the RT-qPCR screening assay behaved as expected (FIG. 27D). The screen of 1584 Diversity set VI compounds identified 72 compounds that selectively reduced (CAG)60 expression beyond the 90% confidence interval (CI) of the mean for both positive controls (lower 90% C1 of mean: 0.1 nM colchicine=77.6%, si(CAG)7=70.6%) reflecting a hit rate of 4.54% (FIG. 38). To ensure compounds identified in this initial RNA expression screen (FIG. 38) also selectively reduced poly protein levels and were acting independent of construct integration sites, the 72 compounds were rescreened at 1 μM and 100 nM in both clone 15 and clone 37 for selective reduction of polyQ levels and in clone 15 for selective reduction of CAG RNA levels. For clone 37, the CAG targeting siRNA selectively reduced polyQ-NLuc expression by 12.6% (P=0.0011; FIG. 3C) with 65 out of 72 compounds selectively reducing polyQ-NLuc expression below this threshold for at least one dose (FIG. 3D). In clone 15, si(CAG)7 selectively reduced expression of (CAG)60 by 37.6% (P<0.0001, FIG. 27E) and as with the RNA expression screen in clone 37, control reverse transcriptase reactions were used to confirm that the RT-qPCR screening assay behaved as expected in clone 15 (FIG. 27F). Colchicine selectively reduced (CAG)60 by 20.1% (P<0.0001, FIG. 27G) in clone 15 with 15 of 72 compounds selectively reducing (CAG)60 expression below this threshold for at least one dose (FIG. 27H). In clone 15, si(CAG)7 selectively reduced expression of polyQ-NLuc by 33.6% (P<0.0001; FIG. 3E) with three compounds (Hits 1, 2 and 3) selectively reducing expression of polyQ-NLuc below this threshold (FIG. 3F); all three compounds also passed the threshold for reduction of (CAG)60 expression in clone 15 (FIG. 27H). Of these three Hit compounds, Hit 2 showed the greatest selective reduction of poly-NLuc expression in clone 37 (72.6%; FIG. 3D) and was the third best performing compound in the clone 37 RNA expression screen (selective reduction of (CAG)60 by 54.5%; FIG. 3B) out performing Hit 1 and 3 by >20%.

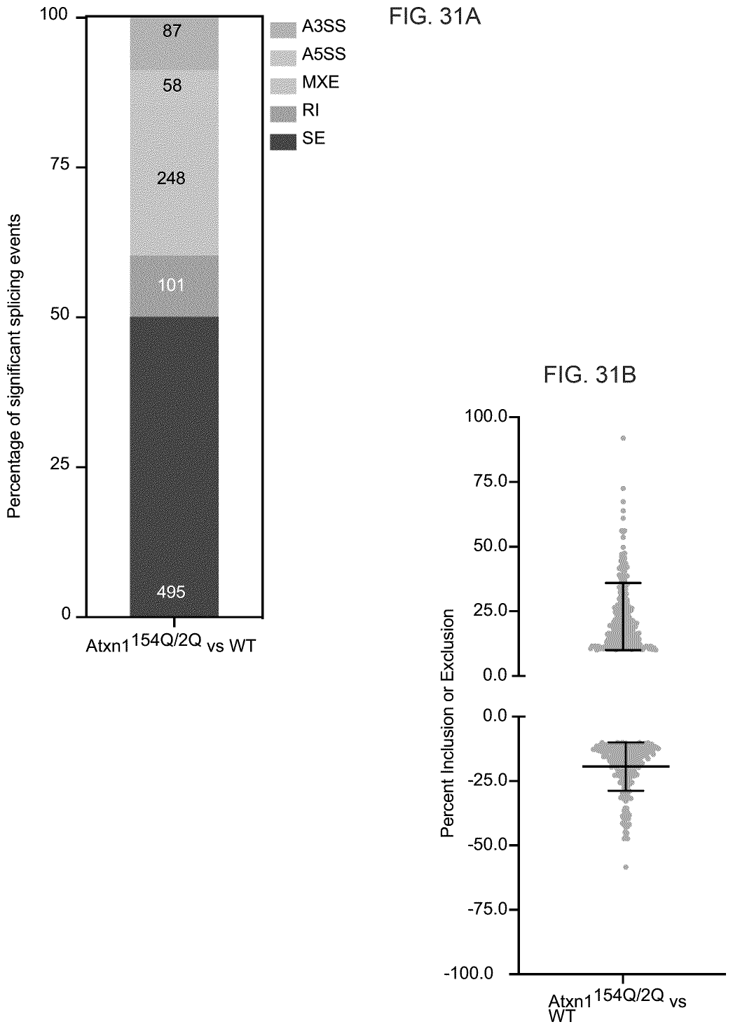

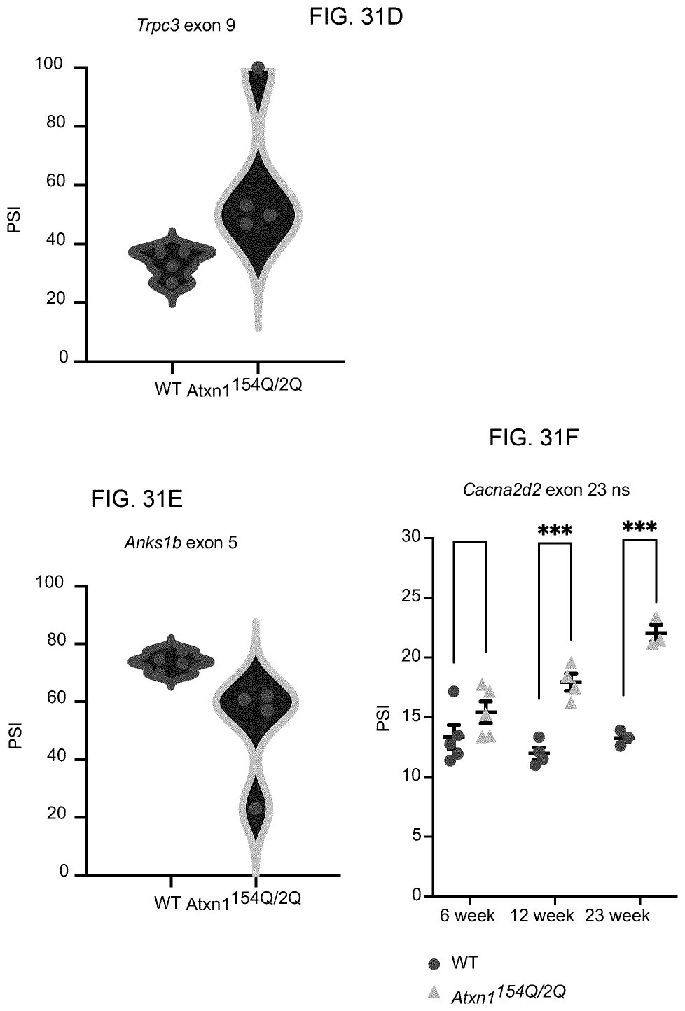

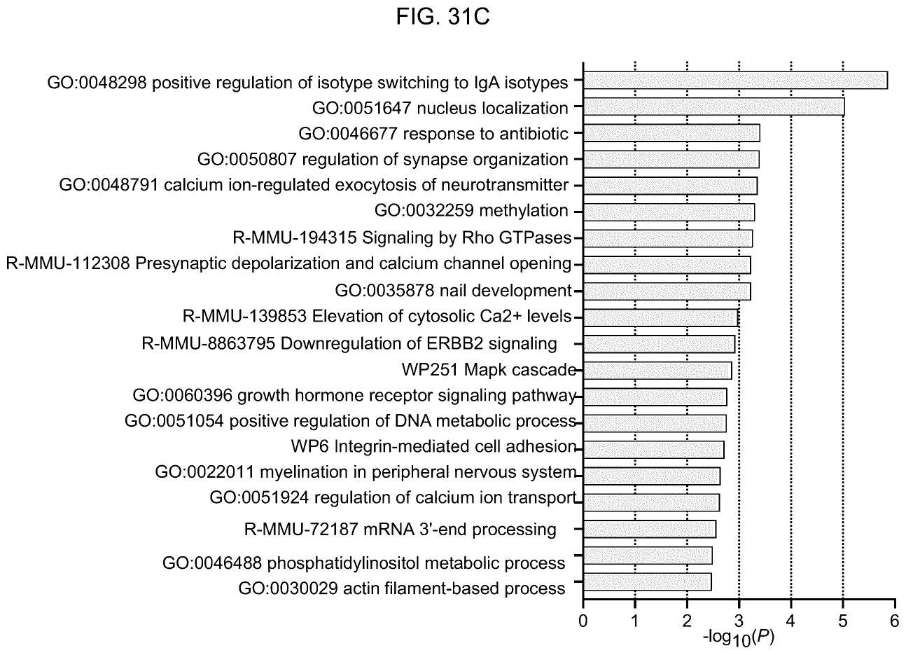

Therefore, Hit 2 was selected for further investigation as a regulator of CAG expansion expression. Hit 2 (IUPAC name: 4-[(4-methylphenyl)diazenyl]-1-phenylpyrazole-3,5-diamine; CAS number: 5456-92-6) is a pyrazole based compound (FIG. 3G) soluble in DMSO and methanol, but not in PBS or water. Hit 2 has limited published data for cell-based assays and so we initially set out to identify a dose range that did not negatively impact cell viability in HEK293T cells. In parental HEK293T cells and clones 15 and 37, Hit 2 was well tolerated up to 1 μM but at 5 μM, 10 μM and 25 μM, Hit 2 significantly reduced cell viability across all three lines. In clone 37 there was a 17% or greater reduction in cell viability at doses greater than 1 μM, for clone 15 this was a>27% reduction in viability and >39% reduction in viability for parental HEK293T cells compared to DMSO treatment for each cell line (P>0,001; FIG. 3H). Beyond 25 μM, Hit 2 crashed out of solution in cell culture media and formed crystals. Within the tolerated dose range, clone 15 showed a 15.2% (P=0.0066) and a 13.6% (P=0.0142) selective reduction in (CAG)60 expression following 24 hour treatment with 100 nM and 1 μM Hit 2, respectively (FIG. 31). This corresponded to a 19.5% and 56.2% selective reduction (both P<0.0001) in polyQ-NLuc expression at 100 nM and 1 μM of Hit 2, respectively (FIG. 3J). Similarly, in done 37, at 100 nM, while Hit 2 did not significantly reduce expression of (CAG)60, there was a 16.0% reduction in polyQ-NLuc (P=0.0006) and at 1 μM there was a 17% selective reduction (P=0.0136) in (CAG)60 and a 49.1% reduction in polyQ-NLuc (P<0.0001; FIG. 3K, and FIG. 3L). Together these data demonstrate that Hit 2 is a novel compound capable of regulating expression of CAG expansion transcripts and polyQ proteins.

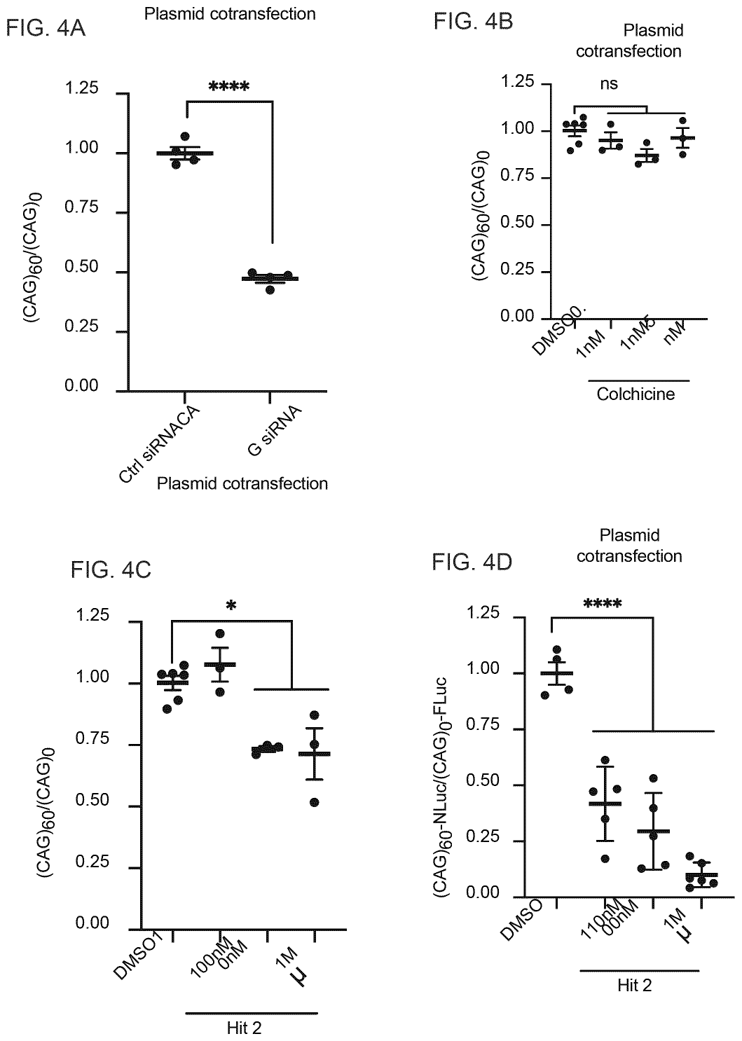

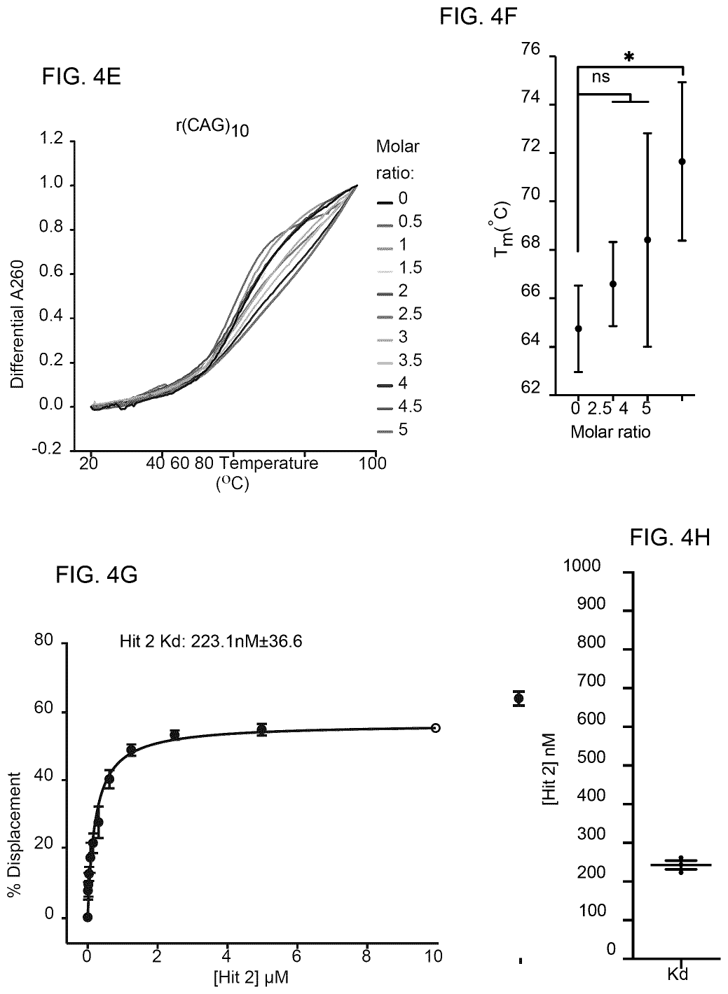

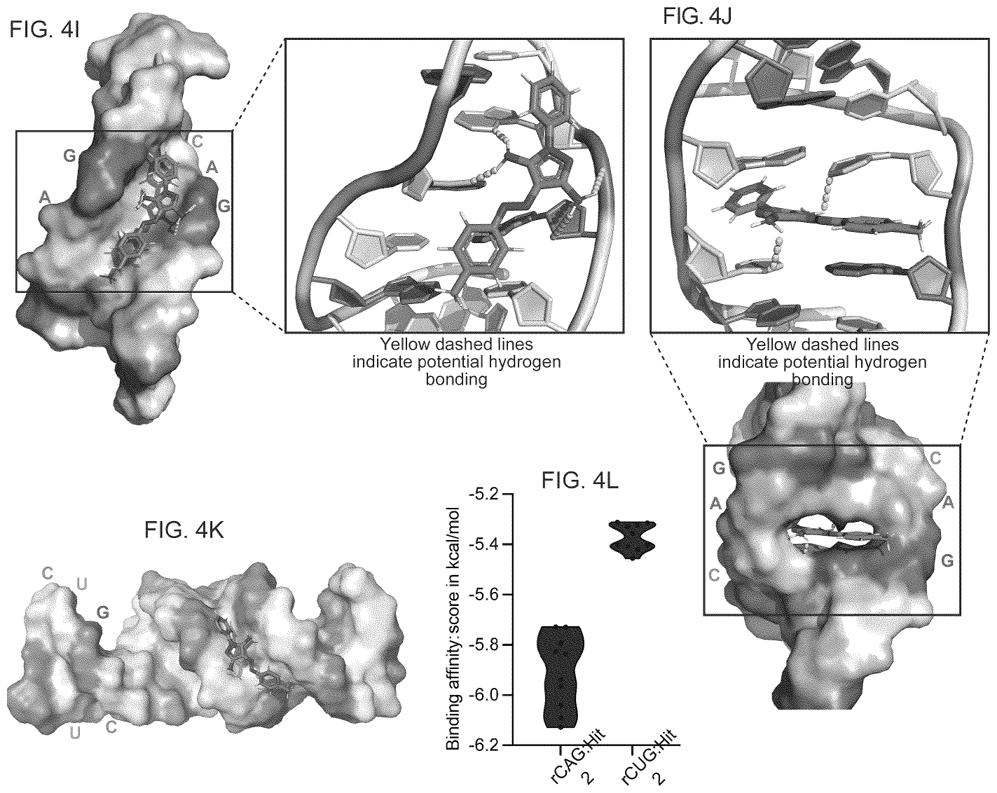

Colchicine and lead candidate compound Hit 2 regulate CAG expansion RNA abundance through distinct mechanisms: We previously demonstrated that the mechanism of action of the microtubule inhibitor colchicine in DM1 is dependent on CUG repeats being integrated into the cell genome rather than being expressed ectopically via plasmid overexpression. To test if this was consistent for the mechanism of action of colchicine on CAG RNA expression, we performed transient co-transfections of the ATG-(CAG)60-Myc-NLuc and ATG-(CAG)0-Myc-FLuc constructs used to generate clone 15 and 37 screening cell lines. Following transient co-transfection, our CAG targeting siRNA positive control selectively reduced (CAG)60 expression relative to (CAG) expression by 52.8% (P<0.0001; FIG. 4A) consistent with the effect seen in done 15 and clone 37 (FIG. 1J). Treatment with colchicine at 0.1 nM, nM and 5 nM did not affect expression of (CAG) in the co-transfection system (FIG. 48). These data are consistent with the mechanism of action through which cochicine regulates expression of CUG expansion RNAs22. In contrast to colchicine, treatment of the co-transfection system with Hit 2 at 100 nM and 1 μM reduced (CAG) expression relative to (CAG)0 expression by 26.8% (P=0.0175) and 28.8% (P=0.0110), respectively (FIG. 4C). This plateauing effect of Hit 2 treatment on (CAG)60 expression is consistent with the effect of Hit 2 in clone 15 (FIG. 31). Similar to the dose dependent effect of Hit 2 on polyQ-NLuc expression in both clone 15 and clone 37, with a greater effect on protein levels than RNA levels (FIG. 3J, and FIG. 3L), in the co-transfection system Hit 2 selectively reduced polyQ-NLuc expression by 58.1% at 10 nM. 70.5% at 100 nM, and 90% at 1 μM (all P<0.0001; FIG. 4D). Together these data demonstrate that Hit 2 and colchicine regulate expression of CAG expansion RNAs through distinct mechanisms.

To understand more about the potential mechanism of action of Hit 2, we performed biophysical and molecular docking studies to investigate if Hit 2 has the potential to bind expansion RNAs. UV melting assays were performed to determine the thermal stability of the target RNA in the presence of increasing concentrations of Hit 2. UV melting of r(CAG)10 with Hit 2 at increasing molar ratios demonstrated a stabilising effect of Hit 2 on (CAG)10 RNA structure with a shift in the melting curve to higher temperatures (FIGS. 4E, 271, 4J) corresponding to a Tm of 6.9 C at a molar ratio of 5 (P=−0.0312, FIG. 4F). To characterise the affinity of Hit 2 for CAG RNAs, we performed a fluorescent indicator displacement assay (FID) with r(CAG)10 and SYBR Safe as the fluorescence indicator. Our results demonstrated that Hit 2 effectively displaced approximately 50% of SYBR Safe at a concentration of 2 μM (FIG. 4G, 27K), with an average dissociation constant (Kd) of 242.8 nM±11.21 (FIG. 4H; Exp1: Kd=223.1nMt36.6, P=0.0001, FIG. 4G; Exp2: Kd=261.9 nM90.9, P=0.0108, FIG. 27K; Exp3: Kd=243.5 nM±146.4, P=0.0001, FIG. 27K). Together these data suggest that the presence of Hit 2 increases the thermal stability of CAG repeat RNAs and that Hit 2 is capable of directly interacting with CAG repeat RNA in the concentration range where activity is seen in cell culture. To investigate the possible interaction between Hit 2 and CAG repeat RNAs, in silico molecular modelling and small molecule docking was utilized to identify potential binding modes. Docking identified multiple possible binding modes for the interaction of Hit 2 with CAG repeat RNA (FIG. 4I, and FIG. 4J). The groove binding mode allows for hydrogen bonds to form between one of the amine groups on Hit 2 and the AA stacked mismatch in the CAG RNA helix and the other amine group forms hydrogen bonds with the backbone of the neighbouring G (FIG. 4I). Interestingly, molecular docking predicts that Hit 2 also prefers the minor groove when interacting with a CUG RNA duplex (FIG. 4K), however, this is a less favorable interaction with a weaker binding affinity (−5.38 kcal/mol) than for Hit 2 binding to CAG repeat RNA minor groove (−5.89 kcal/mol; FIG. 4L). As a potential intercalator, docking predicts the interaction is driven primarily by the terminal benzene rings of Hit 2 which establish non-polar contacts with the AA mismatch and the GC pair, while fewer hydrogen bonds are predicted between the amine groups and the participating nucleotides at the intercalation site (FIG. 4J). Together, this strengthens the potential for interaction between Hit 2 and CAG repeat RNAs suggesting an interaction possibly through Hit 2 adopting a conformation that is suitable for binding to the minor groove of CAG repeat RNA where the interaction is stabilized by hydrogen bonds between Hit 2 and the AA stack and backbone, although other binding modes, including intercalation, are also possible. Additionally, these studies predict that Hit 2 binds less efficiently to CUG repeat RNAs than to CAG repeat RNAs, consistent with cell studies.

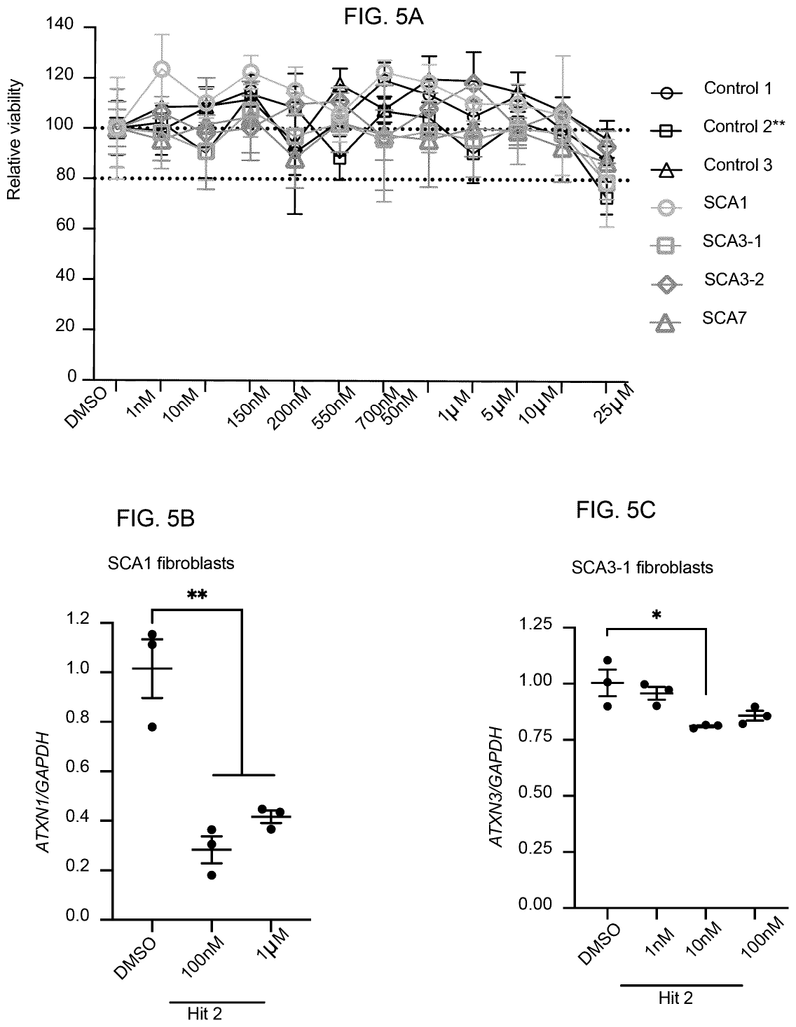

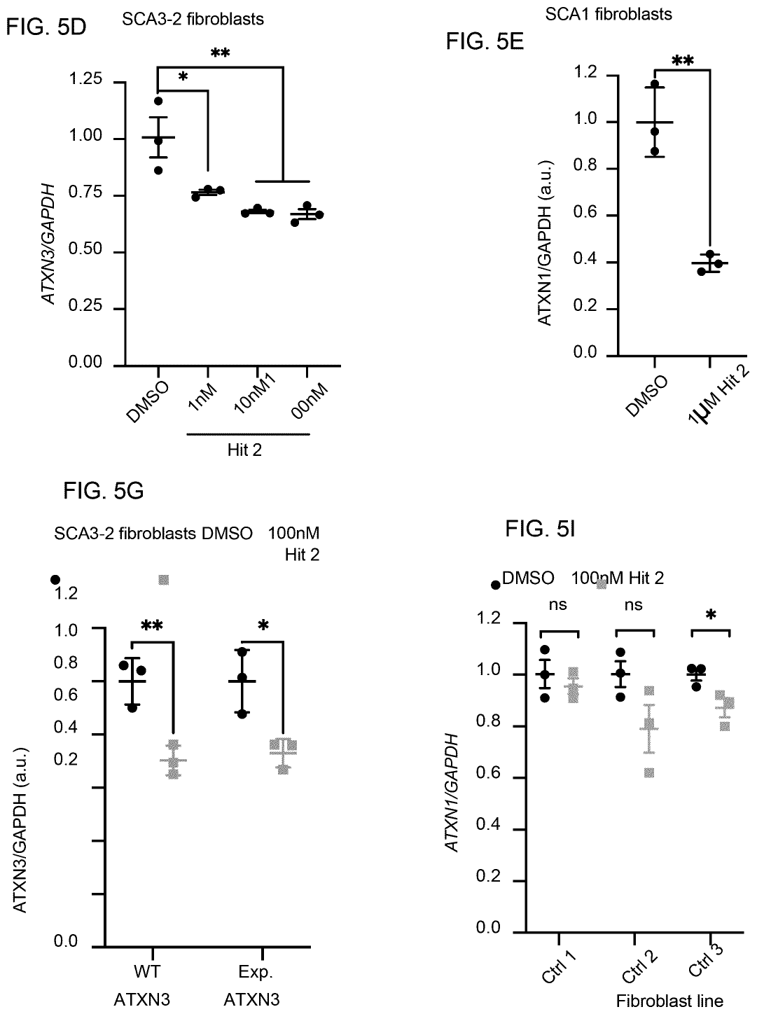

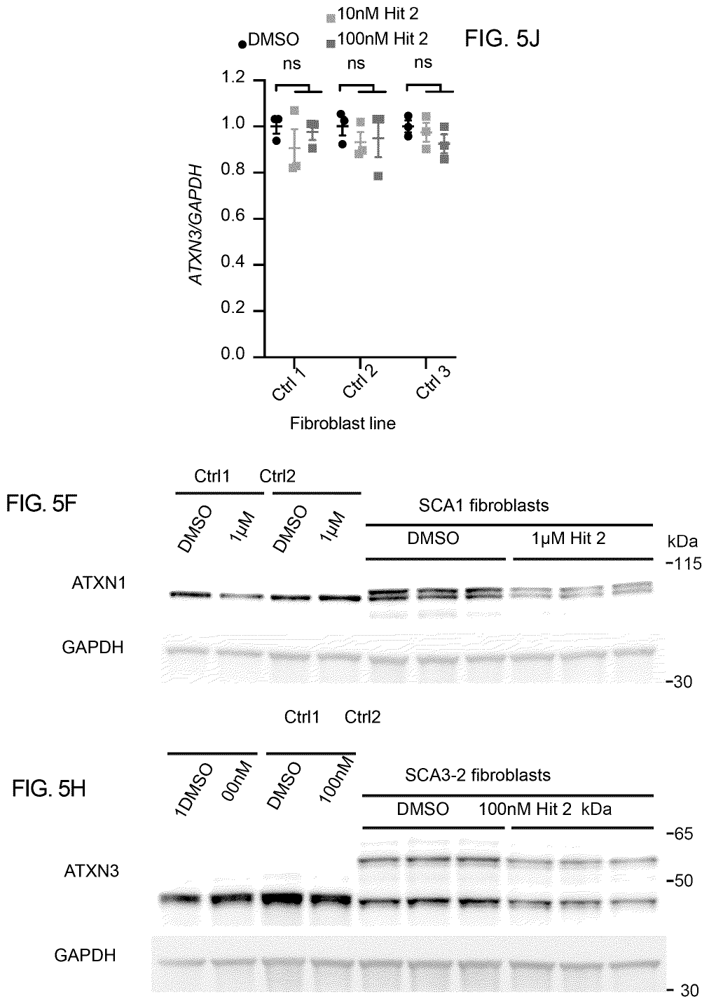

Lead candidate compound Hit 2 reduces expression of CAG expansion transcripts across multiple patient derived fibroblast lines: To investigate the potential of Hit 2 as a regulator of CAG expansion transcripts in the context of SCA disease causing genes, we treated SCA1, SCA3 and SCA7 patient derived fibroblast lines with Hit 2 for 48 hours. Again, we initially set out to identify a dose range that did not negatively impact cell viability. In three control lines, one SCA1, two SCA3 and one SCA7 fibroblast line, Hit 2 was well tolerated up to 10 μM. At 25 μM, there was an overall trend of reduced cell viability with Hit 2 significantly reducing viability by 27.1% for Control 2 (P=0.0047) compared to DMSO treatment (FIG. 5A).

In the SCA1 patient derived cell line, 100 nM Hit 2 reduced expression of ATXN1 by 73,2% (P=0,0013) and at 1 μM Hit 2, ATXN1 expression was reduced by 59.9% (P=0.0036; FIG. 5B). For SCA3-1, a 19.3% reduction in ATXN3 expression was seen at 10 nM (P=0.0186. FIG. 5C) and for SCA3-2, at 1 nM, 10 nM and 100 nM Hit 2 reduced ATXN3 expression by 24.2% (P=0.0246), 32.7% (P=0.0046) and 33.9% (P=0.0037), respectively (FIG. 5D). At the protein level, we detected a 60.3% reduction in ATXN1 expression at 1 μM Hit 2 in SCA1fibroblasts (FIG. 5E, and FIG. 5F; P=0.0024) and a 29.8% (P=0.0076) and 24.1% (P=0.0221) reduction in normal and expanded ATXN3 protein levels in SCA3 fibroblasts (FIG. 5G, and FIG. 5H); effect sizes consistent with the effect seen on RNA expression levels. These data demonstrate that Hit 2 can reduce ATXN protein and RNA levels across patient derived models from multiple CAG expansion spinocerebellar ataxias.

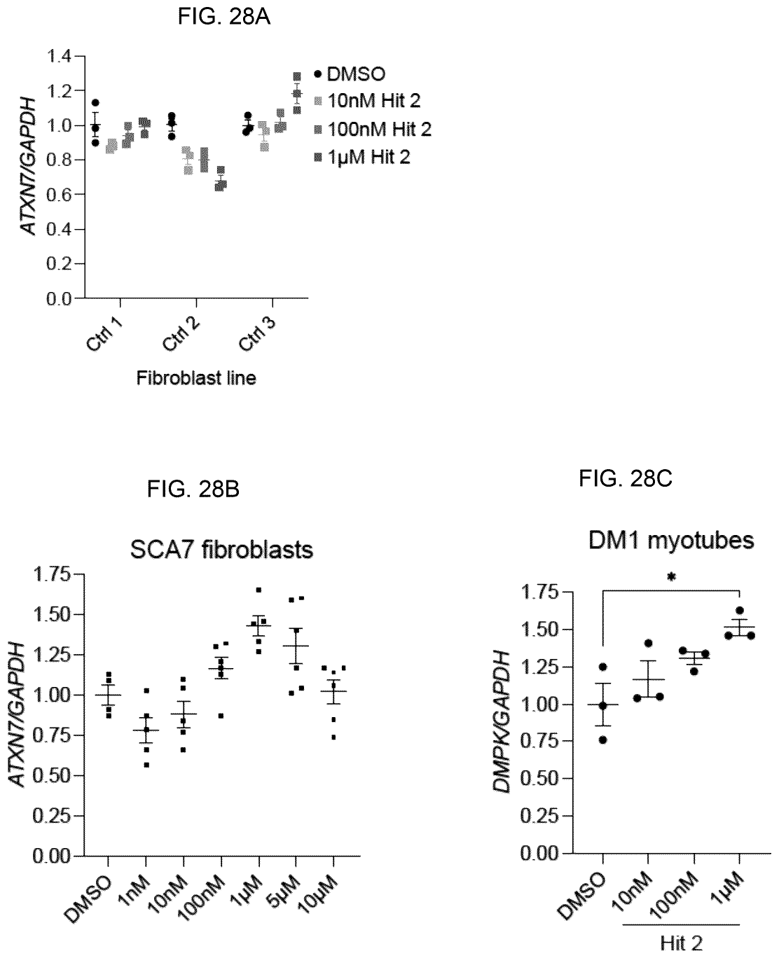

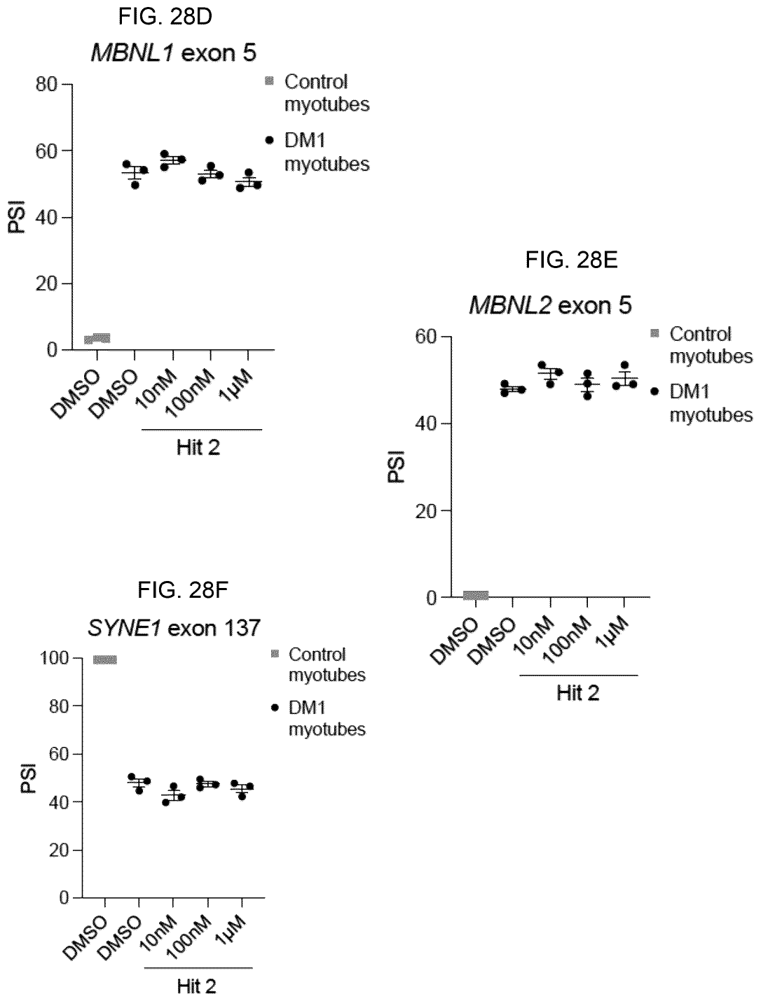

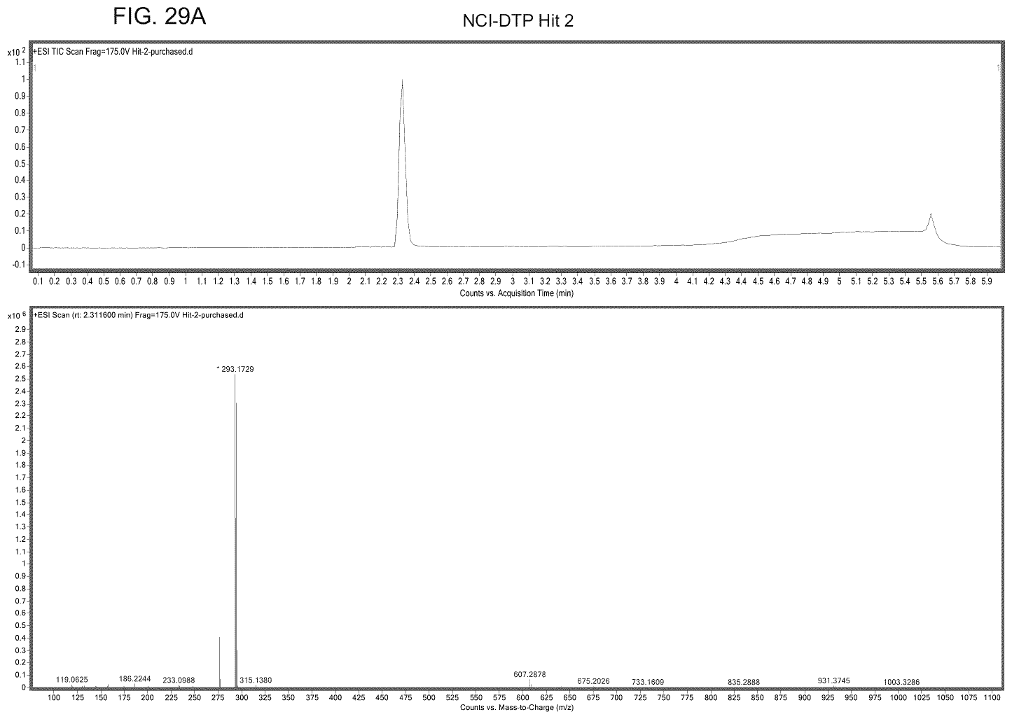

We next investigated the effects of Hit 2 on expression of ATXN genes in control fibroblasts at the doses where we saw maximal reduction in expression of ATXN genes in patient derived fibroblast lines. In two of the three control fibroblast lines, no effect on ATXN1 expression with Hit 2 was observed. For the third cell line (Control 3) a 12.8% reduction of ATXN1 expression was observed at 100 nM (P=0.0424; FIG. 5I). No measurable reduction of ATXN3 expression was observed at 10 nM or 100 nM concentration in the three control cell lines (FIG. 5J). Interestingly, there was not a consistent effect of Hit 2 on ATXN7 expression across multiple doses in three control fibroblast lines (FIG. 28A) or in SCA7 patient derived fibroblast lines (FIG. 28B).

Together, these data indicate that Hit 2 regulates CAG transcript levels in the context of multiple SCA disease genes and does so in a repeat expansion selective manner. As Hit 2 is predicted to bind CUG repeat RNA, although with lower affinity than CAG repeat RNA, we wanted to understand whether Hit 2 could reduce expression of CUG expansion transcripts in cell culture. To do this, we differentiated DM1 patient derived myoblasts into myotubes and performed a three-day treatment of Hit 2.

Increasing doses of Hit 2 showed a trend to increase expression of DMPK, the CUG repeat expansion containing gene causative for DM11, with 1 μM Hit 2 increasing DMPK expression by 51.6% (P=0.0266; FIG. 28C). To understand if Hit 2 is capable of rescuing CUG induced mis-splicing which is a transcriptomic hallmark of DM11, we performed RT-PCR analysis and found that Hit 2 did not affect the splicing of three alternatively spliced events in DM1 myotubes (FIGS. 28D-F). This therefore demonstrates that the mechanism of action of Hit 2 is specific to CAG repeats, consistent with in silico docking predictions (FIGS. 41-4L); possibly indicating that Hit 2 does not bind CUG repeats strong enough to reduce expression of CUG repeat containing transcripts or to displace the MBNL RNA binding proteins responsible for CUG induced mis-splicing.

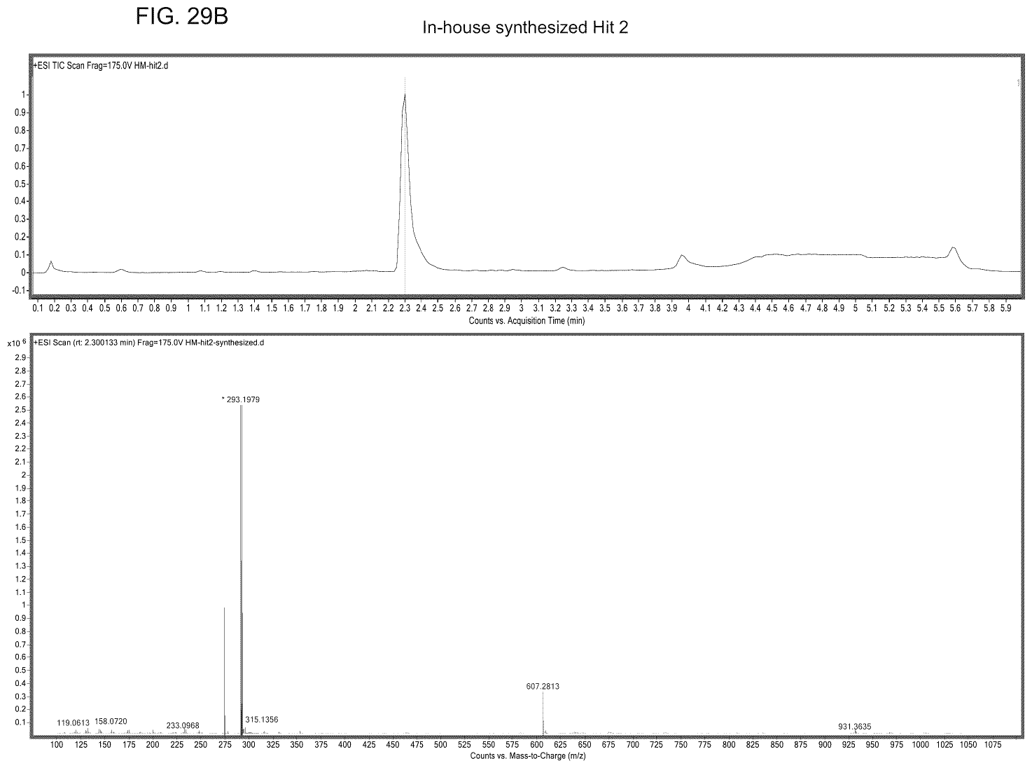

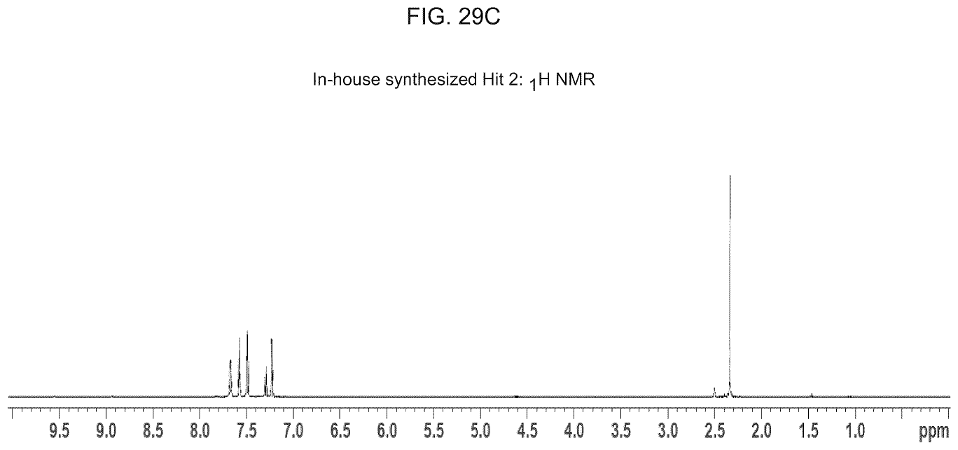

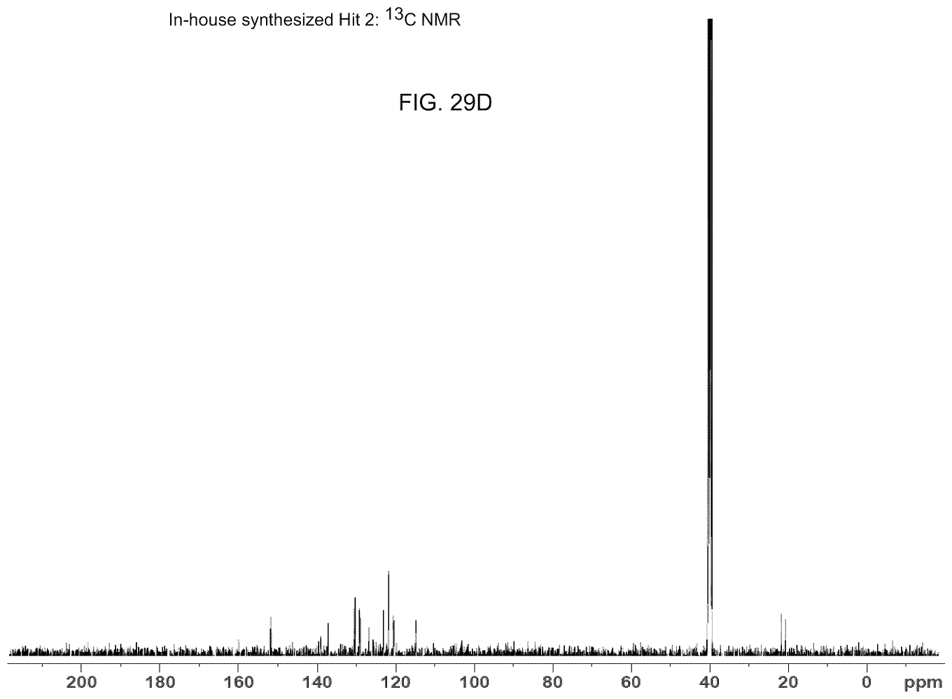

Lead candidate compound Hit 2 is well tolerated in vivo and reduces expression of CAG expansion transcripts in the Atxn1154Q/2Q SCA1 mouse model. Given that Hit 2 was well tolerated in patient derived fibroblast cell lines and selectively reduced ATXN1 and ATXN3 expression levels in a repeat expansion selective manner, we investigated if Hit 2 was capable of reducing expression of CAG SCA disease associated genes in vivo. Because of the large effect size seen in SCA1 fibroblasts, we decided to perform the in vivo treatment of Hit 2 in an SCA1 mouse model and selected the Atxn1154Q/20 knock-in model to ensure we were investigating Atxn1 expression under endogenous control. To generate sufficient quantity of Hit 2 for an in vivo study, we synthesized Hit 2 in house as previously described (FIG. 29). Animal model studies using Hit 2 have not previously been reported and so with this study, we set out to perform a short two-week treatment regimen at 25 mg/kg (<4m/kg DMSO) and to closely monitor the mice for signs of toxicity during this time frame.

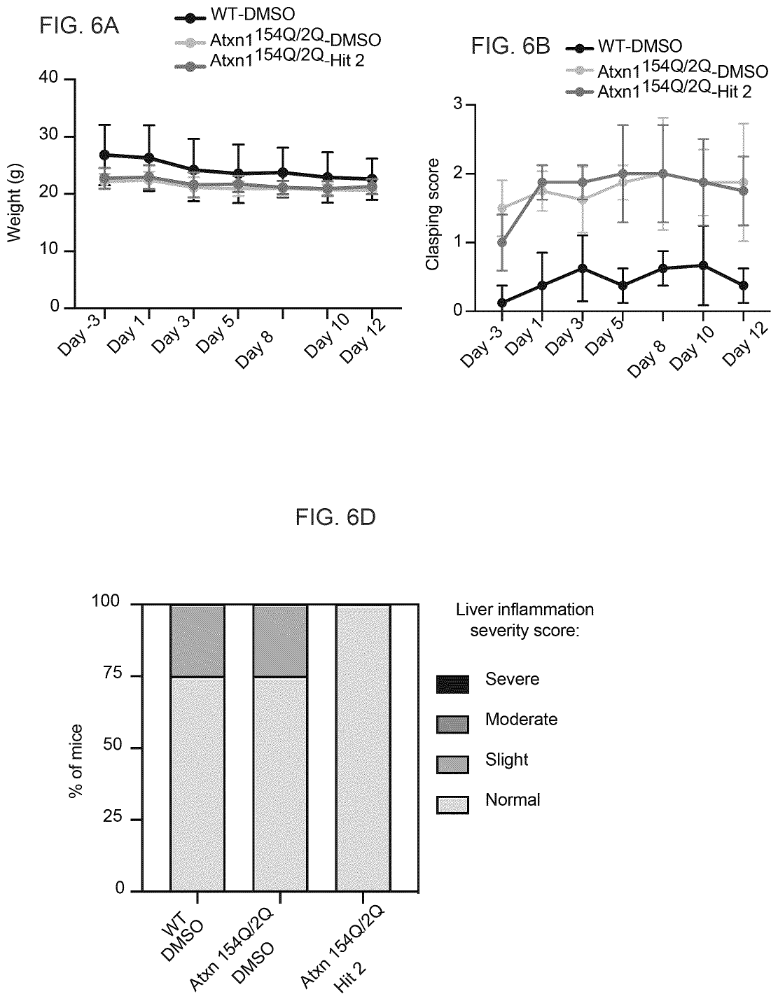

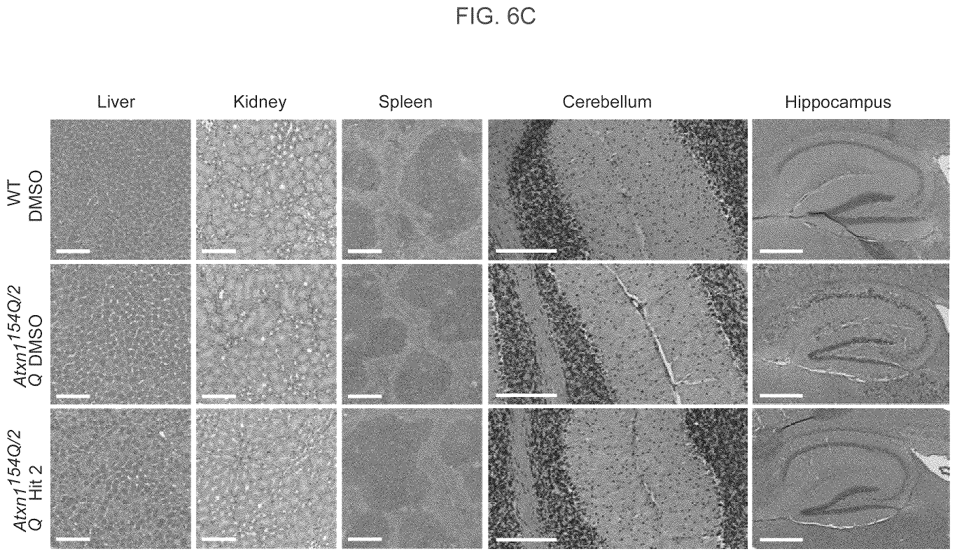

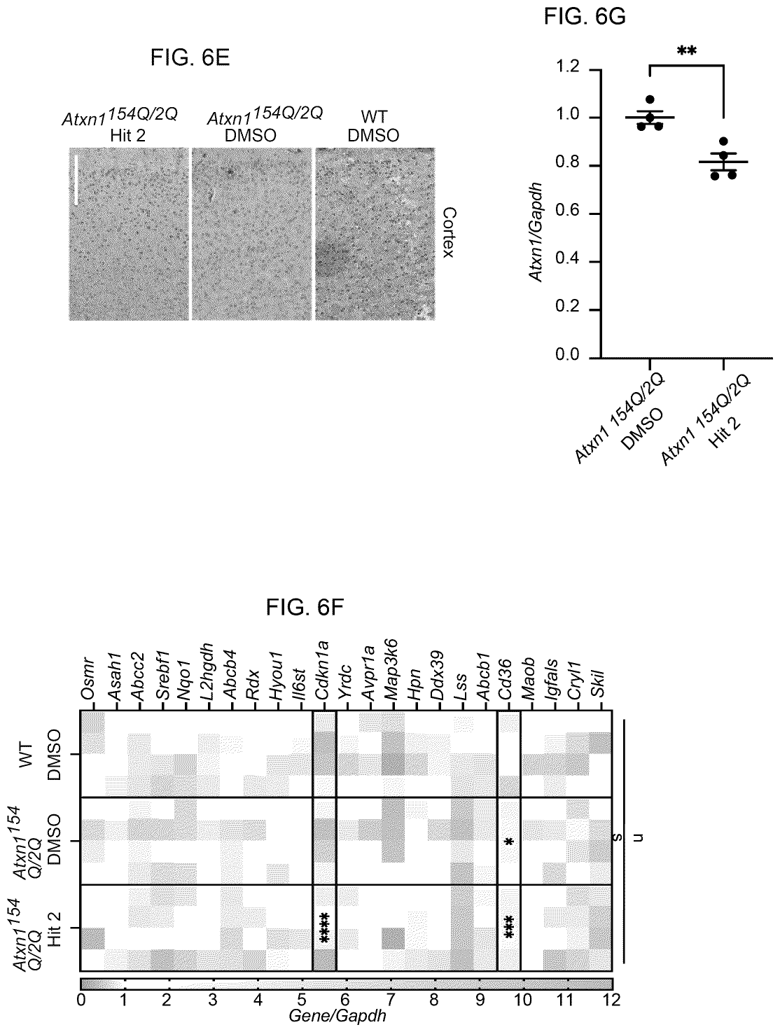

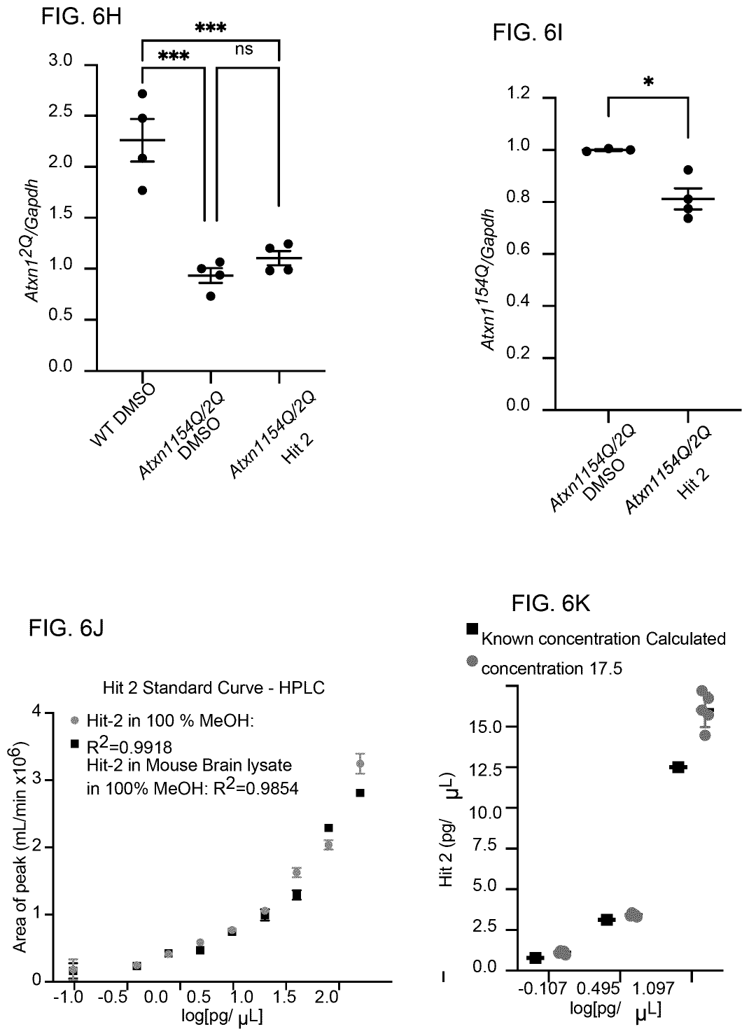

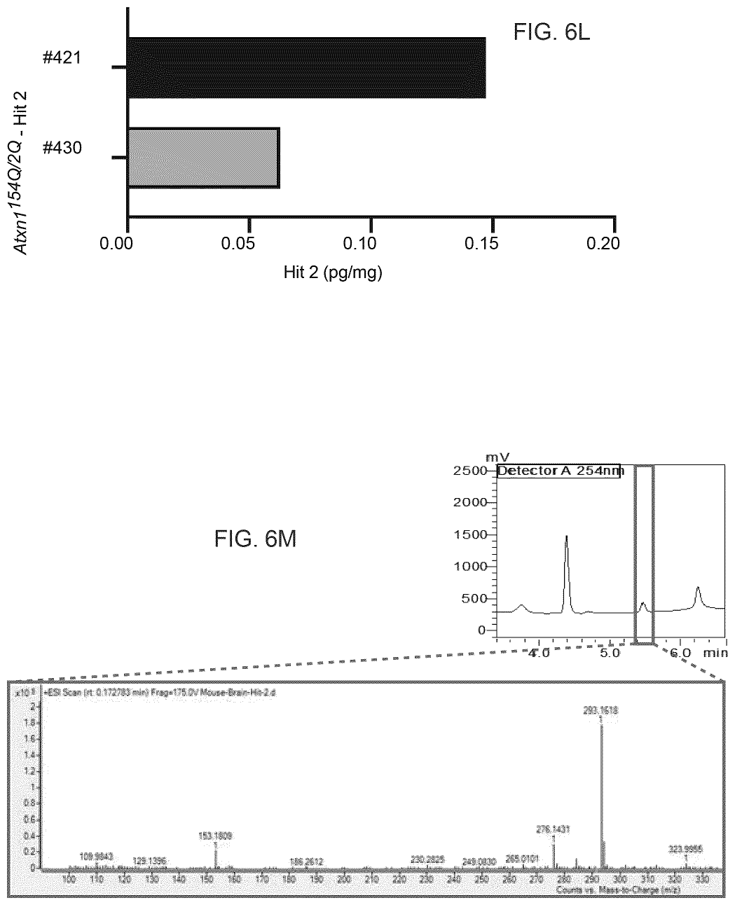

Starting at 18 weeks of age, Hit 2 or DMSO was injected via IP for 5 days, followed by two rest days and then an additional 5 days of IP injection before sacrificing the mice. Across the treatment timeline, Hit 2 did not negatively impact mouse weight beyond the effects seen for DMSO alone (FIG. 6A) and there was no negative impact of Hit 2 on the clasping phenotype of Atxn1154Q/2Q mice compared to DMSO injected mice (FIG. 68).

Together these data provide the first indication that Hit 2 is well tolerated in vivo over a short study time frame at 25 mg/kg. Following completion of the two-week injection regimen, to further investigate the in vivo toxicity and safety profile of Hit 2, we performed hematoxylin and eosin staining of liver, kidney, spleen and sagittal brain sections (FIG. 6C). No gross morphological changes were seen across the tissues investigated (FIG. 6C) and analysis of morphological hepatic features showed that macrovesicular and microvesicular steatosis, fibrosis, single cell apoptosis, necrosis, karyomegaly, pigment and glycogen accumulation were absent from all mice in the study. One wild-type mouse and one DMSO treated SCA1 mice showed signs of slight inflammation in the liver demonstrating that there are no significant hepatic morphological changes due to administration of Hit 2 (FIG. 6D); consistent with this, immunostaining for GFAP, a marker of reactive astrogliosis, demonstrated that Hit 2 treatment does not induce neuroinflammation (FIG. 6E).

Finally, we performed qPCR for 24 markers of hepatotoxicity and saw that there was no treatment group dependent changes in gene expression (P=0.2360), with treatment group explaining only 0.92% of variation in the data and the expression profiles of different genes explaining 15.28% of variation in the data (FIG. 6F). One Hit 2 treated mouse showed an 11.79-fold pregulation of Cdkn1a which did not correlate with pathological abnormalities in the liver but resulted in Cdkn1a showing a significant increase in expression compared to wild-type and DMSO injected SCA1 mice (P<0.0001). Interestingly. Cd36 showed a 2.5-fold upregulation in DMSO injected Atxn1154Q/2Q mice (P=0.016) and a 3-fold upregulation in Hit 2 injected Atxn1154Q/20 mice (P=0.0004) compared to wild-type mice (FIG. 6F). Together, these data demonstrate that Hit 2 is well tolerated in vivo and does not induce inflammation or toxicity beyond the effects of DMSO alone.

To understand if Hit 2 was capable of reducing Atxn1 transcript levels in vivo, we performed RNA extraction on cerebellum from Hit 2 and DMSO treated SCA1 and WT mice. Compared to DMSO injected Atxn1154Q/2Q mice, Hit 2 reduced expression of Atxn1 in the cerebellum by 18.5% (P=0.0056; FIG. 6G). To understand if this reduction was specific for the expansion allele, we utilized allele specific qPCR primers to differentiate between the 2Q and 154Q alleles. As expected, DMSO injected WT mice showed approximately twice the expression of Atxn12Q compared to SCA1 mice: 132.6% increase compared to Atxn1154Q/2Q-DMSO (P=0.0002) and 115.7% increase in Atxn12Q compared to Hit 2 treated SCA1 mice (P=0.0005), consistent with previous studies.

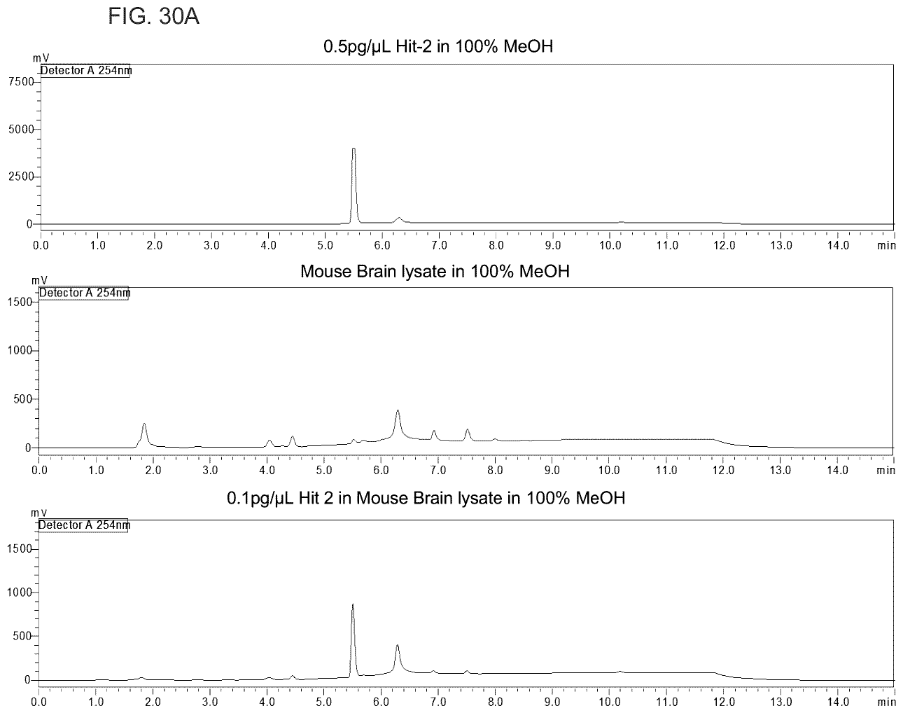

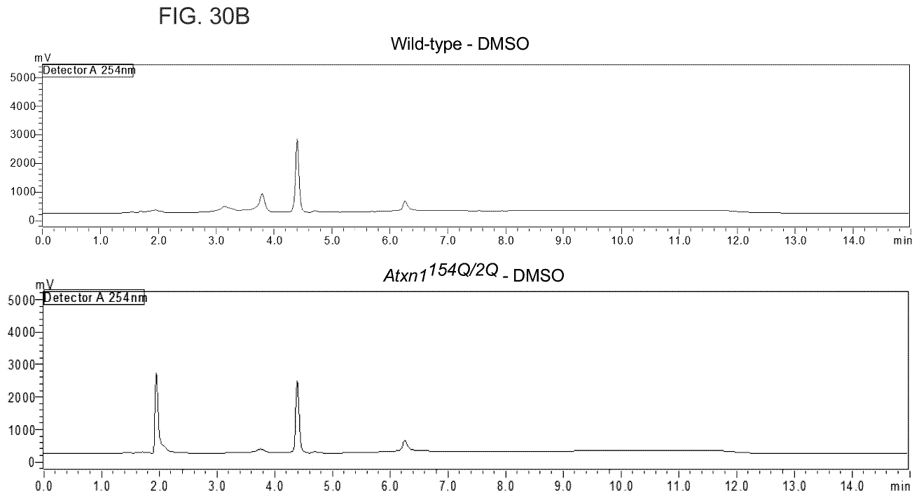

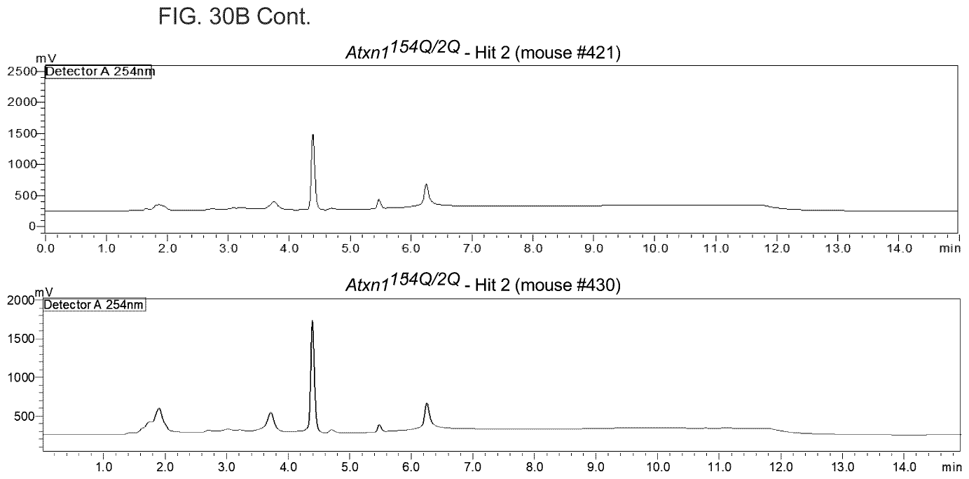

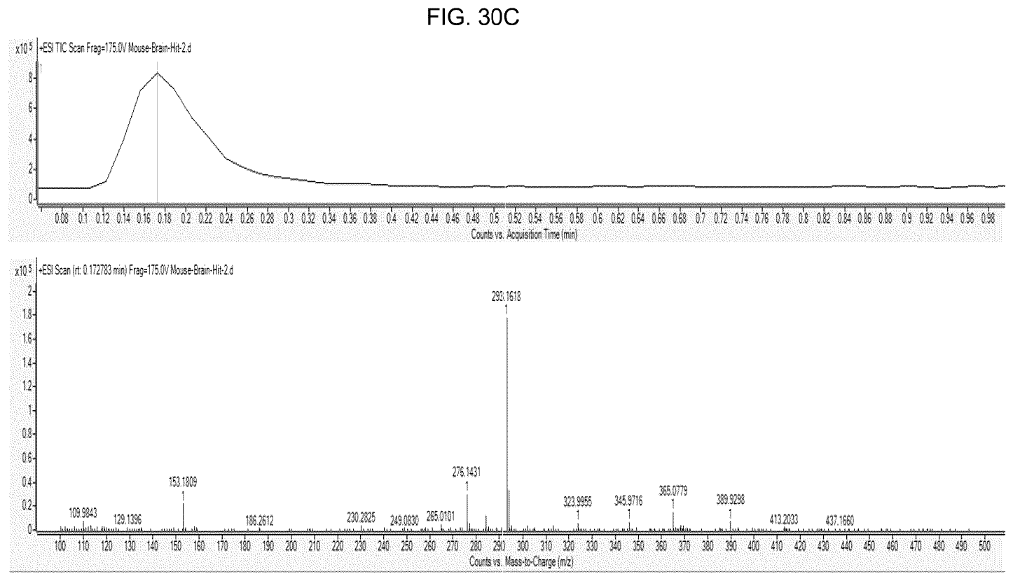

There was however no difference in Atxn12Q expression between DMSO and Hit 2 injected SCA1 mice (FIG. 6H). This is in contrast to the Atxn1154Q allele selective qPCR which showed a 18.8% reduction in expression of Atxn1154Q in Atxn1154Q/2Q-Hit 2 mice compared to DMSO injected (P=0.0109; FIG. 6I); thereby demonstrating that over a two-week period, Hit 2 can selectively reduce expression of expansion Atxn1 alleles in vivo. Having observed an effect of Hit 2 on expansion transcript levels in the brain of SCA1 mice, we next wanted to confirm that Hit 2 could be detected in Atxn1154Q/20-Hit 2 mouse brain but not in DMSO injected mice. To do this, we developed a novel HPLC method to detect and quantify Hit 2 in vivo. By utilising Hit 2 at known concentrations in 100% methanol we were able to generate a standard curve with an R2 of 0.9918 (FIG. 6J). We next lysed wild-type mouse cortex and midbrain in 100% methanol and spiked in Hit 2 at the same concentrations and were able to generate a standard curve with an R2 of 0.9854 and a very similar trajectory as Hit 2 in 100% methanol (FIG. 6J, and FIG. 30A).

Across all concentrations a peak for Hit 2 was observed at a retention time of 5.4-5.6 mins, no peak was observed for mouse brain lysed in 100% methanol (FIG. 30A). To validate the accuracy of calculating concentrations based on the exponential equation of the standard curve generated from Hit 2 in lysed mouse brain, we ran three samples of known concentration an additional five times and found that at lower concentrations (0.78 and 3,125 pg/μL) the calculated concentration showed high accuracy (1.112 pg/μL*0.098 and 3.415 pg/μL*0.091 respectively) but by 12.5 pg/l there was overestimation of the concentration (16.03 pg/μL±1.05: FIG. 6K). Utilising this method, HPLC traces for midbrain and cortex from DMSO injected wild-type and Atxn1154Q/2Q mice did not show a peak at 5.4-5.6 mins but two Hit 2 injected Atxn1154Q/2Q mice showed a clear peak (FIG. 308) corresponding to concentrations of 0.0624 and 0.1469 pg Hit 2 per mg of brain tissue lysed (FIG. 6L). Finally, to confirm that the peak at 5.4-5.6 min corresponded to Hit 2 in Hit 2 injected mice, we collected this fraction for mouse 421 and subjected it to LC/MS where we observed a peak at m/z=293.1618 corresponding to the molecular weight of Hit 2[292.346+H+j (FIG. 6M, and FIG. 5C). Together these data demonstrate that Hit 2 is well tolerated in vivo over a short-term study and can be detected in the brain of treated mice where it selectively reduces expression of expanded CAG transcripts.

Hit 2 rescues dysregulation of alternative splicing in the Atxn1154Q2Q SCA1 mouse model: To understand more about the effects of Hit 2 in vivo we performed RNA-Seq on cerebellum from WT-DMSO, Atxn1154Q/2Q-DMSO and Atxn11540/20-Hit 2 treated mice (Table S1). Recently, we and others reported that dysregulation of alternative splicing is a widespread transcriptomic hallmark of CAG expansion SCAs including in the Atxn1154Q/2Q mouse model. Furthermore, we demonstrated that alternative splicing could be used as a target engagement biomarker for CAG SCA. We therefore set out to assess the effect of Hit 2 on both gene expression and alternative splicing. Because previous alternative splicing analyses in CAG SCAs were performed on datasets generated using polyA selection and our standard pipeline for alternative splicing analysis is to use rRNA depletion, we first assessed whether our in house dataset recapitulated the published alternative splicing studies. Of the significant mis-spliced events between WT-DMSO and Atxn1154Q/2Q-DMSO mice with a change in percent spliced in (PSI)>10% and FDR<0.1, skipped exon (SE) events were the most frequently dysregulated, accounting for 50.1% of all events (FIG. 31A).

Dysregulation of both inclusion and exclusion events were detected with a mean PSI for inclusion events of 23.0% and a maximum PSI of 92.0%; for exclusion events these values were 19.4% and 58.4% respectively (FIG. 31B). Gene ontology enrichment analysis revealed that skipped exon events occurred in genes implicated in nuclear processes (eg GO:0051647), synapse organization (eg GO:0050807), calcium ion regulation and membrane potential (eg GO:0048791and R-MMU-112308; FIG. 31C). While all these findings are consistent with the extent of splicing dysregulation and the pathways affected previously reported 34,36, we also confirmed dysregulation of Trpc3 exon 9 (FIG. 31D) and Anks1b exon 5 (FIG. 31E) SE events that were previously reported.

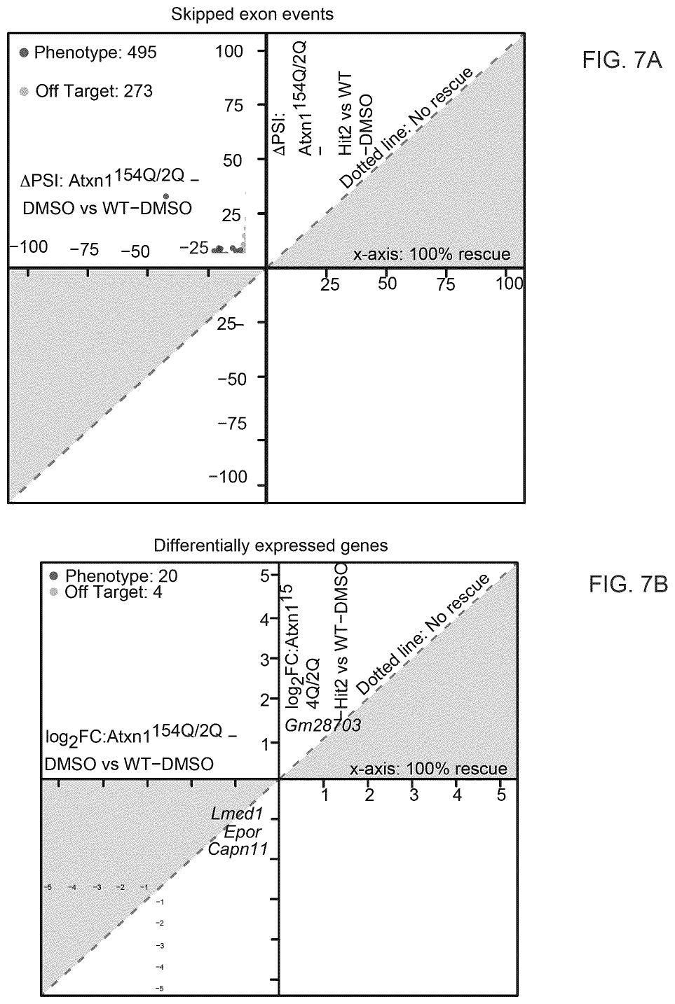

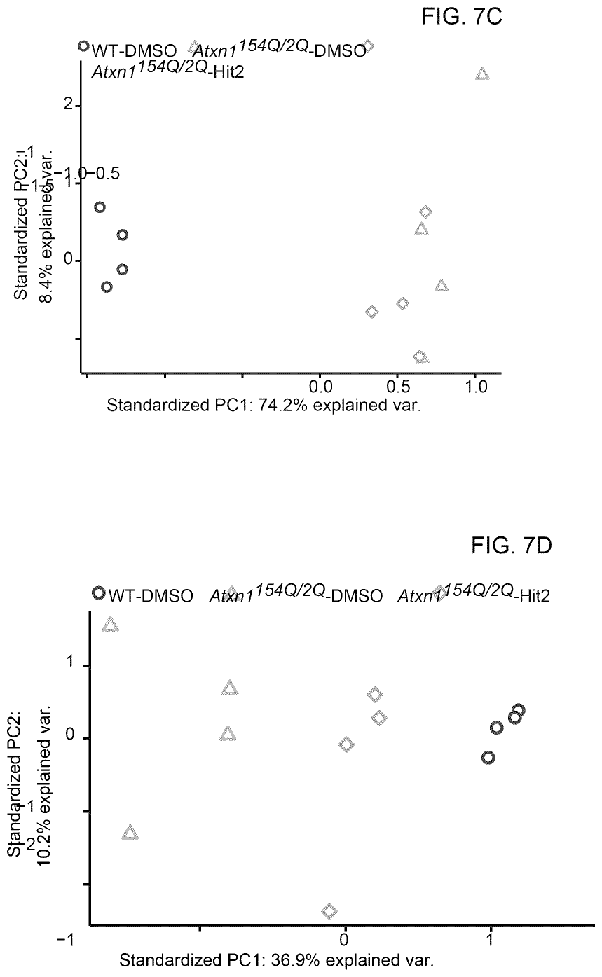

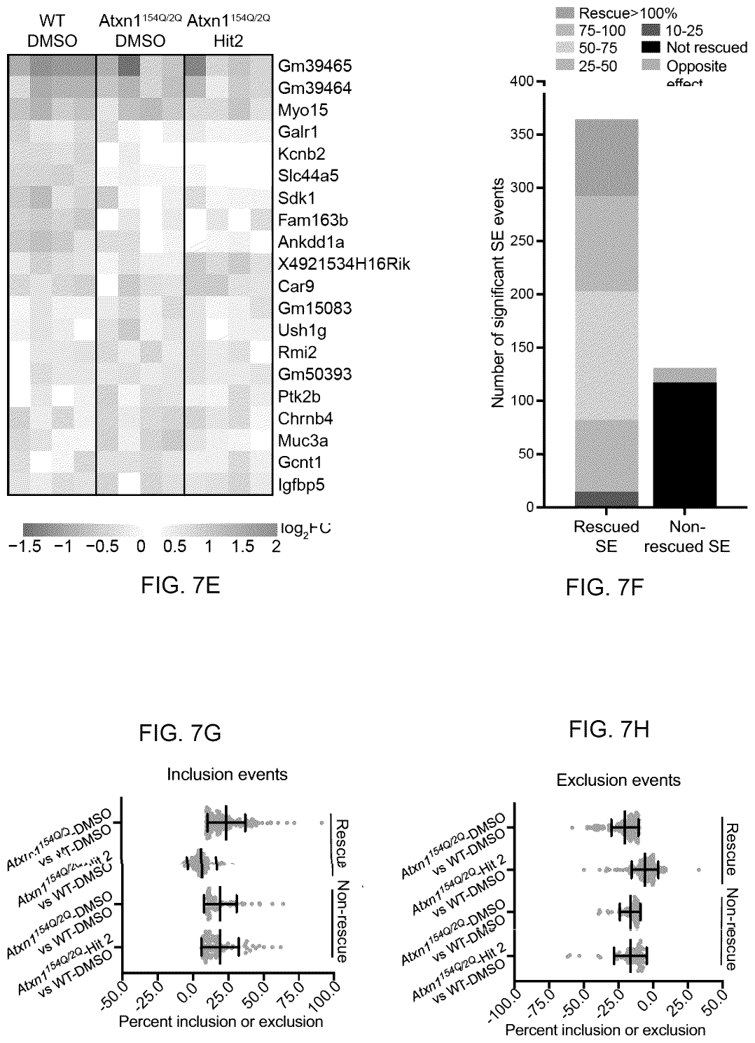

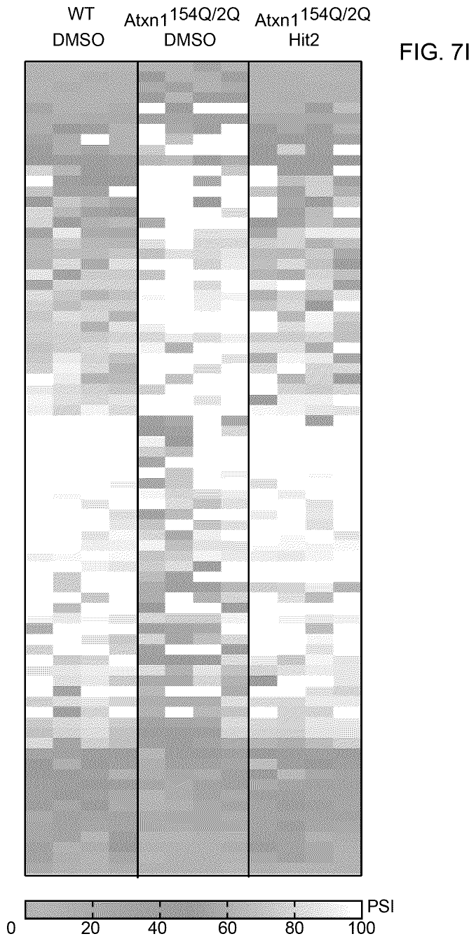

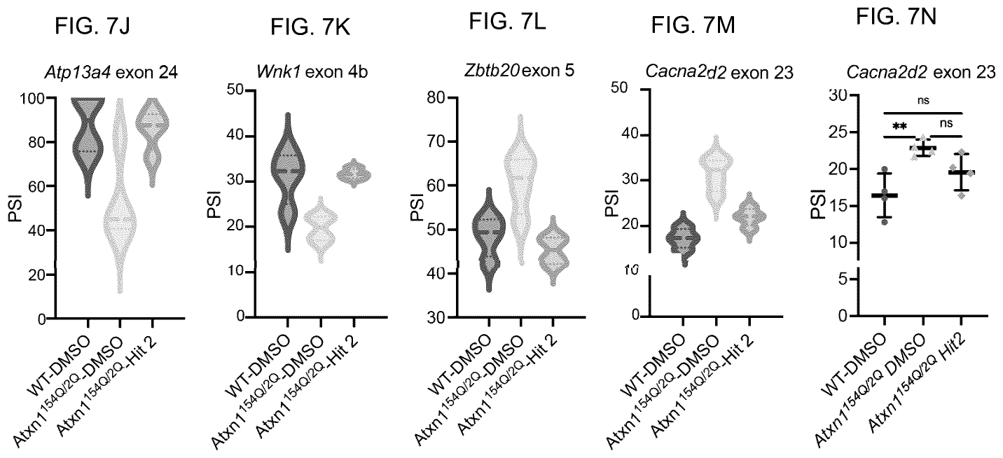

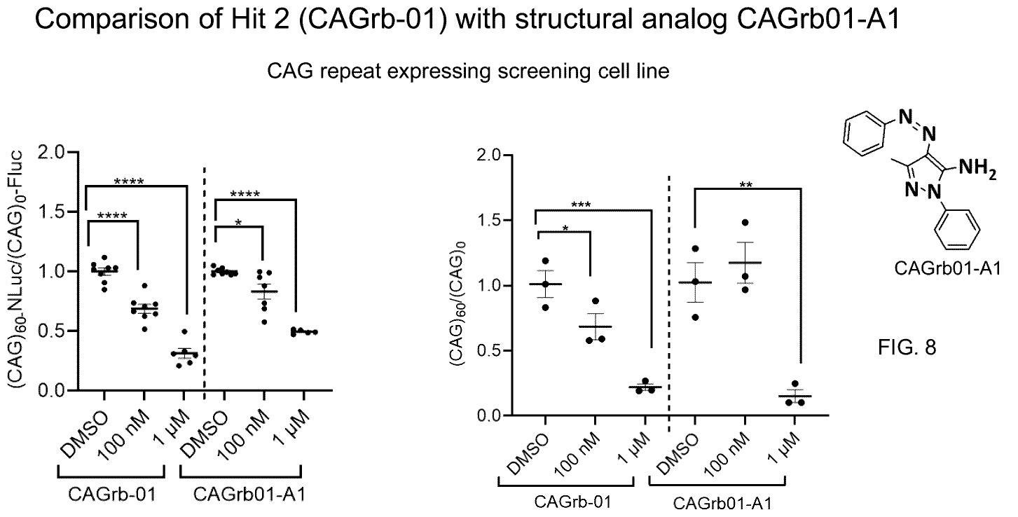

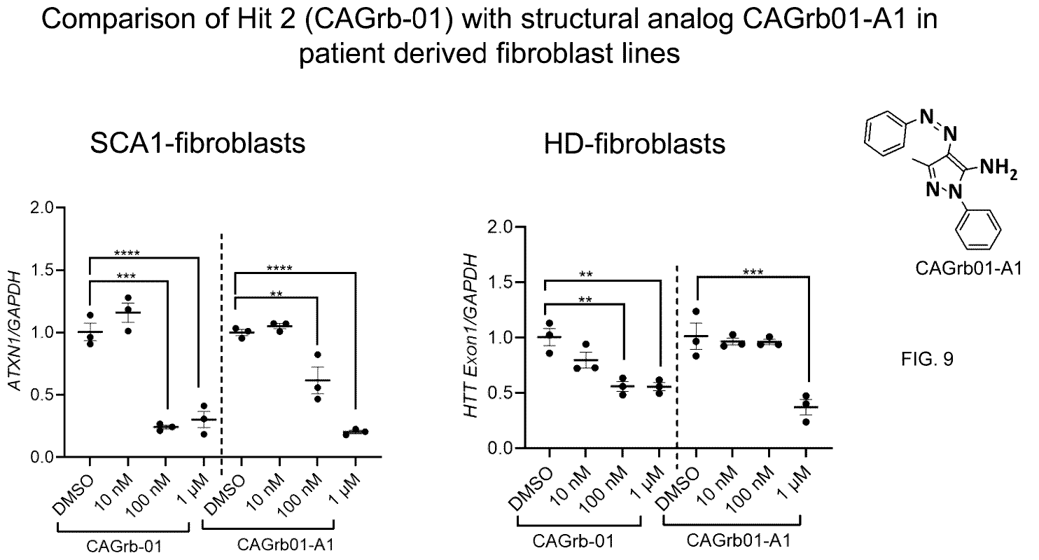

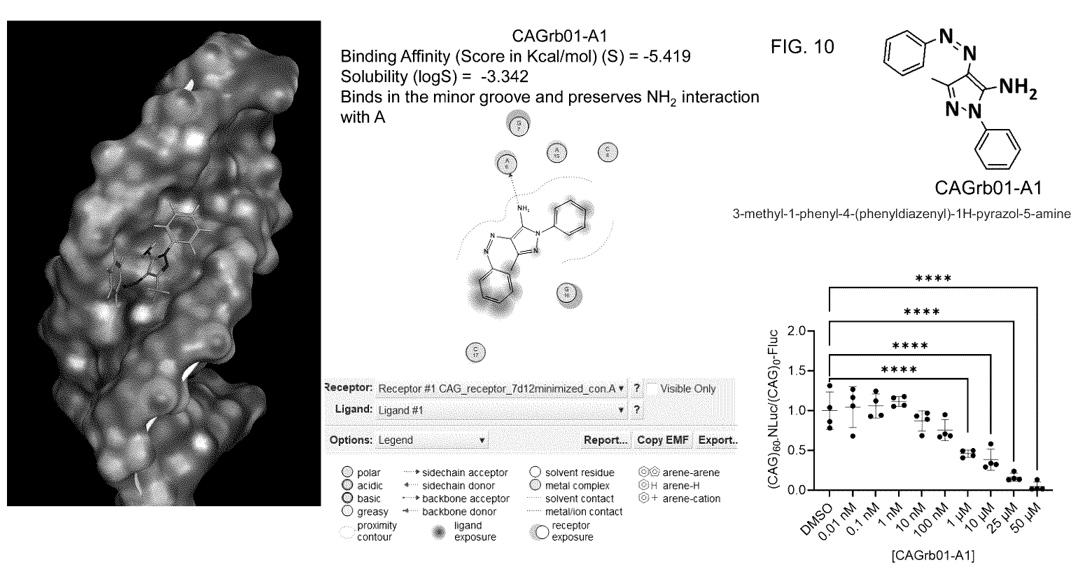

Together, these data independently validate that dysregulation of alternative splicing is a prominent transcriptomic feature affecting disease relevant pathways in CAG expansion SCAs. We next sought to understand whether Hit 2 impacted alternative splicing and differential gene expression in the Atxn1154Q/2Q mouse model. Because SE events accounted for 50.1% of all miss-spliced events, we focused our alternative splicing analysis on SE events. Of the 495 SE events dysregulated between WT-DMSO and Atxn1154Q/2Q-DMSO mice, the majority showed smaller differences between WT-DMSO and Atxn1154O120-Hit 2 with a trend towards overall rescue of events. We also detected 273 SE events dysregulated between WT-DMSO and Atxn1154Q12Q-Hit 2, but not between WT-DMSO and Atxn1154Q/2Q-DMSO; demonstrating an off-target effect of Hit 2 accounting for 0.59% of all SE events detected (FIG. 7A).

Interestingly, far fewer differentially expressed genes were detected (20 genes; log 2FC>1, padj<0.05) with most events clustering around the dotted line indicating no rescue. Four events were differentially expressed between WT-DMSO and Atxn11540/2Q-Hit 2 but not WT-DMSO and Atxn1154Q/2Q-DMSO indicating minimal off target effects of Hit 2 on differential gene expression (FIG. 7B); this is consistent with the lack of changes in expression seen for hepatotoxicity markers in the liver (FIG. 6F).

The limited rescue of differential gene expression compared to strong rescue of alternative splicing by Hit 2 is exemplified by PCA plots based on significant events between WT-DMSO and Atxn1154Q/20-DMSO (FIGS. 7C, and 7D). For differential gene expression, there is clear separation between WT and SCA1 mice but overlap between the Hit 2 and DMSO injected SCA1 mice (FIG. 7C) indicating a lack of rescue of differentially expressed genes which is also clearly seen at the level of individual events (FIG. 7E). In contrast, the PCA plot for skipped exons shows clear separation between all three treatment groups along the x-axis accounting for 36.9% of variance, with Atxn1154Q/20-Hit 2 mice approximately half-way between WT-DMSO and Atxn11540/20-DMSO mice (FIG. 7D).

These data therefore show that Hit 2 rescues dysregulation of alternative splicing in Atxn1154Q/2Q mice with limited off target effects and without impacting differential gene expression. To further understand the extent of alternative splicing rescue, we categorized the 495 SE events (FIG. 7A, and FIG. 31A) based on the size of change (percent rescue) between Atxn1154Q/2Q-DMSO and -Hit 2. Events were classified as rescued if the PSI for Atxn1154Q/2Q-Hit 2 changed>10% in the direction of the control mice and had a □PSI>5%, compared to Atxn1154Q/2Q-DMSO. Events were classified as changed in the opposite direction if DPSI>5% for Atxn1154Q/2Q-Hit 2 versus-DMSO and there was >10% shift in the opposite direction to the control mice; the remainder of events were classified as not rescued. Of the 495 SE events 364 (73.5%) were rescued with 282 rescued by >50%; only 14 events (2.83%) showed an opposite effect while 117 events (23.6%) remained unchanged (FIG. 7F). For rescued inclusion events, the mean DPSI compared to WT-DMSO reduced from 24.4% for Atxn1154Q/2Q-DMSO to 6.73% for Atxn1154CY2Q-Hit 2 with a corresponding-30% change in PSI for the minimum and maximum PSI values (FIG. 7G). Likewise, for rescued exclusion SE events, the mean DPSI shifted from −20.4% for WT-DMSO vs Atxn1154Q/2Q-DMSO to −5.83% for WT-DMSO vs Atxn1154Q/2Q-Hit 2 with an 8.55% shift in the minimum DPSI and a 43% shift in the maximum DPSI (FIG. 7H).

Next, we further filtered these SE events to identify events significantly alternatively spliced between Atxn1154012Q-DMSO and -Hit 2 mice (FIG. 7I). Many of these events affect genes involved in disease relevant pathways, for example cellular calcium ion homeostasis (Atp13a4, FIG. 7J), a serine/threonine protein kinase with links to hereditary neuropathy (Wnk1, FIG. 7K), transcription factors (Zbtb20, FIG. 7L), and a voltage-dependent calcium channel linked to cerebellar atrophy (Cacna2d2, FIG. 7M). Using RT-PCR, we confirmed that Cacna2d2 exon 23 shows significantly increased inclusion at 12 (5.96%. P=0.0005) and 23 (8.77%, P=0.0004) weeks of age in Atxn1154Q/2Q mice compared to WT mice (FIG. 31F) and that this event is rescued following Hit 2 treatment (FIG. 7N). In summary, Hit 2 reduces the severity of the transcriptomic phenotype in Atxn1154Q/2Q mice with minimal off target effects.

There is currently a large unmet therapeutic need for spinocerebellar ataxias caused by CAG repeat expansions. Despite recent progress in preclinical therapy development, and ongoing clinical trials using ASOs for these devastating diseases, there remains a gap in the development of small molecule therapeutics that can target the shared pathogenic source, the CAG repeat expansion, across this class of SCAs.

This is reflected in the lack of small molecules published to reduce expression of CAG expansion transcripts in SCAs. To address this gap, we developed a disease-gene independent, mechanism-agnostic CAG repeat selective small molecule screening cell system that recapitulated phenotypic and transcriptomic hallmarks of mouse models of CAG expansion SCAs.

Utilizing this cell line, we Identified that the FDA approved microtubule inhibitor, colchicine was capable of selectively reducing expression of CAG expansion transcripts relative to a zero-repeat control, In SCA1, SCA3 and SCA7 patient derived fibroblasts, colchicine was able to reduce expression of the disease-causing transcripts in a repeat dependent manner. By screening 1584 structurally diverse small molecules, we identified a pyrazole based compound, Hit 2 (IUPAC name: 4-[(4-methylphenyl)diazenyl]-1-phenylpyrazole-3,5-diamine; CAS number: 5456-92-8), that selectively reduced expression of CAG expansion transcripts and polyglutamine expansion proteins relative to zero repeat controls. Hit 2 reduced levels of ATXN1 and ATXN3 transcripts in SCA1 and SCA3 patient derived fibroblast lines but not in control fibroblast lines, and Hit 2 reduced ATXN1 and ATXN3 protein levels in SCA1 and SCA3 patient cell lines.

Hit 2 was also shown to selectively reduce expression of expansion alleles and rescue disease-associated alternative splicing dysregulation in a knock-in mouse model of SCA1 with minimal off target effects on the transcriptome. Furthermore, we have recently demonstrated that Hit 2 rescues missplicing in SCA1 and SCA3 patient derived fibroblast lines. This first in mouse study of Hit 2 was therefore able to not only show the safety of Hit 2 in vivo but also independently validated our recent data demonstrating the feasibility and validity of assessing alternative splicing dysregulation as a target engagement biomarker in CAG expansion SCA. Interestingly, colchicine and Hit 2 were identified to regulate CAG expansion transcripts through distinct mechanisms with Hit 2 able to interact with CAG repeat RNA. This therefore demonstrates that our novel small molecule screening system is capable of identify compounds with different mechanisms of action for regulating expression of CAG repeat expansions.

Previous mechanism-agnostic small molecule screening approaches have also identified compounds with therapeutic promise in SCAs, primarily focusing on SCA337-40. Multiple cell-based screening systems have been developed to identify compounds capable of regulating expression of ATXN3 protein based on a luciferase readout. One of these screening systems identified three compounds, including the FDA approved atypical antipsychotic agent aripiprazole, capable of reducing expression of human ATXN3-84Q and mouse Atxn3 protein in organotypic brain slice cultures from 840 SCA3 transgenic mice 37. Interestingly however, another cell based screening system did not identify compounds capable of reducing ATXN3 expression but found that statins upregulate expression of ATXN3 mRNA and protein levels; providing valuable insight into management of SCA339. Other screens in Caenorhabditis elegans models of SCA3 identified compounds capable of improving behaviour phenotypes and found that the FDA approved selective serotonin reuptake inhibitor, citalopram was capable of reducing mutant ATXN3 aggregation in SCA3 mouse models.

While these screening systems highlight the ability of mechanism agnostic screening approaches to identify compounds that can regulate polyglutamine containing protein behaviour, many of these compounds act on both normal and expanded ATXN3 and do not reduce expression of ATXN3 mRNA.

In contrast to these studies, our screening approach set out with a broader aim to identify compounds with therapeutic potential across CAG SCAs. Other groups have sought to identify candidate therapeutics that could be used across multiple CAG SCAs through their ability to rescue cellular processes commonly disrupted in SCAs. For example, as Purkinje cell function is impacted in many SCAs, therapies that target this, such as 4-aminopyridine and chlorzoxazone, may provide therapeutic benefit across a range of SCAs. Similarly, Trehalose, a compound that has been shown to induce autophagy, has shown promise in models of SCA3 and SCA17, and is currently in clinical trials for SCA. Additionally, more general neuroprotective compounds such as riluzole and its prodrugs are in clinical trials for multiple CAG SCAs, but so far have shown mixed results.

While these approaches may result in therapeutic benefit across multiple SCAs, the window of benefit is likely restricted due to the ongoing neurodegenerative effects caused by the disease-causing genes which cannot be targeted using these approaches. We have been able to identify compounds that target the pathogenic source of CAG expansion diseases and may yield therapeutic benefit across multiple CAG SCAs. By integrating a repeat-selective aspect into a gene-context independent screening system we selected for compounds that retained repeat selectivity in more complex and disease relevant model systems of multiple CAG SCAs. Although the compounds retained allele selectivity at the RNA level, effects were seen for both expanded and control alleles for ATXN3 protein reduction for Hit 2 suggesting that further investigation is needed to understand the repeat length thresholds for effects at the RNA versus protein levels for Hit 2.

Despite the strengths of our novel screening system, it has limitations: primarily a lack of ability to control expression from the integrated constructs. In combination with the high level of cell toxicity associated with expressing CAG repeats in this system, the lack of ability to control expression results in selective pressure against both the length and the expression level of the pure CAG repeat. Reengineering this system into an inducible system would reduce the selective pressure against expression of the integrands and increase the longevity of an individual clonal cell line as well as providing a more tractable model system for investigating the effects of expression of CAG repeat expansions.

Multiple previous screening systems in CAG SCAs and indeed our own study, identified FDA approved compounds with therapeutic potential in CAG SCAs. Repurposing of FDA approved compounds is designed to accelerate the pace of therapeutic development by advancing candidates ready for human trials that do not require extensive pharmacodynamic and/or toxicity studies. Here we identified the FDA approved microtubule regulating compound colchicine as a CAG repeat containing transcript regulating compound. One of the draw backs of colchicine in this study is that it was unable to reduce expression of the polyQ proteins in our screening cell line. This raises the idea of combinatorial therapies based on targeting different levels of the pathogenic cascade or combining therapies with different mechanisms of action to reduce the dose of each therapy and thereby reduce unwanted toxicity.

In the context of combining small molecule approaches with ASOs this could potentially help overcome issues of toxicity whilst retaining effect size of reducing CAG expansion containing transcripts. If paired with non-allele selective ASO approaches, repeat selective small molecules could also aid in preventing excessive reduction of expression of the non-expanded protein levels. Similarly, given that both the FDA approved compounds citalopram and aripirazole have been shown to reduce levels of ATXN3 protein but not mRNA, it would be interesting to understand if, at least in the context of SCA3, there was an additive effect of either compound in combination with colchicine, or Hit 2, which regulate CAG containing mRNA. Interestingly, another microtubule regulating compound, nocodazole, was identified through a screen in SCA6 for its ability to prevent internal ribosome entry site (IRES) mediated translation of the a1ACT protein product which is toxic in the context of SCA6. It remains to be seen whether nocodazole also regulates CAG expansion expression in a similar manner to colchicine. However, the rationale behind using microtubule regulating compounds in neurodegenerative diseases in which the cytoskeleton and microtubule associated processes are disrupted is questionable and may accelerate degeneration through distinct mechanisms rather than slow the disease process.

Furthermore, although other microtubule regulating compounds may be tolerated better, colchicine is typically prescribed on a short-term basis due to the effects of colchicine poisoning. This is a toxicity syndrome that can lead to death and in some cases causes neuropathy, myopathy, distal sensory abnormalities, and nerve conduction impairment and so colchicine does not represent a good therapeutic for neurodegenerative conditions.

Hit 2 represents a promising starting point for developing a novel therapeutic compound for CAG repeat expansion diseases because it is capable of reducing both CAG expansion transcript and polyQ protein expression. Despite the current problems of insolubility of Hit 2 and the requirement of DMSO or methanol as a solvent, understanding the binding mode and mechanism of action of Hit 2 will enable us to perform structure activity relationship studies to identify structural analogs of Hit 2 with improved solubility and more druglike properties.

Although our first in mouse experiment of Hit 2 demonstrated that it was well tolerated over a two-week period and pathological and transcriptional readouts demonstrated no overall toxicity or inflammation, the mice in the study did not gain weight due to the daily DMSO administration, and so a compound with improved solubility will be required before a long-term behaviour, electrophysiology and survival study can be performed. Despite this, our short-term study demonstrated promising effects of reducing Atxn1 expression levels in a repeat selective manner and rescuing transcriptomic hallmarks of disease.

Analogs of Hit 2 will also enable a better understanding of the relative effects of CAG RNA binding compounds on differential gene expression and alternative splicing transcriptomic hallmarks as well as on ATXN protein expression in CAG SCA mice. During development of Hit 2, it will be important to consider the effects on CUG expansion transcripts due to the known role of antisense transcripts in repeat expansion diseases. Here we found that Hit 2 increased expression of DMPK in DM1 myotubes. If this increase in expression also occurs for antisense CUG expansion transcripts in CAG SCA disease causing genes, this could lead to disease perpetuating off-target effects.

With respect to identifying structural analogs of Hit 2 having improved properties, FIG. 4I indicates that Hit 2 is predicted to interact with the minor groove of the CAG repeat RNA. Hydrogen bonds are predicted to form between one of the amine groups on Hit 2 and the AA stacked mismatch in the CAG RNA helix. The other amine group is predicted to form hydrogen bonds with the backbone of the neighboring G. In both interactions the NH₂ group on Hit 2 is a hydrogen bond doner, From our studies with structural analogs, the NH₂ groups appear important for reducing expression of polyglutamine protein in CAG repeat expressing screening cell line. NH₂ is predicted to act as hydrogen bond donor for interaction with base sidechain: analogs CAGrb01-A1, CAGrb-04A, CAGrb-09A and CAGrb-013A. And NH₂ is predicted to act as hydrogen bond donor for interaction with backbone: analogs CAGrb-03A, CAGrb-04A, CAGrb-10A, and CAGrb-17A. Binding in the minor groove is predicted to be important for effects of polyQ protein expression: analogs CAGrb01-A1, CAGrb-03A, CAGrb-04A, CAGrb-09A, CAGrb-IDA. CAGrb-13A, CAGrb-17A, CAGrb-18A, CAGrb-21Aand CAGrb-23A.

The structures of the analogs are as follows:

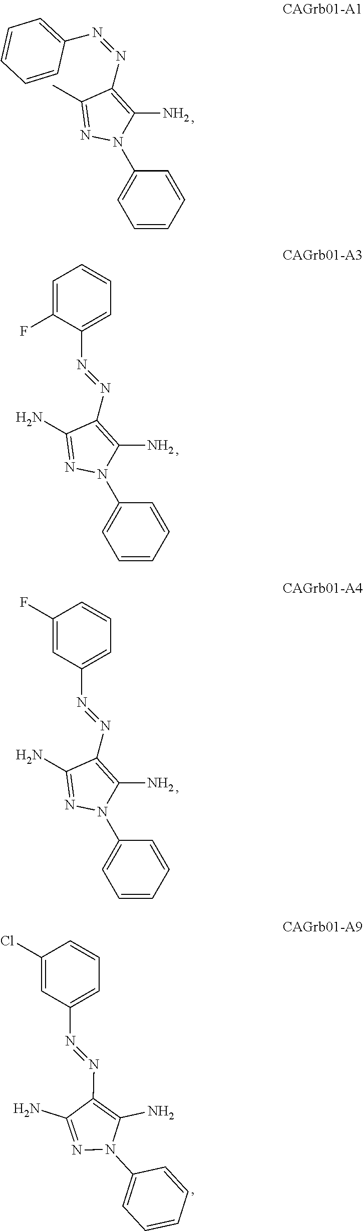 [SMILES: Cc1nn(-c2ccccc2)c(N)c1/N=N\c1ccccc1 ; Nc1nn(-c2ccccc2)c(N)c1/N=N/c1ccccc1F ; Nc1nn(-c2ccccc2)c(N)c1/N=N/c1cccc(F)c1 ; Nc1nn(-c2ccccc2)c(N)c1/N=N/c1cccc(Cl)c1]

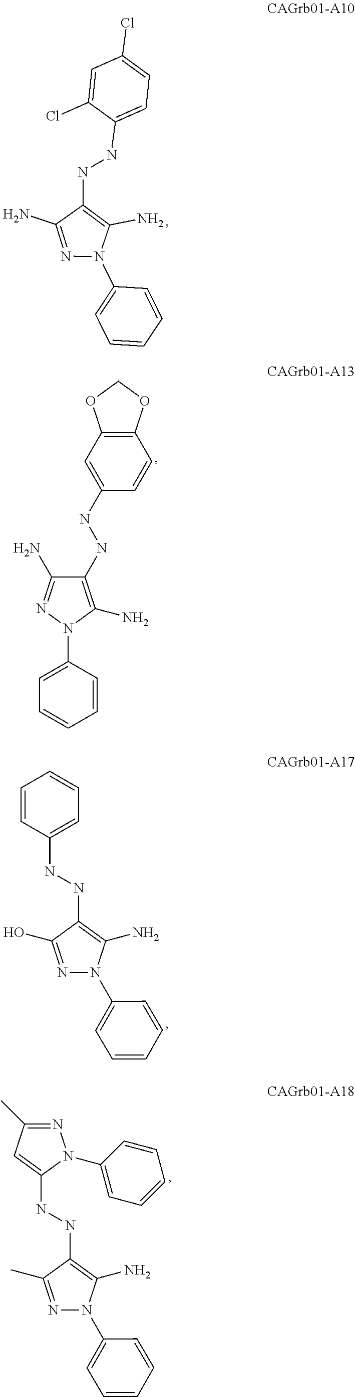

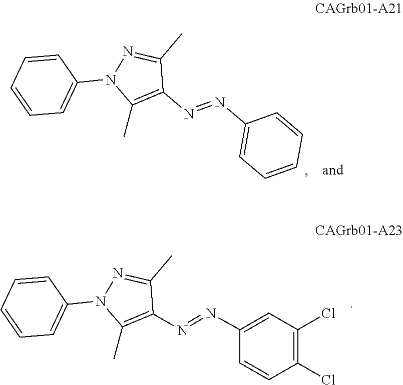 [SMILES: Cc1nn(-c2ccccc2)c(C)c1/N=N/c1ccccc1 ; Cc1nn(-c2ccccc2)c(C)c1/N=N/c1ccc(Cl)c(Cl)c1]

In comparison to the above, the following structural analogs of Hit 2 (CAGrb01) do not show a dose dependent reduction in polyglutamine protein in CAG repeat expressing screening cell lines. The following compounds are shown only to demonstrate the groups that are important for mediating the positive effects seen with the above compounds. Analogs 6, 15, 22, and 24 do not contain NH-2 hydrogen bond donor groups, and analogs 6, 12, 15, and 20 contain a carbonyl group which is predicted to act as a hydrogen bond acceptor in the interaction with CAG RNA helix, Analogs 20 and 22 are not predicted to bind in the minor groove.

The structures of the analogs that do not show a dose dependent reduction in polyglutamine protein in CAG repeat expressing screening cell lines are as follows:

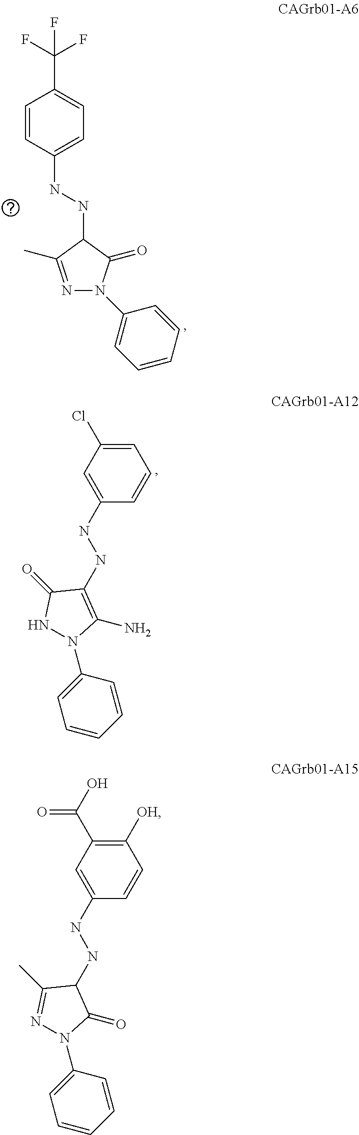

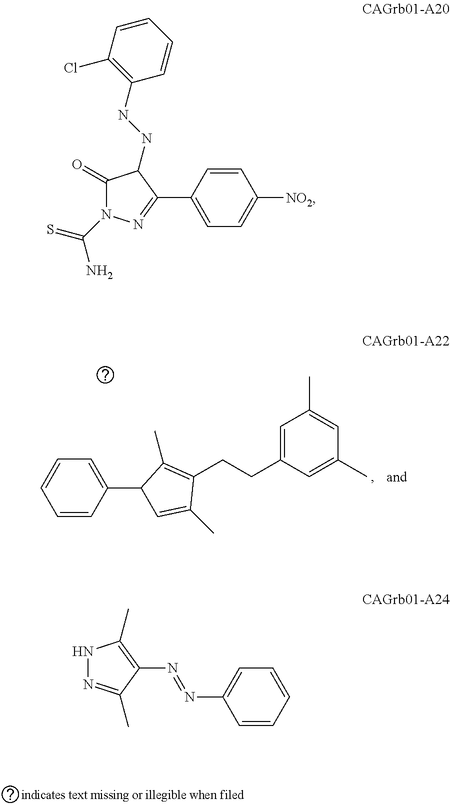

Future studies will be needed to understand whether antisense CUG expansion containing transcripts are expressed in the Atxn1154Q/2Q mouse model and whether some of the off-target effects of Hit 2 on alternative splicing seen are due to upregulation of these antisense transcripts. Overall, this study provides proof of concept that our disease-gene independent, mechanism agnostic small molecule repeat selective screening system is able to identify compounds that regulate GCAG expansion transcript abundance in a repeat dependent manner across multiple GAG expansion diseases. Through this study we have reported the first compounds capable of reducing expression of CAG expansion containing transcripts including our promising lead candidate, Hit 2, which warrants further investigation to improve its solubility and druglike properties. This work also paves the way for further screening studies in a similar repeat selective screening system to identify compounds with the potential for accelerated pace of therapeutic development such as FDA approved compounds. Together this study lays a strong foundation for the potential for repeat selective small molecules as shared therapeutics, either alone or in combination with other therapeutic approaches, across multiple CAG repeat expansion spinocerebellar ataxias, and potentially across the broader class of CAG microsatellite expansion diseases.

### Examples

Methods: Data and material availability: All datasets used in this study are publicly available through the NCBI Gene Expression Omnibus (GEO) under the BioProject accession number PRJNA1049475 using the following GSE numbers: GSE249555 and GSE249556 (Table S1). In house synthesized Hit 2 and the plasmids and protocols used to generate the stably transfected ATG-(CAG)60-Myc-NLuc/ATG-(CAG)0-Myc-FLuc HEK293T cell line are available on request.

Generation and culture of stable CAG repeat expressing HEK293T screening cell line Stable HEK293T cell lines expressing ATG-(CAG)60-Myc-NLuc and ATG-(CAG)0-Myc-FLuc were generated as previously described22. Gene blocks containing the ATG-(CAG)60-Myc-NLuc-Pest and ATG-(CAG)0-Myc-FLuc-Pest cassettes were ordered from Genscript. These were cloned into the constructs we previously used to generate our DM1 screening system22 between the EF1promoter and bGH poly(A) signal using the Miul and Kpnil sites such that the DMPK containing cassette was removed and the PiggyBac transposon terminal repeats, puromycin selection cassette, EF1 promoter and bGH poly(A) signal were retained. The Mlul site was blunted and thus destroyed during the cloning process. These constructs were transfected into HEK293T cells (ATTC) using lipfectamine 3000 (Invitrogen) together with the PiggyBac mPB transposase and selected by puromycin (Gibco). Cells were suspended in a solution of 2% dialyzed HI-FBS (Cytiva), 10 mM HEPES (Sigma) in 1×PBS and single cells were isolated by flow cytometry using a BD FACSAria IIu and colonies were cultured in 96-well plates. Colonies were expanded and subjected to luciferase assay and multiplex qPCR to identify clonal lines that express both ATG-(CAG)60-Myc-NLuc and ATG-(CAG)0-Myc-FLuc. Parental and clonal cell lines were cultured in DMEM (Coming) supplemented with 10% heat-inactivated fetal bovine serum (HI-FBS; Coming) and 1× penicillin/streptomycin (ThermoFisher) in a humidified atmosphere at 37 C at 5% C02; whilst cells expanded from single cells into clones, they were cultured in the same media but with 20% HI-FBS.

Transient transfections of siRNAs and DNA plasmids (CAG)7 siRNA and scrambled control siRNA (Dharmacon) were transfected for a final concentration of 17.5 nM using INTERFERin® (Polyplus) in Opti-MEM (Gibco). For plasmid co-transfections, 1O0 ng of each plasmid was transfected into parental HEK293T cells in a 12 well plate using Lipofectamine 3000 (Invitrogen) in Opti-MEM (Gibco). Diversity Set VI small molecule library screen Approximately 2.5×104 cells per well were plated in a 96 well plate and cultured overnight in 100 μL of DMEM supplemented with 10% HI-FBS and 1×P/S under standard conditions of 37° C. and 5% C02. The next day, medium was removed and replaced with fresh medium (100 μL) containing small molecule at a final concentration of 1 μM or 100 nM or DMSO as a control. All treatments occurred at 0.01% DMSO. The Diversity Set VI library from NC DTP repository was screening in its entirety in clone 37 with subsequent validation screens of initial hits in clones 15 and 37. Each screening plate also contained two wells of each control: DMSO only, 0.1 nM Colchicine, scrambled siRNA and (CAG)7 siRNA. Following 24 hours of treatment, media was removed, and cells were stored at −80° C. The following day, plates were thawed, and each well was incubated with 20 μL of 0.25% Igepal in buffer containing 10 mM Tris-HCl (pH 7.5) and 150 mM NaCl on an orbital shaker for 10 mins at room temperature; 4 μL of “lysate” was then utilized for cDNA generation.

Cell viability analysis and luciferase assay: Cell viability was quantified using the MultiTox-Glo Multiplex Cytotoxicity Assay (Promega) following the manufacturers' instructions. For effects of small molecule treatments on cell viability, data is reported from the live cell component of the assay normalised to DMSO only for each cell line. For assessing cell toxicity of clone 10. 15 and 37, data from the dead cell assay component was normalized to live cells per well, and then to the average for parentals, to account for the difference in growth rate of the cell lines. Poly0-Nluc and zero repeat Fluc were quantified using the Nano-Glo Dual-Luciferase® Reporter Assay System (Promega), following manufacturers' instructions. The Nluc reading was normalised to Fluc per well and then normalised to the average of DMSO only or scrambled siRNA negative control, Cell culture and treatment of CAG SCA patient derived fibroblast lines. Four distinct patient derived fibroblast lines were used in this study (see key resources table): SCA1 (ATXN1: 29/52 repeats), SCA3-1 (ATXN3: 24174 repeats), SCA3-2 (ATXN3: 23/71 repeats) and SCA7 (ATXN7: 7/62 repeats). All control (Table S2; key resources table) and patient derived fibroblast cell lines were cultured in Eagle's Minimum Essential Medium (EMEM; Coming) containing 15% HI-FBS and 1×P/S at 37 C and 5% C02. The cells were expanded and seeded in 12-well tissue culture plates with a density of approximately 3×104 cells/ml. Once the cells reached˜70% confluency, media was removed and replaced with fresh EMEM+FBS+PIS containing Hit 2 or colchicine at the specified concentrations or with a matched percentage of DMSO (vehicle; always≤0.01% DMSO). Prior to adding fresh media containing DMSO or small molecules to the cells, the media/small molecule mix was vortexed for >20s. Following 48 hours treatment, media was removed, and RNA was extracted using Aurum mini kit (BioRad) with on-column DNase I treatment, following the manufacturer's protocol. For analysis of protein expression from Hit 2 treated fibroblast cell lines, after 24 hours treatment, media was replaced with fresh media containing Hit 2 or DMSO at indicated concentration, after an additional 24 hours treatment (48 hours total treatment time) fibroblasts were pelleted and washed in 1×PBS before being lysed. Repeat lengths were assessed using PCR (Key Resources Table) and sanger sequencing. DNA from control and SCA cell lines was extracted using FlexiGene DNA kit (Qiagen) and a PCR across repeat was performed using Phire Hot Start II DNA Polymerase and 5× Reaction Buffer (ThemhoFisher), 04 mM dNTPs (NEB) and primers at a final concentration of 0.4 μM under the following cycling conditions: ATXN1 −98° C. 1 mins-37 cycles of 98° C. 15s, 64° C. 15s, 72° C. 15s −72° C. 1 mins; ATXN3-98° C. 1 mins-35 cycles of 98° C. 15s, 62° C. 15s, 72° C. 15s-72° C. 1 mins; ATXN7-98° C. 3 mins-35 cycles of 98 20s, 65,60° C. 20s, 72VC 1:15s-72° C. 3 mins. The ATXN3 and ATXN7 across the repeat PCRs were supplemented with 1M Betaine (Sigma) and the ATXN7 PCR was also supplemented with 40 μM 7-deaza-dGTP (Roche). PCR products were subjected to gel electrophoresis on a 1% agarose gel and DNA was extracted from PCR bands using EZ-10 Spin Column DNA Gel Extraction Kit (Bio Basic) and sent for DNA sequencing at EtonBio using nested primers.

Culture and treatment of DM1 patient derived myotubes: To assess Hit 2 effects in a CTG repeat expansion disease, control and DM1 myoblasts were seeded in 12-well tissue culture plates in SkGMTM-2 BuiletKit™ growth medium (Lonza) with a density of approximately 1×105cells/mL. Once myoblasts reached˜80% confluency they were differentiated into myotubes for four days in DMEM/F12 50/50 (Corning) supplemented with 2% horse serum. Myotubes were treated with Hit 2 at the specified concentrations or with DMSO (vehicle) in the SkGMTM-2 BulletKit™ growth medium for three days. Cells were harvested after Hit 2 treatment and RNA was extracted using Zymogen's Quick-RNA Miniprep kit with on-column DNase I treatment following manufacturer instructions.

Mouse studies: Mice used in this study were housed and treated in accordance with the NIH Guide for the Care and Use of Laboratory Animals and complied with the Albany Medical College Institutional Animal Care and Use Committee (IACUC) guidelines under approved animal care and use protocol numbers 20-04002 and 23-03002. Atxn1154Q/20 mice were originally obtained from Jackson Laboratories (strain number 005601) and maintained according to established breeding protocols with genotyping performed by PCR, at weaning and retroactively, following existing protocol. Starting at 18 weeks of age, mice were treated with 25 mg/mi Hit 2 dissolved in DMSO or DMSO alone (DMSO<4mli/kg) via intraperitoneal (IP) injection daily for 5 days, followed by two rest days and an additional 5 days of IP injection. All injections were performed between 8 and 9 am in the morning and on the final day of injections (20 weeks of age), after 3 μm, mice were anesthetized using urethane in saline (1.2-1.5 g/kg) followed by a double thoracotomy and perfusion through the ascending aorta with 20 mis 1× PBS. Half the cerebellum, cortex and midbrain, and one liver lobe were removed and stored at −70° C.; the left-brain hemisphere was removed and fixed in 4% PFA for 24 hours, cryopreserved in 30% sucrose for 48 hours and embedded in OCT compound (Fisher); one liver lobe, spleen and kidney were removed and fixed in 4% PFA for 24 hours, stored in 70% ethanol and then processed and embedded by Histowiz Inc. Three days prior to the first injection day and then on days 1, 3, 5, 8, 10 and 12, prior to IP injection, mice were weighed and suspended via their tail for a 10s video to allow for clasping phenotype to be assessed. The clasping phenotype was scored between 0 (no phenotype) and 3 (full clasp) by two independent investigators and the results averaged. The investigators who scored the clasping phenotype were not involved in the treatment regimen and were blinded to the day, genotype and treatment group of the mice. RNA from 6, 12, and 23 week WT and Atxn1154Q/2Q mice harvested as part of a previous study 34 was utilized for validation of dysregulation of specific alternative splicing events in untreated mice across disease onset.

Protein blotting: Media was removed from parental and clonal HEK293T cells and fibroblasts which were then washed with 1×PBS and lysed in radioimmunoprecipitation assay (RIPA) buffer (Thermo Scientific) with 1× complete Protease Inhibitors (Roche) for 15 min on ice for HEK293T cells or 30 min with shaking at 4° C. for fibroblasts. For HEK293T cells, DNA was sheared by passage through a 21-gauge needle. All lysates were centrifuged at 21,000 g for 15 min at 4° C., and the supernatant was collected. The protein lysate concentration was quantified using Pierce BCA Protein Assay Kit (Thermo Scientific), and 10 μg (HEK293T cells) or 25 μg (fibroblasts) of soluble protein lysates was separated on a 4-12% Bis-Tris gel (Bio-Rad) and transferred to a nitrocellulose membrane which was then blocked for 2 hours at room temperature in 5% dry milk in 1×PBS containing 0.05% Tween-20 (Sigma; HEK293T cells) or 5% dry milk in 1×TBS containing 0.1% Tween-20. The membrane was probed with primary antibodies (see Key Resources table) in blocking solution overnight at 4° C., washed three times in 1×PBST (HEK293T) or 1×TBST (fibroblasts) and incubated with species specific secondary antibodies (see Key Resources table; GAPDH was labelled with IRDye 680, all other antibodies were labelled with HRP conjugated secondary antibodies) in blocking solution for 1 hour at room temperature. Following three washes in 1×PBST (HEK293T) or 1×TBST (fibroblasts) at room temperature, ProSignal Pico Spray (Prometheus) was used following manufacturers' instructions and membranes were imaged using a ChemiDoc MP Imaging System (Bio-Rad).

Immunohistochemistry and hematoxylin and eosin staining: Liver, kidney and spleen were sectioned at 4 μm, stained with hematoxylin and eosin (H&E) and imaged at 40× resolution by Histowiz Inc. A board-certified pathologist at Histowiz Inc analysed two liver sections 50 μm apart per mouse. A cryostat was used to section 10 μm sagittal brain sections and H&E was performed on a minimum of three sections 150 μm apart per mouse. H&E staining on sagittal brain sections and imaging at 20× resolution were performed by Albany Medical College Department of Pathology and Laboratory Medicine. For immunostaining with GFAP 10 μm sagittal brain sections were permeabilised in 0.3% Triton-X-100 on ice for 30 mins, heat induced epitope retrieval was performed by placing the slides in a preheated solution of 10 mM sodium citrate (pH 6) at 80 for 40 minutes, cooling to room temperature for 15 minutes with the lid on and then 30 minutes with the lid off. Slides were washed in running water for 10 minutes and endogenous peroxidase activity was blocked by incubating in 3% H₂O₂ in 50% methanol for 10 minutes followed by 10 minutes in running water. Slides were then incubated at room temperature in mouse-on-mouse polymer IHC blocking reagent (abeam) for 45 minutes and incubated overnight at 4° C. with GFAP (1:1000, Antibodies Inc) in a 1:10 dilution of background sniper (Biocare Medical) in 1×PBS. Slides were washed 3×5 min in 1×PBS, incubated with mouse-on-mouse polymer IHC detector reagent (abeam) for 15 mins at room temperature, washed 3×5 min in 1×PBS and incubated with ImmPACT® DAB EqV Substrate Kit (Vector laboratories) for 1 minute 30 seconds, washed in running water for 10 minutes, incubated in Hematoxylin QS Counterstain (Vector laboratories) for 5 seconds and washed in running water for 10 minutes before mounting (abcam). GFAP stained sections were imaged at 20× resolution by Albany Medical College Department of Pathology and Laboratory Medicine.

Reverse transcription: RNA concentrations were measured using nanodrop and 500 ng total RNA was reverse transcribed using SuperScript IV reverse transcriptase (Invitrogen) with random hexamers (IDT) for patient derived fibroblast lines, cerebellum, liver, and qPCRs in FIG. 11, or RT-primer (see key resources table) for multiplex qPCR for CAG repeat containing cell lines. Reverse transcriptase reactions were performed with 200 ng of input RNA for cDNA production for Control and DM1 myotubes.

Quantitative PCR (RT-qPCR) analysis: Multiplex qPCR was carried out using Hot Start Taq 2× Master Mix (NEB) with Multiplex qPCR Fw and Multiplex qPCR Rv primers and Multiplex qPCR Probe 1 and Multiplex qPCR Probe 2 fluorescent probes (all IDT; see Key Resources table). Hepatotoxicity was assessed using an array of 24 predesigned PrimePCR assays from the BioRad mouse Hepatotoxicity SAB target list with the qPCR performed using Ssoadvanced Master Mix (BioRad) according to manufacture instructions: one target, Pdyn, was not detected in multiple mice across all treatment groups and so could not be analysed. For all other qPCRs cDNA was subjected to qPCR for 39 cycles with PowerUp SYBR Master Mix (Applied Biosystems) or Ssoadvanced Master Mix (BioRad) according to manufacturer's instructions. All qPCRs were performed in a CFX384 or CFX96 Real-Time System (Bio-Rad). All qPCR reactions were performed in technical triplicates or quadruplicates using 1-3 uL cDNA depending on expression of target gene (i.e. 3 uL input for GABRA3 qPCR, 1 uL input for GAPDH). Ct values were obtained via CFX maestro software (Bio-Rad) and RT-qPCR data were analysed using the 2—Ct methods 6. The levels of r(CAG)60 from small molecule treatments were normalized to r(CAG)0 and presented as relative mRNA levels by comparing to DMSO control treatments. GAPDH was used as the housekeeping gene. To confirm specificity of primers, qPCR was performed on RT reactions in which the RT enzyme was replaced with H2O (RT-) under the same conditions. All primers are listed in the key resources table.

RT-PCR splicing analysis: PCR for selected splicing events was performed using the Taq 2× master mix (NEB) with 2 uL cDNA under the following conditions: 95° C. 30s-32 cycles of 95° C. 30s, primer specific Tm 30s, 68° C. 30s-68° C. 5 min. Primer sequences are included in the key resources table. Annealing temperatures and product sizes (inclusion, exclusion) are as follows: MBNL1 exon 5 (Ta 58° C.; 308 bp, 250 bp); MBNL2 exon 5 (Ta 58° C.; 266 bp, 211 bp); SYNE1 exon 137 (Ta 58° C.; 146 bp, 83 bp), Cacna2d2 exon 23 (Ta 51° C.; 107 bp, 86 bp). PCR products were resolved through capillary electrophoresis in a 5300 Fragment Analyzer system using the DNF-905 kit for 1-500 bp fragments (Agilent Technologies), following the manufacturer's protocol. The relative fluorescence values (RFU) for the inclusion and exclusion bands, obtained from the ProSize 4.00 software (Agilent technologies), were used to calculate percent spliced in (PSI) for each exon of interest using the following formula: (inclusion band RFU)/(Inclusion band RFU+Exclusion band RFU)*100. For alternative splicing analysis for Atxn1154Q/2Q and WT mice PCR products were resolved in technical triplicates on the fragment analyser and the average PSI is reported for each sample.

RNA sequencing: RNA for CAG containing reporter cell lines was extracted using Aurum total RNA mini kit (BioRad) with on-column DNase treatment, following the manufacturer's protocol. RNA for wild-type and KI mice was extracted using TRIzol (Ambion, Life Technologies), following the manufacturers protocol and a DNA digestion was performed using the TURBO DNA-free Kit (Invitrogen). The Qubit RNA high sensitivity assay was used to obtain RNA concentrations (Thermo Fisher Scientific). RNA quality was checked via capillary electrophoresis on a 5300 fragment analyzer (Agilent Bioanalyzer) using the RNA Analysis DNF471 RNA kit (Agilent). The NEBNext Ultra II Directional RNA Library Prep Kit (Illumina) with NEBNext rRNA Depletion Kit (New England Biolabs) was used to prepare RNA-seq libraries, with a total of 500 ng input RNA from each sample. The manufacturer's protocols were followed, with the following exceptions: 40× adaptor dilutions were used, and 13 cycles of library amplification were performed. The resulting libraries were pooled in equimolar amounts, quantified using the NEBNext Library Quant Kit for Illumina (New England Biolabs), quality checked via capillary electrophoresis on the 5300 fragment analyzer (Agilent Bioanalzyer) using the DNF-474 HS NGS kit (Agilent).

Libraries with sufficient quantity for RNA Seq of mouse cerebellum were loaded into P2 flow cell 1000/2000 by Illumina and were sequenced using paired-end, 100 base pair sequencing on the Illumina NextSeq 2000 sequencer at the University at Albany RNA Institute. Libraries with sufficient quantity for RNA Seq of parental, clone 15 and clone 37 cell lines sequenced using paired-end, 75 base pair sequencing (parental and clone 37), or 100 base pair sequencing (clone 15) on the Illumina NextSeq 500 the University at Albany Center for Functional Genomics. All datasets had total read depths greater than 40 million reads (Table S1).

FASTQ file quality was assessed using FastOC (version 0.11.9). Both paired-end files for samples 409, 422, 425, and 426 were downsampled using Seqtk (version 1.4) to either −65 or 70 million reads per file to more closely match the read depth of the other samples in the dataset. FASTO files were then aligned to the GRCm39/mm39 mouse reference genome or GRCh38/hg38 genome using STAR with the-quantModeGeneCounts option added to count the reads per gene (version 2.7.10a)57. Differential gene expression was performed in RStudio (2022.12.0; R 4.2.2) using DESeq2 (version 3.16)58 and genes that passed a threshold of padj<0.05 and log 2FC>|1.01 were considered significantly differentially expressed. Volcano plots and heatmaps were created in RStudio (R version 4.2.2) using the packages Enhanced Volcano and p heatmap respectively. Differential gene expression events were considered off-target if they were significantly different between WT-DMSO and Atxn1154/2Q-Hit 2 (log 2FC>Ji,01 and Padj<0.05) but showed no difference between WT-DMSO and Atxn1154Q/2Q-DMSO (log 2FC<10.51) mice. Alternative splicing analysis was performed using rMATS (version 4.1.2)59 and events were considered significantly misspliced if the false-discovery rate (FDR)<0.1 and ΔPSI>|0.1|All ΔPSI values are converted from a ratio to a percentage with the threshold adjusted accordingly: ΔPSI>I10%1. Dysregulated skipped exon events were considered off-target if they were significantly differentially regulated between WT-DMSO and Atxn1154Q/2Q-Hit 2 or Atxn1154Q/2Q-DMSO and Atxn1154Q/2Q-Hit 2 mice (ΔPSI>|0.1| and FDR<0.1) but showed no difference between WT-DMSO and Atxn1154Q/2Q-DMSO (ΔPSI<(0.1| and P>0.05). Exon numbers are referred to by previously published exon numbers for the same coordinates or based on counting the first exon of the relevant transcript of a gene as exon 1. Coordinates for reported exons are as follows: Trpc3 exon 9 chr3:36688523-1336688607), Ankslb exon 5 (chr10:90750557-90750629), Atp13a4 exon 24 (chr16:29234600-1429234697), Wnk1 exon 4b (chr6:119905083-119905110), Zbtb20 exon 5 (chr16:43392096-1543392154), and Cacna2d2 exon 23 (chr”:107396377-107396398). Gene ontology enrichment analysis was performed using Metascape (version v3,5.20230101)60.

Synthesis of r(CAG)10: The r(CAG)10 oligonucleotide was synthesized using an Oligo 800 DNA/RNA Synthesizer at a scale of 1.Opmol via solid phase synthesis. Phosphoramidites obtained from ChemGenes were dissolved in dry acetonitrile to obtain a concentration of 0.07M and were immobilized using guanosine phosphoramidite containing control-pore glass (CPG-1000 Å) beads. Detritylation was carried out using 3% Trichloroacetic Acid in Dichloromethane followed by the coupling of the dissolved phosphoramidite using 5-ethylthiotetrazole in acetonitrile as the activation reagent. Unreacted phosphoramidites were capped using acetic anhydride in pyridine/tetrahydrofuran (1:1:8) and 16% n-methylimidazole in tetrahydrofuran.

Oxidation was done using 0.02 M iodine in water/pyridineltetrahydrofuran (2:20:78). This was repeated for the full sequence. The RNA oligonucleotides were cleaved from the CPG beads using 8001L 1:1 v/v ammonium hydroxide (28% NH3 in H2O) and methylamine (40% w/w aqueous) at 65° C. for 45 minutes. After this time, the solution was transferred to new 1.5 mL tubes and concentrated using a Speed-Vac concentrator. After concentration and drying, the RNA was dissolved in 100 μL DMSO and deprotected using triethylamine trihydrofluoride for 2.5 hours at 65° C. Ethanol precipitation was performed to the specifications of Cold Spring Harbors with the pellet being dried to completion and redissolved in DI H2O. The resulting solution was desalted using 200 μL C18 cartridges from Waters Corp. Columns were equilibrated using 1 mL Dry Acetonitrile, 1 mL DI H2O, and 1 mL 2M Triethylamine Acetic Acid (TEAAc). 1 mL of RNA sample was loaded onto the column via gravity filtration. Subsequent washes to remove salt contamination were performed using 1 mL DI H2O three times using gently air flow at a rate of 1 drop/sec. followed by unloading of the purified RNA from the column using 1 mL 1:1 Acetonitrile:DI H2O using high air pressure, Fractions were combined and dried to completion. 1 mL of DI H2O was added to resuspend the RNA pellet.Purity of the RNA was verified using PAGE Gel Electrophoresis, r(CAG)10 oligonucleotides were diluted in DI H2O for use.

Fluorescent Indicator Displacement (FID) assay: FID assays enable sensitive and quantitative detection of the interaction between a small molecule and a receptor, in this case CAG repeat RNA, based on the displacement of an indicator whose fluorescent properties differ when it is bound versus not bound to the receptor 6l. Here we conducted FID assays to examine the interactions between Hit 2 and (CAG)10 RNA in the presence of increasing concentrations of Hit 2. The synthesised (CAG)10 RNA in dH2O was diluted in an assay solution consisting of 1× sodium cacodylate buffer (contains 10 mM sodium cacodylate, 25 mM NaCl, and 1 mM EDTA). The solution was then annealed by heating it to 95° C. for 5 minutes, followed by an incubation at room temperature for one hour. For optimisation of the FID assay conditions, a dose range of both (CAG)10 RNA and SYBR Safe between 100 nM and 6 μM were tested before selecting the optimal concentrations used here to ensure reliable and reproducible readouts. After incubation, the (CAG)10 RNA was transferred to half well black assay plate and 100 nM SYBR Safe was added along with increasing concentrations of Hit 2, from 0 μM to 10 μM, such that the final concentration of (CAG)10 RNA was 1 μM. The mixture was further incubated at room temperature for an additional 45 minutes. The fluorescence (F) was recorded using an excitation wavelength of 500 nm and an emission wavelength of 530 nm. The change in fluorescence was recorded on a microplate reader (Synergy™ H1 multi-mode microplate reader). The percentage of SYBR Safe displacement was calculated using the following equation:

$$\% Displacement = 100 - ( F * 100 ) F 0$$

where F=F(Hit2+RNA+SYBR safe)−F (buffer+SYBR safe)−F (RNA+Hit2) F0=F(RNA+SYBR safe)−F (buffer+SYBR safe) The final data was plotted and a curve fitted using SIGMA plot 15.0 software. The p-value for the dissociation constant (Kd) was calculated using a t-test for the parameter estimate. This test evaluates the significance of the Kd value by comparing the estimated parameter to zero, providing a measure of statistical significance. UV melting assay UV melting assays were performed to determine the thermal stability of the target RNA in the presence of increasing concentrations of Hit 2. (CAG)10 RNA oligonucleotides (IDT) were prepared by dissolving them in 1× sodium cacodylate buffer (containing 10 mM of Sodium cacodylate, 25 mM of NaCl. and 1 mM of EDTA), followed by annealing through heating at 95° C. for 5 minutes and incubated for 1 h at room temperature. Melting curves were recorded by monitoring the absorbance at 260 nm as the temperature was increased from 20° C. to 95° C. at a rate of 1° C. per minute using a Cary 3500 Multicell UV-Vis Spectrophotometer. Each experiment was performed using 2 μM RNA, with Hit2 concentrations varying from 0 μM to 10 NM. Due to changes in Hit 2 concentration affecting baseline absorbance (FIG. 271), each measurement was normalized by subtracting the corresponding blank values consisting of Hit2 (from 0 μM to 10 μM) and buffer only. After normalized the values the data was plotted using SIGMA Plot 15.0 software, and the melting temperature (Tm) for each sample was calculated using MeltWin 3.5 software.

Computational modelling of Hit 2 binding to CAG and CUG RNA: To perform molecular docking studies for Hit 2 bound to CAG and CUG RNA, suitable RNA structures were identified from the PBD database. The RNA used for CAG repeats is a mixed sequence structure with a CAG helical region and a UUCG loop (PBD ID: 7D12)62. For CUG RNA, a CUG duplex was used (PBD ID: 1ZEV)63. To investigate intercalation, an intercalation site was created by constructing a r(CAG)3 RNA duplex and then generating the intercalation site using the rebuilding feature of web 3DNA 2.064. These structures were energy minimized and molecular docking was performed using Molecular Operating.

Environment (MOE) (chemoompcom/Products): First, structure preparation, involved correcting protonation states and topologies of both the RNA and Hit 2. Next molecular docking was performed using the triangle matcher and london dG method for placement of Hit 2 and scoring of the complexes respectively, to find the top 50 binding modes. These were then refined using induced fit method to allow for some local flexibility and scored using GBVI/WSA dG method to obtain the top 10 binding modes. The interaction between Hit 2:RNA complex structures were analyzed in MOE and visualized in PyMOL (The PyMOL Molecular Graphics System. Version 2.3.5, Schrodinger, LLC).

Preparation of Hit: Hit 2 (IUPAC name: 4-[(4-methylphenyl)diazenyl]-1-phenylpyrazole-3,5-diamine; CAS number: 5456-92-8) was synthesized as previously described33. Briefly p-toluidine (1g, 1 eq) was suspended in 2.5 mL of 6N HCl (1.25 mL of water and 1.25 mL of concentrated HCl) and cooled in ice/water bath. Sodium nitrite (1.3 eq in 5 mL of water) was then added portion wise while stirring. This mixture was then added to a cold solution of sodium acetate (1.1 eq), malononitrile (1 eq) in 10 mL of 50% aqueous ethanol. The thick precipitate was then stirred vigorously for 2 hrs and filtered to afford a bright yellow solid, 853 mg of which was carried onto the next step. Phenylhydrazine (1 eq) was then added in ethanol and heated to 60° C. overnight. This was then cooled and filtered to give a light brown solid, ˜900 mg. FIG. 308 confirms the synthesized 4[(4-methylphenyl)diazenyl]-1-phenylpyrazole-3,5-diamine (Hit 2) matched that of FIG. 30A obtained from the NCI DTP (NSC: 21883; CAS: 5456-92.8): each compound was analyzed by the Agilent 65308 LC/MS OTOF mass spectrometer with dual ESI source. The fragmentor and skimmer voltage were set to 175 V and 65 V, respectively, while the voltage amplitude and capillary voltage were set to 750 V and 4000 V, respectively. The mobile phase was a mixture of water with 0.1% formic acid and acetonitrile (ACN) also containing 0.1% formic acid at gradient conditions:

| | Water | ACN | |
|---|---|---|---|
| | w/0.1% | w/0.1% | |
| Time | Formic | Formic | Flow |
| (min) | Acid (%) | Acid (%) | (mL/min) |
| 0 | 95 | 5 | 1 |
| 4 | 5 | 95 | 1 |
| 4.05 | 5 | 95 | 1.5 |
| 5 | 5 | 95 | 1.5 |
| 5.05 | 95 | 5 | 1 |
| 6 | 95 | 5 | 1 |

¹H NMR and ¹³C NMR were performed using a Bruker 500 Ultrashield™ spectrometer. The ¹H NMR data are depicted as chemical shift multiplicity: s (singlet), d (doublet), t (triplet), number of protons, and coupling constant. ¹H NMR (500 MHz, DMSO-d6) 0 ppm: 2.33 (s, 3H), 7.22-7.23 (d, J=8.60 Hz, 2H), 7.27-7.30 (t, J=7.59 and 7.63 Hz, 1H), 7.47-7.50 (t, J=8.12 and 7.76 Hz, 2H), 7.57-7.58 (d. J=815 Hz, 2H). 7,66-7.67 (d. J=8.57 Hz, 2H) (FIG. 30C). ¹³C NMR (500 MHz, DMSO-d6) δ ppm: 21.979. 114.887, 120.590, 120.590, 122.015, 122.015. 123.322, 123.322, 126.915, 128,915, 129.144, 130.393, 130.567. 137.176. 139.242, 151.836 (FIG. 30D). (m/z): [M+H]+for molecular formula [C16H16N6+H+]: calculated 293.197; found 293.197.

Quantification of in vivo Hit 2 concentration: Development and optimization of a method for in vivo Hit 2 quantification was based on previous studies using a HPLC approach for small molecule quantification. To generate calibration curves for quantification of in vivo Hit 2 concentration, cortex and midbrain from wild-type mice was homogenized in 100% methanol, vortexed for 2 minutes, centrifuged at 17115g for 20 mins at 4° C. and the supernatant was collected and then filtered through a 0.22 μm filter and Hit 2 was spiked into the lysate at known concentrations. The filtered supernatant or Hit 2 in 100% methanol at the same concentrations was subjected to HPLC analysis on a Shimadzu model LC-20AB (Shimadzu, USA) with a UV detector (SPD-20SV) using an Agilent C-18 Eclipse column (4.6×150 mm, 5 μm particle size). The flow rate was 1 mL/min, with the UV lamp set at 254 nm, and the loop volume at 20 μL. For the mobile phase, we employed two solvents: Solvent A, consisting of distilled water and 0.1% TFA (Trifluoroacetic acid), and Solvent B, composed of Acetonitrile and 0.1% TFA. We implemented a gradient method wherein the mobile phase began as 97% solvent A and 3% solvent B for 2 minutes. Subsequently, the percentage of solvent B was increased gradually to 45%, 75%, and 85% for 2 minutes each, followed by an increase to 90% for 4 minutes. Then, solvent B was adjusted to 3%, and at 15 minutes the run was stopped. A peak for Hit 2 was consistently observed at a retention time of 5.4-5.6 mins. The area under the peak was quantified using the software LabSolution (Shimadzu, USA); to do this, horizontal lines were drawn under the peak to separate peak area from background. Mouse brain samples were prepared as above for quantification of in vivo Hit 2 in wild-type DMSO injected mice, Atxn1^{154Q/2Q}-DMSO and Atxn1^{154Q/2Q}-Hit 2 mice except, due to the low quantity of tissue available for this analysis (average weight of cortex and midbrain used per mouse: 48.38 mg), the filtering step was removed to prevent loss of sample in the filter. This resulted in the baseline shifting from ˜0 mV to −250 mV (FIG. 5). 15 μL of samples were injected in duplicate for generation of the standard curve and in triplicate for in vivo quantification of Hit 2. To validate and assess accuracy of the standard curve, three samples were injected five times over two days.

To confirm that the peak at 5,4-5.6 min corresponded to Hit 2 in Atxn1^{154Q/2Q}-Hit 2 mice, the three peaks from HPLC of cortex and midbrain lysate from mouse #421 were collected and injected into a mass spectrometer (Agilent Technologies, 6530, Accurate-Mass Q-TOF LC/MS, USA). Based on the calculation of m/z=293.1618 [292.346+H+]from analysis of the spectra using AgtMass-HunterAcquisition software (Agilent, USA), we can confirm the identification of Hit-2 in the HPLC peak at 5.4-5.6 min retention time.

Data analysis For qPCR and alternative splicing validation analyses, statistical analysis was performed using GraphPad Prism 9. Grubb's test with an alpha of 0.001 was used to identify and remove outliers for Atxn1^{154Q} qPCR. Data are represented as mean t standard error of the mean (SEM), mean t standard deviation (SD) or median with range as appropriate which is listed in the figure legends. Statistical analyses were performed using two-tailed Student's unpaired t-test, one-way ANOVA with Tukey's multiple comparisons test, or two-way ANOVA with Tukey's multiple comparisons test as appropriate and are listed in figure legends. A critical value for significance of p<0.05 was used throughout the study.

TABLE S1
RNA Sequencing datasets

| PRINA1049475 | ID | Sex | Genotype | Treatment | RIN score | Total read-depth | Downsampled total read-depth |
|---|---|---|---|---|---|---|---|
| GSE249556 | 413 | M | Wt | DMSO | 8 | 147668268 | NA |
| | 425 | F | Wt | DMSO | 10 | 226178630 | 130000000 |
| | 426 | F | Wt | DMSO | 10 | 217174296 | 130000000 |
| | 431 | M | Wt | DMSO | 9.2 | 122989072 | NA |
| | 409 | M | Het | DMSO | 6.4 | 212785844 | 130000000 |
| | 411 | M | Het | DMSO | 7 | 88591702 | NA |
| | 420 | M | Het | DMSO | 9.8 | 87317374 | NA |
| | 429 | M | Het | DMSO | 8.3 | 129594098 | NA |
| | 410 | M | Het | Hit 2 | 8.5 | 86298122 | |
| | 421 | M | Het | Hit 2 | 7.5 | 108777144 | NA |
| | 422 | M | Het | Hit 2 | 9.8 | 239793282 | 140000000 |
| | 430 | M | Het | Hit 2 | 9.5 | 11221174 | NA |
| GSE249555 | P1_Parental | NA | Parental | NA | NA | 39997046 | NA |
| | P2_Parental | NA | Parental | NA | NA | 46576068 | NA |
| | P3_Parental | NA | Parental | NA | NA | 49212254 | NA |
| | C1_Clone37 | NA | Clone 37 | NA | NA | 40939306 | NA |
| | C2_Clone37 | NA | Clone 37 | NA | NA | 40565810 | NA |
| | C3_Clone37 | NA | Clone 37 | NA | NA | 44406142 | NA |
| | C1_Clone15 | NA | Clone 15 | NA | 10 | 139405850 | NA |
| | C2_Clone15 | NA | Clone 15 | NA | 10 | 92028910 | NA |
| | C3_Clone15 | NA | Clone 15 | NA | 10 | 68553816 | NA |

TABLE S2
CAG repeat lengths for ATXN1, ATXN3 and ATXN7 in control

| Cell line ID | | | | CAG repeat length | | |
|---|---|---|---|---|---|---|
| This study | Original source | Age | Sex | ATXN1 | ATXN3 | ATXN7 |
| Ctrl1 | DM04 | 45 | M | 28 | 17 | 10 |
| Ctrl2 | GM08400 | 37 | F | 28 | 17 | 10 |
| Ctrl3 | GM23249 | 44 | M | 29 | 16 | 7 |

What we claim is:

1. A method for treating a subject having a CAG repeat expansion disorder, the method comprising: administering to the subject a therapeutically effective amount of a compound that selectively reduces the level of transcripts containing expanded CAG repeats, wherein the compound is a small molecule and is not an antisense oligonucleotide, short hairpin RNA, or microRNA.

2. The method of claim 1, wherein the compound is: 4-((4-methylphenyl)diazenyl)-1-phenylpyrazole-3,5-diamine.

3. The method of claim 1, wherein the compound is colchicine or a derivative thereof.

4. The method of claim 1, wherein the compound is at least one selected from the group consisting of:

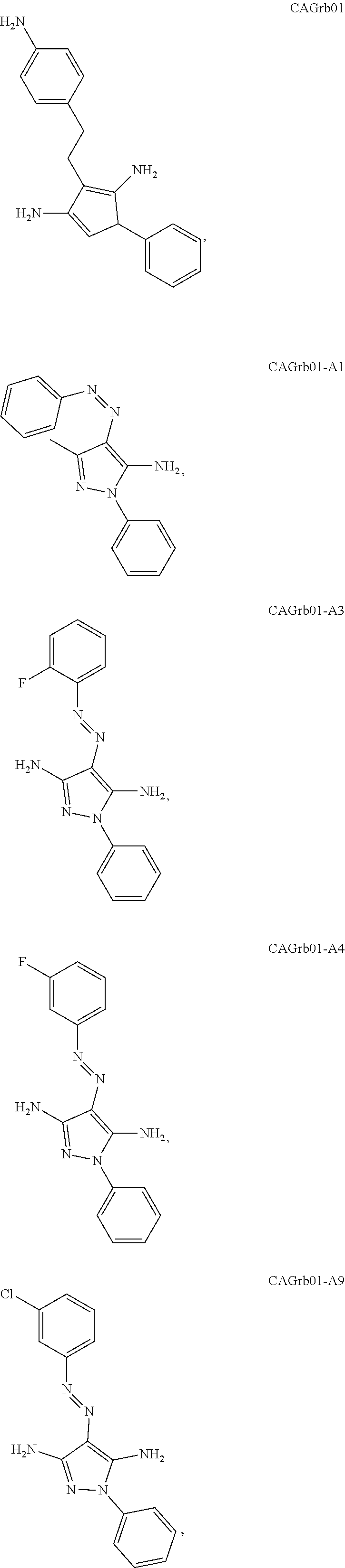 [SMILES: NC1=CC(c2ccccc2)C(N)=C1CCc1ccc(N)cc1 ; Cc1nn(-c2ccccc2)c(N)c1/N=N\c1ccccc1 ; Nc1nn(-c2ccccc2)c(N)c1/N=N/c1ccccc1F ; Nc1nn(-c2ccccc2)c(N)c1/N=N/c1cccc(F)c1 ; Nc1nn(-c2ccccc2)c(N)c1/N=N/c1cccc(Cl)c1]

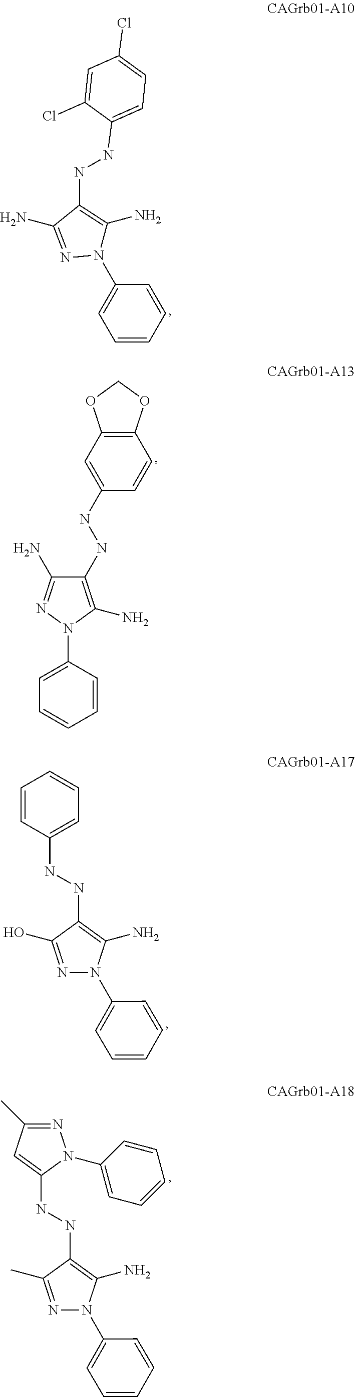

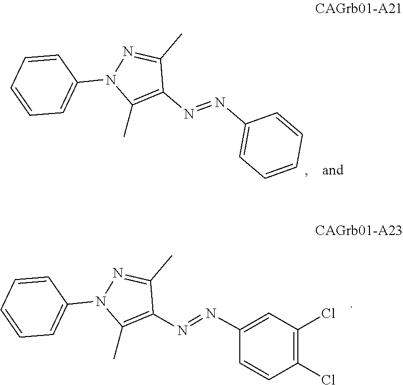 [SMILES: Cc1nn(-c2ccccc2)c(C)c1/N=N/c1ccccc1 ; Cc1nn(-c2ccccc2)c(C)c1/N=N/c1ccc(Cl)c(Cl)c1]

5. The method of claim 1, wherein the CAG repeat expansion disorder is selected from the group consisting of: spinocerebellar ataxia type 1 (SCA1), type 2 (SCA2), type 3 (SCA3), type 6 (SCA6), type 7 (SCA7), type 8 (SCA8), type 17 (SCA17), Huntington's disease, dentatorubral-pallidoluysian atrophy (DRPLA), and spinal and bulbar muscular atrophy (SBMA).

6. The method of claim 1, further comprising the step of assessing alternative splicing correction in the subject, wherein the compound rescues dysregulated splicing caused by CAG repeat expansion.

7. A method for identifying a therapeutic compound for treating a CAG repeat expansion disorder, the method comprising:
(a) providing a cell expressing a transcript comprising an expanded CAG repeat;
(b) contacting the cell with a test compound;
(c) measuring the level of the transcript comprising the expanded CAG repeat in the cell; and
(d) identifying the test compound as a candidate therapeutic if the level of the transcript is selectively reduced compared to a control, wherein the test compound is not an antisense oligonucleotide.

8. The method of claim 7, wherein the compound is

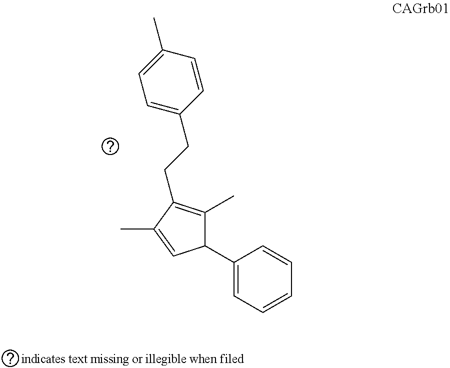 [SMILES: CC1=CC(c2ccccc2)C(C)=C1CCc1ccc(C)cc1]

9. The method of claim 7, wherein the compound is selected from the group consisting of:

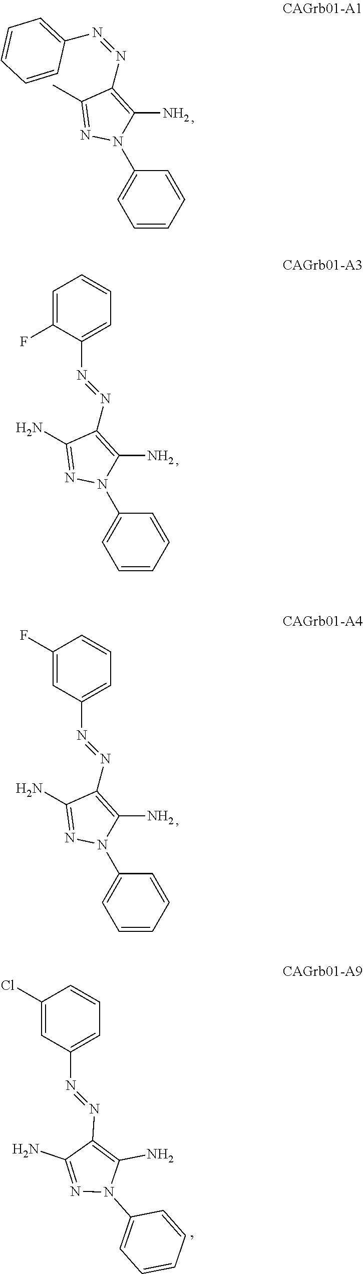 [SMILES: Cc1nn(-c2ccccc2)c(N)c1/N=N\c1ccccc1 ; Nc1nn(-c2ccccc2)c(N)c1/N=N/c1ccccc1F ; Nc1nn(-c2ccccc2)c(N)c1/N=N/c1cccc(F)c1 ; Nc1nn(-c2ccccc2)c(N)c1/N=N/c1cccc(Cl)c1]

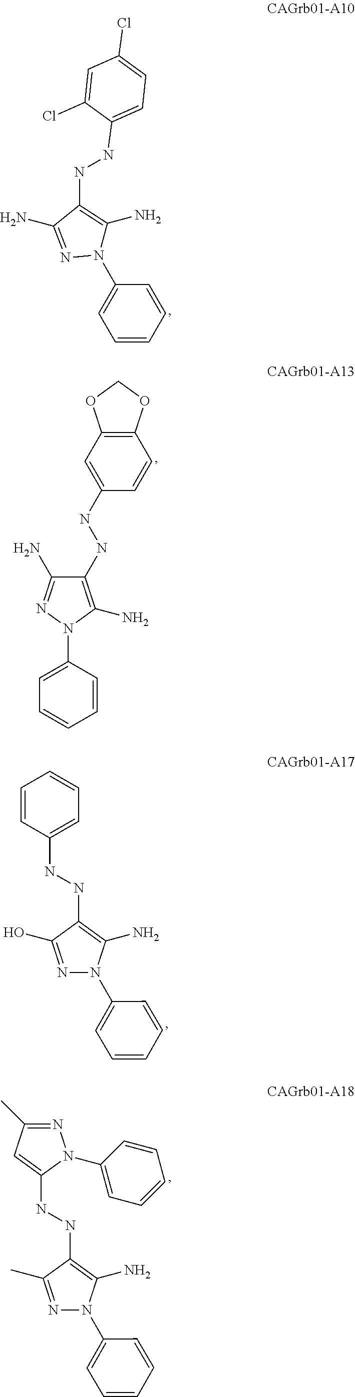

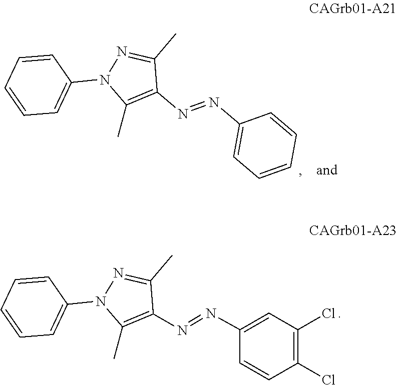 [SMILES: Cc1nn(-c2ccccc2)c(C)c1/N=N/c1ccccc1 ; Cc1nn(-c2ccccc2)c(C)c1/N=N/c1ccc(Cl)c(Cl)c1]

10. A pharmaceutical composition comprising, a small molecule that binds to CAG repeat RNA and selectively reduces expanded CAG transcript levels; and a pharmaceutically acceptable carrier, wherein the small molecule is identified using the method of claim 7.

11. A method for identifying compounds that selectively reduce the expression of CAG repeat-containing RNA and associated polyglutamine proteins, the method comprising:
culturing a mammalian cell line co-expressing: a first reporter construct comprising at least 60 CAG trinucleotide repeats operably linked to a reporter gene encoding a nanoluciferase-polyglutamine fusion protein; and a second reporter construct lacking CAG repeats and encoding a firefly luciferase control protein;
contacting the cell line with a candidate compound;
measuring expression levels of: (i) the CAG repeat-containing RNA using RT-qPCR, and (ii) the polyglutamine-nanoluciferase fusion protein using a luciferase assay;
identifying the compound as a selective modulator of CAG expansion RNA and polyglutamine protein expression if it reduces the expression of the CAG repeat-containing RNA by at least about 15%, and the expression of the polyglutamine fusion protein by at least about 40%, compared to untreated or vehicle-treated controls.

12. The method of claim 11, wherein the candidate compound is selected from the National Cancer Institute Diversity Set VI.

13. The method of claim 11, wherein the compound is a pyrazole-based compound.

14. The method of claim 11, wherein the cell line is a HEK293T clone expressing integrated constructs via a PiggyBac transposon system.

15. The method of claim 11, wherein the compound is 4-[(4-methylphenyl)diazenyl]-1-phenylpyrazole-3,5-diamine.

16. The method of claim 11, wherein the compound is selected from the group consisting of:

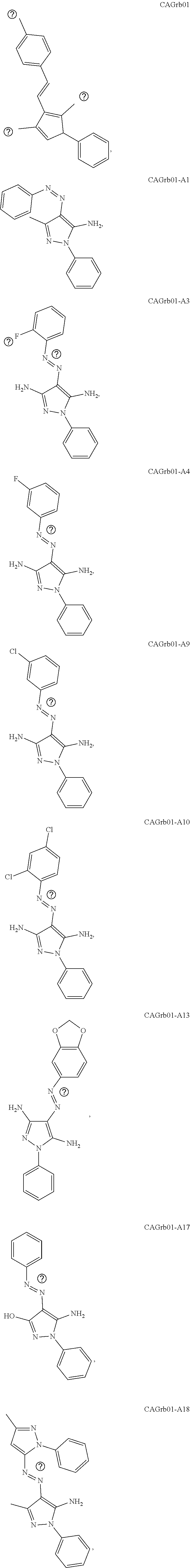

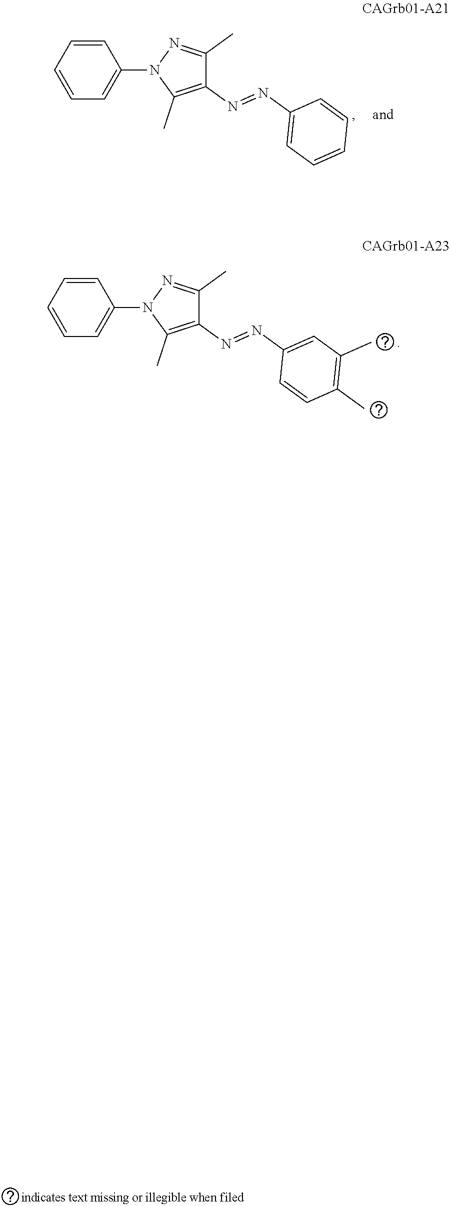 [SMILES: Cc1nn(-c2ccccc2)c(C)c1/N=N/c1ccccc1 ; Cc1ccc(/N=N/c2c(C)nn(-c3ccccc3)c2C)cc1C]

17. A cell-based screening system for identifying compounds that modulate expression of CAG repeat-containing RNA, comprising:
a eukaryotic host cell line co-expressing: a first reporter construct comprising an open reading frame encoding a polyglutamine anoluciferase fusion protein, wherein the open reading frame includes at least 60 CAG repeats, and a second reporter construct comprising an open reading frame encoding firefly luciferase, wherein the open reading frame includes 0 CAG repeats;
wherein each reporter construct comprises a unique probe-binding sequence located downstream of the CAG repeat region; wherein the expression levels of the first and second reporter constructs are independently quantifiable via both:
(i) a dual luciferase assay, and
(ii) a multiplex RT-qPCR assay using fluorescent probes directed to the unique probe-binding sequences; and
wherein the cell line is adapted for high-throughput screening of candidate therapeutic compounds targeting CAG repeat expansion disorders.

18. A cell-based screening method for identifying CAG RNA-modulating compounds, comprising:
(a) culturing the cell line of claim 17;
(b) administering test compounds at concentrations S10 μM:
(c) quantifying (CAG)₆₀ and (CAG)₀ RNA levels via multiplex qPCR; and
(d) selecting compounds showing 220% reduction in (CAG)₆₀/(CAG)₀ RNA ratio with Z′ score≥0.51.

19. An engineered cell line for identifying compounds that modulate CAG repeat-containing RNA expression, comprising:
(a) a first integrated nucleic acid construct encoding (CAG)₆₀-Myc-NLuc under an EF1 promoter;
(b) a second integrated nucleic acid construct encoding (CAG)₀-Myc-FLuc under the EF1 promoter; and
(c) a puromycin resistance gene (Puro-R) and bidirectional stop cassettes (6xStop) flanking each construct to prevent readthrough transcription.

20. The cell line of claim 19, wherein the constructs are integrated into HEK293T cells to generate a plurality of clones that exhibit:
(a) (CAG)₆₀ RNA expression and (CAG)₀ RNA, as measured by a multiplex qPCR comprising FAM- and HEX-labeled probes;
(b) Z* scores≥0.5 in RNA reduction assays; and
(c) differential expression of SCA-associated genes consistent with CAG expansion disease models.
